(19) United States
(12) Patent Application Publication
(10) Pub. No.: US 2022/0340643 A1
(43) Pub. Date: Oct. 27, 2022

(54) AAV-COMPATIBLE LAMININ-LINKER POLYMERIZATION PROTEINS

(71) Applicant: Rutgers, The State University of New Jersey, New Brunswick, NJ (US)

(72) Inventors: Peter D. Yurchenco, New Brunswick, NJ (US); Karen K. McKee, New Brunswick, NJ (US)

(21) Appl. No.: 17/642,399

(22) PCT Filed: Sep. 11, 2020
(86) PCT No.: PCT/US2020/050530

§ 371 Date: Mar. 11, 2022

Related U.S. Application Data
(60) Provisional application No. 62/900,236, filed on Sep. 13, 2019.

(51) Int. Cl. C07K 14/78; C12N 15/86

(52) U.S. Cl. CPC C07K 14/78; C12N 15/86; C12N 2750/14143; A61K 48/00

(57) ABSTRACT

The present invention relates to recombinant laminin adeno-associated viral vector (AAV) constructs and related methods for restoring laminin expression in deficient mammals, or in mammals with basement membrane instability.

### CROSS-REFERENCE TO RELATED PATENT APPLICATIONS

This application is claiming priority to the International Application No. PCT/US2019/031369, filed May 8, 2019, which claims benefit of U.S. Provisional Patent Application No. 62/900,236, filed on Sep. 13, 2019, which is incorporated by reference in its entirety.

### STATEMENT OF GOVERNMENT SUPPORT

This invention was made with government support under grant number R01-DK36425 awarded by the National Institutes of Health. The government has certain rights in this invention.

### SEQUENCE LISTING

The instant application contains a Sequence Listing which has been filed electronically in ASCII format and is hereby incorporated by reference in its entirety. Said ASCII copy, created on Sep. 11, 2020 is named 10491_006542-W01_ST25.txt and is 229 KB (234,545 bytes) in size.

### FIELD OF THE INVENTION

The present invention relates to recombinant laminin adeno-associated viral vector (AAV) constructs and related methods for restoring laminin expression in deficient mammals, or in mammals with basement membrane instability.

### BACKGROUND

Laminins are essential components of basement membranes (BMs) and their assembly. These large glycoproteins are heterotrimers consisting of α-, β- and γ subunits joined in a long coiled-coil. The fundamental role of laminins is to create a primary scaffold that (1) attaches the extracellular matrix to the cell surface and cytoskeleton and (2) that serves as a platform to which other extracellular matrix components, such as the nidogens, collagens and perlecan/agrin heparin sulfate proteoglycans, become stably attached.

Many different types of diseases involve basement membranes and laminins. Metastasizing solid tumors must pass through basement membranes to reach the vascular system, and various microbes and viruses enter the cells through direct interaction with laminins. At least nine of the laminins are essential for life based on genetic evidence in mice. Mutations in the laminin N-terminal (LN) polymerization domain of several laminins are causative of muscle, nerve, and kidney diseases. See, Scheele et al., 2007 J Mol Med 85(8):825-36.

Laminin-211 (a heterotrimer consisting of α2, β1 and γ1 subunits, abbreviated as Lm211) is the major laminin of the basement membranes of skeletal muscle and peripheral nerve Schwann cell (SC) and is found also in brain capillaries. See, Aurnailley et al., (2005) Matrix Biol 24(5):326-32.

During embryogenesis, the laminin α2 chain is expressed along developing muscles from embryonic day 11 of development. LN domain mutations within the LAMA2 gene coding for the laminin α2 chain can result in a complete or near-complete loss of laminin α2 protein subunit expression to cause laminin α2-deficient muscular dystrophy (LAMA2-MD). LAMA2-MD is an autosomal recessive disease that typically presents as a non-ambulatory congenital muscular dystrophy (CMD), also known as congenital muscular dystrophy type 1A (MDC1A), a particularly severe non-ambulatory congenital dystrophy that begins at birth or infancy and is often accompanied by involvement of peripheral nerve and brain.

A recent study of 249 LAMA2 MD patients in United Kingdom revealed that LAMA2 mutations were the most common (37.4%) followed by dystroglycanopathies and Ullrich-CMD. See, Sframeli, et al., (2017) Neuromuscul Disor 27(9): 793-803. There are also a small number of missense and inframe deletion mutations, mostly mapping to the laminin α2 short-arm polymerization domain (LN), that cause a milder ambulatory dystrophy. See, Allamand, et al., (1997) Hum Mol Genet 6(5):747-52; Gavassini, et al., (2011) Muscle Nerve 44(5):703-9; Bonnemann, et al., (2014) Neuromuscul Disord 24(4):289-311; Chan, et al., (2014) Neuromuscul Disord 24(8):677-83. The pathology in both consists of muscle degeneration, regeneration, chronic inflammation and fibrosis accompanied by white matter brain anomalies and reduced peripheral nerve conduction. See, Jimenez-Mallebrera, et al., (20025) Cell Mol Life Sci 62(7-8):809-23. Patients with null-expression mutations never ambulate, can have peripheral nerve conduction defects, seizures and moderate mental retardation, and often die of muscle wasting and respiratory failure at a young age. Patients with defective α2-laminin present later in life with a less severe ambulatory form of dystrophy, typically limb-girdle type, and also exhibit peripheral and central nervous system defects. See, Bonnemann, et al., (2014) Neuromuscul Disord 24(4):289-311. Treatment generally focuses on managing the individual signs and symptoms of the condition. There is currently no cure for either.

Another neuromuscular disease, Pierson syndrome, is associated with a deficiency of the laminin β2 chain, which is prominently expressed in the glomerular basement membrane at the neuromuscular junctions, as well as in the intraocular muscles, lens and retina. The laminin β2 chain deficiency is caused by missense and in-frame deletion mutations of the LAMB2 gene. Pierson syndrome is an autosomal recessive disease, a very rare condition that mainly affects the kidneys and eyes. Most affected children have early-onset, chronic renal failure, neurodevelopmental problems, distinct eye abnormalities that may include blindness, hypotonia, psychomotor delay, hemiparesis and abnormal movements. See, Schéele et al., (2007) J Mol Med 85:825-836. Affected infants may not survive past the first weeks or months of life. Those that survive past infancy typically have neurological disabilities and developmental delays. Most require a renal transplant for end-stage kidney disease within the first decade of life. The long-term outlook is poor.

There is an ongoing need for better treatments, especially for gene therapy to restore laminin polymerization expression and basement membrane assembly in patients, and in particular for treating diseases involving laminin α2 and laminin β2 deficiencies.

### SUMMARY OF INVENTION

In certain embodiments, the present invention relates to a recombinant adeno-associated vector (rAAV) comprising a nucleic acid sequence comprising a transgene encoding alphaLNNdDeltaG2short (αLNNdΔG2′). In certain embodiments, the αLNNdΔG2′ comprises SEQ ID NO: 1. In certain embodiments, the rAAV further comprises a CMV promoter comprising SEQ ID NO: 12. In certain embodiments, the rAAV is AAV8 or AAV-DJ. In certain embodiments, the rAAV further comprises inverted terminal repeats (ITRs). In certain embodiments, the ITRs are a 5′ ITR comprising SEQ ID NO: 11 and a 3′ ITR comprising SEQ ID NO: 16.

In certain embodiments, the present invention relates to a composition comprising any of the recombinant AAV's described herein. In certain embodiments, the composition further comprises a pharmaceutical carrier.

In certain embodiments, the present invention relates to a kit comprising a container housing comprising the composition described herein. In certain embodiments, the container is a syringe.

In certain embodiments, the present invention relates to a method of restoring laminin polymerization expression and basement membrane assembly in a subject, comprising administering to the subject an effective amount of any of the recombinant AAV vectors described herein.

In certain embodiments, the present invention relates to a method of treating laminin α-2 deficiency in a subject in need thereof, comprising administering to the subject an effective amount of any of the recombinant AAV vectors described herein.

In certain embodiments, the present invention relates to a method of alleviating in a subject at least one of the symptoms associated with laminin deficiencies selected from the group consisting of laminin-deficient muscular dystrophies and laminin α2-deficient muscular dystrophy, wherein the method comprises administering to the subject an effective amount of any of the recombinant AAV vectors described herein.

In certain embodiments, the present invention relates to a method of alleviating in a subject at least one of the symptoms associated with laminin α2-deficiencies selected from the group consisting of muscle degeneration, regeneration, chronic inflammation, fibrosis, white matter brain anomalies, reduced peripheral nerve conduction, seizures, moderate mental retardation, and respiratory failure, wherein the method comprises administering to the subject an effective amount of any of the recombinant AAV vectors described herein.

In certain aspects, embodiments of the invention relate to a method for treating laminin α2-deficient muscular dystrophy in a subject characterized by the defect or haploinsufficiency of an LAMA2 gene. The method may include administering to the subject an effective amount of a recombinant adeno-associated virus carrying a nucleic acid sequence (i.e., a transgene) encoding an alphaLNNdDeltaG2short (αLNNdΔG2′), under the control of a promoter sequence which expresses the αLNNdΔG2′ product in the desired cells. In certain embodiments, the promoter sequence provides for expression of the αLNNdΔG2′ product in basement membranes. In certain embodiments, expression of the transgene gene provides to the cells the product necessary to restore or maintain desired laminin polymerization expression and basement membrane assembly in the subject. In still another embodiment, the invention provides a composition for treatment of laminin α2-deficient muscular dystrophy. Such compositions may be formulated with a carrier and additional components suitable for injection.

Other aspects and advantages of the present invention are described further in the following detailed description of the preferred embodiments thereof.

### BRIEF DESCRIPTION OF THE DRAWINGS

FIG. 1 illustrates the neuromuscular laminin interactions with core basement membrane (BM) components. Relevant laminin and other protein domains are labeled. Dashed and dotted lines indicate domain binding interactions. Abbreviations: laminin (Lm); laminin 111 (Lm111); laminin 411 (Lm411); sulfated glycolipids (SGL); α-dystroglycan (αDG); nidogen (Nd); Lmα2 short-arm polymerization domain (LN).

FIG. 2 illustrates a model of Lm211 and Lm411 mediated BM assembly in muscle and peripheral nerve. Abbreviations: laminin 211 (Lm211); laminin 411 (Lm411); sulfated glycolipids (SGL); α-dystroglycan (αDG); nidogen (Nd); Lmα2 short-arm polymerization domain (LN); N-terminal domain of agrin that binds to laminin coiled-coils (agrin-NtA); laminin G-like domain (LG).

FIGS. 3A-E are illustrations, EM images and SDS-PAGE images showing linker protein repair of laminin function. FIG. 3A shows the domain structure and functional activities of αLNNd and mag. Regions derived from laminin-α1 are in green; regions derived from nidogen-1 are in orange. Mag is a miniaturized version of agrin with N-terminal regions (blue) and C-terminal parts (red). FIG. 3B shows rotary shadowed EM images of αLNNd and mag, and complexes with laminins. FIG. 3C shows that in the ambulatory form of LAMA2 MD and its dy2J/dy2J mouse model, a truncated version of Lm-211(“dy2J-Lm-211”) is expressed. αLNNd binds to the nidogen-binding site and creates an artificial short arm with a functional LN domain. Co-expression of αLNNd and mag provide the necessary domains for polymerization and αDG anchorage. FIG. 3D shows shortened versions of polymerization linker proteins lacking G2 domain±2 EGF-like repeats, i.e., αLNNd, αLNNdΔG2, and αLNNdΔG2. FIG. 3E shows linker-laminin complex formation of αLNNdΔG2 with Lmα1ΔLN-L4b.

FIG. 4 shows shortened versions of αLNNd polymerization linker proteins lacking G2 domain±2 EGF-like repeats, i.e., αLNNd (alphaLNNd where alpha refers to laminin-alpha1, LN refers to the LN domain, and Nd refers to nidogen), αLNNdΔG2 (alphaLNNdDeltaG2), and αLNNdΔG2′ (alphaLNNdDeltaG2short).

FIGS. 5A-E are SDS-PAGE, immunofluorescent images, and a graph showing AAV expression of αLNNdΔG2′ and mag bound to Lm411 and assembly of αLNNdΔG′-Lm4l 1 on Schwann cells. FIGS. 5A and 5B show, respectively, αLNNdΔG2′-AAV and mag5myc-AAV infection of 293 cells expressing Lm411. Complex with Lm411 is shown by immunoprecipitation of N-terminal FLAG-tagged Lm411 from medium followed by cutting the membrane with immunoblotting of the upper segment for Lmα4 and the lower segment for αLNNdΔG2′ in FIG. 5A or mag and αLNNdΔG2′ in FIG. 5B. FIGS. 5C and 5D show a substantial increase of Lm411 assembly resulted from AAV-generated αLNNdΔG2′. FIG. 5E shows the detection in sarcolemma of antibody stained αLNNdΔG2′ (red) and laminins (green) from the i.m. injection of AAV-αLNNdΔG2′ into a 1 week old dy3K/dy3K, mag Tg mouse.

FIG. 6 is a map of the pAAV-MCS expression vector.

FIG. 7 is a map of the pAAV-DJ Vector.

FIG. 8 is a map of the pHelper vector.

FIG. 9 is a comparison of the mouse and human amino acid sequences for the αLNNdΔG2′ protein using a protein BLAST alignment. Query=the human αLNNdΔG2′ amino acid sequence. Subject—the mouse αLNNdΔG2′ amino acid sequence.

FIG. 10 provides the nucleotide and amino acid sequences of the open reading frame of the mouse αLNNdΔG2′ (short-noG2) as inserted in an AAV. The signal peptide is encoded by nucleotides 1 to 51 (Color: Green). Lma1 LN is encoded by nucleotides 52 to 804 (Color: Blue). LEa1 is encoded by nucleotides 805 to 975 (Color: Magenta). LEa2 is encoded by nucleotides 976 to 1185 (Color: Green). LEa3 is encoded by nucleotides 1186 to 1356 (Color: Red). Lea4 is encoded by nucleotides 1357 to 1503 (Color: Cyan). Lma1 LF segment is encoded by nucleotides 1504 to 1536 (Color: Blue). Nd egf-4 is encoded by nucleotides 1537 to 1668 (Color: Red). Nd egf-5 is encoded by nucleotides 1669 to 1809 (Color: Cyan). NdTY is encoded by nucleotides 1810 to 2091 (Color: Magenta). Nd G3 is encoded by nucleotides 2092 to 2835 (Color: Green). Nd egf-6 is encoded by nucleotides 2836 to 3006 (Color: Red).

FIG. 11 provides the nucleotide and amino acid sequences of the open reading frame of the human αLNNdΔG2′ (short-noG2) as inserted in an AAV. The signal peptide is encoded by nucleotides 1 to 51 (Color: Green). Lma1 LN is encoded by nucleotides 52 to 804 (Color: Blue). LEa1 is encoded by nucleotides 805 to 975 (Color: Magenta). LEa2 is encoded by nucleotides 976 to 1185 (Color: Green). LEa3 is encoded by nucleotides 1186 to 1356 (Color: Red). LEa 4 is encoded by nucleotides 1357 to 1503 (Color: Cyan). LF fragment is encoded by nucleotides 1504 to 1536 (Color: Blue). Nd egf-4 is encoded by nucleotides 1537 to 1668 (Color: Red). Nd egf-5 is encoded by nucleotides 1669 to 1809 (Color: Cyan). NdTY is encoded by nucleotides 1810 to 2091 (Color: Magenta). Nd G3 is encoded by nucleotides 2092 to 2835 (Color: Green). Nd egf-6 is encoded by nucleotides 2836 to 3006 (Color: Red).

FIG. 12 provides the nucleotide sequence of the open reading frame of the mouse αLNNdΔG2′ (short-noG2) as inserted in an AAV.

FIG. 13 provides the amino acid sequence of the mouse αLNNdΔG2′ (short-noG2).

FIG. 14 provides the nucleotide sequence of the open reading frame of the human αLNNdΔG2′ (short-noG2) as inserted in an AAV.

FIG. 15 provides the amino acid sequence of the human αLNNdΔG2′ (short-noG2).

FIG. 16 provides a graph showing the results of linker protein mediation of laminin assembly on cultured myotubes. The linker proteins αLNNd (indicated as [1]), αLNNdALEa3,4 (indicated as [2]), αLNNdΔG2′ (indicated as [3]) and αLNNdA2EGF′ (indicated as [4]) all showed substantial and significant increased laminin on the myotube surfaces compared to the non-polymerizing laminin control.

FIGS. 17A-C provide graphs showing the results of nidogen competition for selected linker proteins and competition between three linker proteins and nidogen-1 (Nd) on C2C12 myotubes. FIG. 17A shows the results of competition between linker αLNNd (ABCDEFGHIJKLMNOP) and nidogen-1. FIG. 17B shows the results of competition between linker αLNNdΔ2EGF′ (ABCDHIJNOP) and nidogen-1. FIG. 17C shows the results of competition between linker αLNNdG2′ (ABCDEFLMNOP) and nidogen-1.

FIGS. 18A-C are immunostained images and a bar graph showing the results of non-polymerizing laminin incubated with a reduced-size linker protein (code: ABCHIJNOP) and then added to the medium of cultured Schwann cells. FIG. 18A is an immunostained image that shows the non-polymerizing laminin assembly on Schwann cell surfaces. FIG. 18B is an immunostained image that shows the increased accumulation of laminin with the gain of function of polymerizing protein. FIG. 18C is a graph providing a quantitative comparison of FIGS. 18A and 18B. None represents no laminin. Lmα1ΔLN-L4b refers to laminin 111 lacking the α1 short arm polymerization domain. Nd refers to nidogen-1. Col4 and C4 refer to Type IV collagen ABCHIJNOP refers to αLNNdΔ2EGF′ minus LEa2, EGF3. Rel. Lmγ1 immunofluor./cell is the relative Lmγ1 immunofluorescence divided by the number of counted cells. “av.” refers to average.

### DETAILED DESCRIPTION

The heterotrimeric laminins are a defining component of all basement membranes and self-assemble into a cell-associated network. In mammals, all laminins are heterotrimers composed of one of five α chains, one of three β chains and one of three γ chains. Despite a total of at least 45 potential αβγ chain combinations, only 15 different laminin isoforms were reported as of 2010. Based on in vitro studies, there are at least 16 allowed laminin isoforms (Table 1 below).

TABLE 1
Mammalian laminins.^{1 2}

| Name | Abbreviated Name | Chain composition |
|---|---|---|
| Laminin-111 | Lm111 | α1β1γ1 |
| Laminin-121 | Lm121 | α1β2γ1 |
| Laminin-211 | Lm211 | α2β1γ1 |
| Laminin-213 | Lm213 | α2β1γ3 |
| Laminin-221 | Lm221 | α2β2γ1 |
| Laminin-311³ | Lm311 | α3β1γ1 |
| Laminin-312⁴ | Lm312 | α3β1γ2 |
| Laminin-321 | Lm321 | α3β2γ1 |
| Laminin-332 | Lm332 | α3β3γ2 |
| Laminin-411 | Lm411 | α4β1γ1 |
| Laminin-421 | Lm421 | α4β1γ1 |
| Laminin-422⁵ | Lm422 | α4β2γ2 |
| Laminin-423 | Lm423 | α4β2γ3 |
| Laminin-511 | Lm511 | α5β1γ1 |
| Laminin-521 | Lm521 | α5β2γ1 |
| Laminin-523 | Lm523 | α5β2γ3 |

¹Table based on P.R. Macdonald et al., 2010, J. Struct. Biol. 170: 398-405.
²Note:
Little is known of the subunit partners or tissue distribution of the laminin β4 subunit.
³The laminin α3 subunit can exist as shorter (A) and longer (B) splice variants sharing the same coiled-coil and LG domains. The B variant additionally possesses a short arm with an LN polymerization domain. The α3B variant is thought to assemble with the same β- and γ- subunits as α3A.
⁴While it is uncertain if Lm212 exists in vivo, its assembly has been detected in vitro.
⁵While it is uncertain if Lm422 exists in vivo, its assembly has been detected in vitro.

Laminins are essential central organizers of basement membranes, a likely consequence of the unique ability of laminins to bind to cells, to self, and to other basement membrane components. Basement membranes, which are required for the emergence of tissues and differentiated cells, are important in embryo development, tissue homeostasis and human disease.

The three short arms of the cross-shaped laminin molecule form the network nodes, with a strict requirement for one α, one β and one γ arm. The homologous short arms are composed of a distal laminin N-terminal (LN) domain that is followed by tandem repeats of laminin-type epidermal growth factor-like (LE) domains, interspersed with globular domains of unknown structure. The LN domains are essential for laminin polymerization and BM assembly. Laminin polymerization is also important for myelination. Laminins containing the α3A, α4, and β2 subunits do not have a full complement of LN domains and therefore cannot polymerize (reviewed in Hohenester and Yurchenco. 2012. Cell Adh. Migr. 2013. 7(1):56-63).

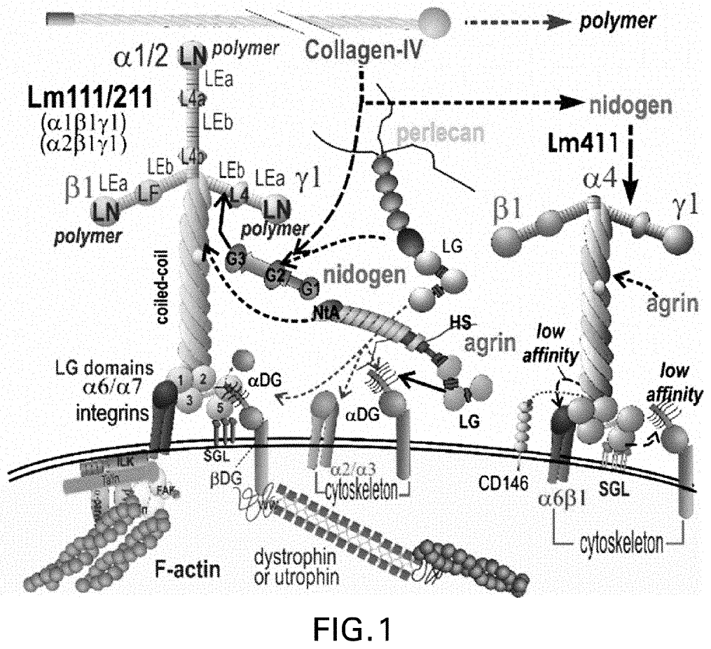

The long arm of the cross (75-80 nm length) is an α-helical coiled coil formed from all three chains, whereas the three short arms (35-50 nm) are composed of one chain each. At the distal end of the long arm, the α chain adds five laminin G-like (LG) domains that contain the major cell-adhesive sites of laminin. This globular domain at the end of the long arm binds to cellular receptors, including integrins, α-dystroglycan, heparan sulfates and sulfated glycolipids. Collateral anchorage of the laminin network is provided by the proteoglycans perlecan and agrin. A second network is then formed by type IV collagen, which interacts with the laminin network through the heparan sulfate chains of perlecan and agrin and additional linkage by nidogen. See generally, Hohenester et al. (2013) Cell Ahd Migr. 7(1):56-63. This maturation of basement membranes becomes essential at later stages of embryo development. In FIG. 1, Lm111, a prototypical laminin (Lm) expressed in embryogenesis, binds to cell surface sulfated glycolipids (SGL), integrins, α-dystroglycan (αDG), nidogen (Nd), agrin, and polymerizes via its LN domains. Collagen-IV and perlecan bind to nidogen. Integrin and αDG attach through adaptor proteins to the cytoskeleton. Lm411, a Lm isoform that does not polymerize, exhibits very weak integrin and αDG binding.

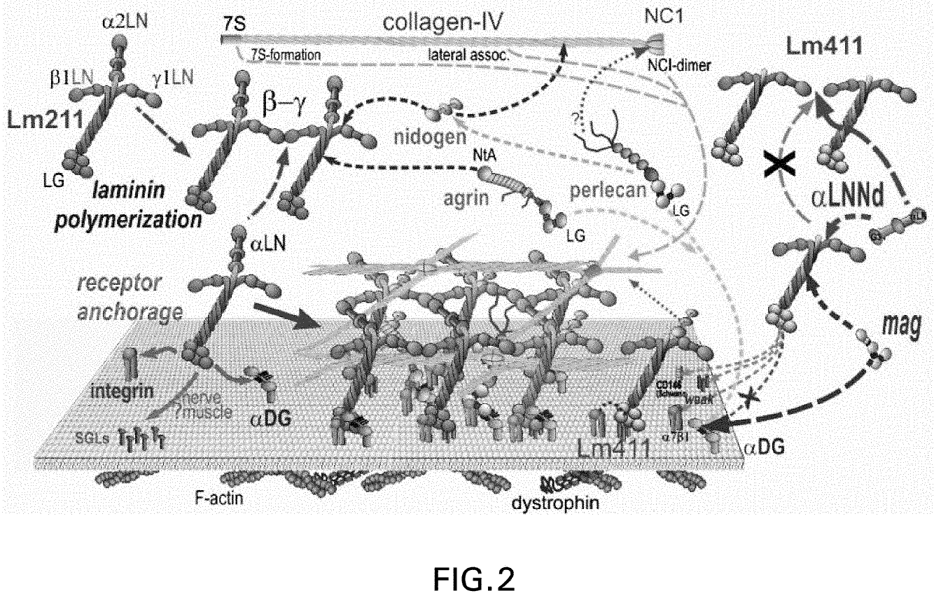

Lm211 and Lm411 mediate BM assembly in muscle and peripheral nerve. The laminin forms the initial nascent scaffolding by binding to sulfated glycolipids (SGL) such as sulfatides, binding to integrin α7β1 and α-dystroglycan (αDG), and polymerizing via LN interactions, illustrated in FIG. 2. Nidogen (mostly nidogen-1) binds to laminin and to collagen-IV, acting as a bridge, with the collagen polymerizing to form a second network. All components become directly or indirectly tethered to cell receptors through laminin but can separately interact with other integrins. Lm411 is a non-polymerizing laminin that co-assembles with Lm211 in nerves. αLNNd binds to Lm411 and imparts polymerization activity. Miniagrin (mag, mA) binds to Lm411 and imparts αDG binding. (See McKee et al. 2017. J. Clin. Invest. 127: 1075-1089 and Reinhard et al. 2017, Sci. Transl. Med. 28:9 (396), pii: eaal4649. doi: 10.1126/scitranslmed.aa14649).

Schwann cell (SC) BMs share the overall architectural organization with muscle BMs; however, they differ in several respects: (i) β1-integrins are the major mediators of myelination whereas in muscle αDG is the paramount receptor; (ii) several SC integrins are available to interact with BM (but only α7β1 in muscle), allowing integrin ligation of other BM components; (iii) Lmα4, absent in myofibers, is a normal SC subunit that contributes to myelination; (iv) SCs express sulfatides and CD146 that may enable α4-laminin adhesion; and (v) Dy2J amyelination is most evident in the sciatic nerve and roots, suggesting a special importance of laminin polymerization. Alpha 2-laminin is also found in capillaries forming the blood-brain barrier. Loss of the laminin subunit makes the barrier leaky to water, likely explaining the brain white matter changes detected by MRI in nearly all LAMA2-MD patients.

Laminin α 2-deficient muscular dystrophy (LAMA2-MD) is an autosomal recessive disease caused by mutations within the LAMA2 gene that typically presents as a non-ambulatory congenital muscular dystrophy (CMD). The dystrophy is often accompanied by involvement of peripheral nerve and brain. The great majority of LAMA2 mutations result in a complete or near-complete loss of protein subunit expression, in particular Lm211, to cause a particularly severe non-ambulatory congenital dystrophy. There are also a small number of missense and in-frame deletion mutations, mostly mapping to the Lm α short-arm polymerization domain (LN), that cause a milder ambulatory dystrophy. In LAMA2-MD, there is increased transcription and protein accumulation of Lm411, with minor increases in Lm511. Lm411 is unusual in that it binds weakly to muscle αDG and integrins and lacks the ability to polymerize. Lm411 is inadequate for BM assembly such that high Lm411 concentrations are required for cell surface accumulation relative to other laminins, which explains its limited ability to rescue LAMA2 mutations. These compositional changes underlie the structural attenuations of the BM seen in the absence of laminin-α2. See review, Yurchenco et al. 2017, Matrix Biology, pii: S0945-053X(17)30333-5. doi: 10.1016/j.matbio.2017.11.009.

Several mouse models for the laminin α2 chain deficiency are available, and they also display muscular dystrophy and peripheral and central nervous system myelination defects. BMs are disrupted, and the expression of LM α2-chain receptors and some BM associated proteins are altered in the LM α2-chain deficient muscles, and both structural and signaling defects may be detrimental for normal muscle function. Furthermore, critical roles for laminin α2 chain inducing Schwann cell proliferation and oligodendrocyte spreading, as well as myelination in the peripheral nervous system and central nervous system, respectively, have been demonstrated. See, Schéele et al., (2007) J Mol Med 85:825-836. Laminin α2 is greatly reduced in dyW (dy^{W}/dy^{W}) mice while completely absent in dy3K (dy^{3K}/dy^{3K}) Lama2-knockout mice. These two models represent the majority of LAMA2-MD patients that either express very low or no laminin α2 subunit at all. The dy3K mice, the most severely affected of the mice, are extremely weak, small, and very short-lived. A third model is the dy2J (dy^{2J}/dy^{2J} genotype) mouse in which laminin α2 is slightly decreased while laminin α4 is modestly increased. Lm211 in dy2J mice is unable to polymerize because of the loss of the LN-domain. Dy2J mice are characterized by progressive weakness and paralysis beginning at about 3½ weeks of age with the hindlimbs affected first and later the axial and forelimb musculature, Schwann cells fail to sort and ensheathe axons resulting in amyelination. These mice, however, can survive many months.

There are challenges for development of a treatment for LAMA2-MD. A direct approach of restoring laminin expression by germ-line transgenesis of Lama1 (Lma1) has been effective in its ability to restore normal function in mice; however, the 9.3 kb DNA construct is too large for available delivery systems. Drug therapies show improvements, but importantly do not correct the underlying structural defect. EHS-derived Lm111, delivered to inflamed muscle parentally, has been found beneficial in dyW mice, but this approach has not been shown to be effective with recombinant laminin, which would be needed for treatment. While exon-skipping to correct out-of-frame mutations has been used to treat dystrophin-deficiency, it is problematic for laminin-deficiency in that exon borders do not match protein domain borders and skipping of nearly all LAMA2 exons will likely result in cysteine mispairing and domain misfolding. AAV-delivered CRISPR/Cas9 has been used to repair splice defects, which are found in approximately 20% of LAMA2-MD subjects. Transgenic minagrin (mag) expression was shown to partially ameliorate the muscle pathophysiology of mouse models of laminin-α2-deficient muscular dystrophy, even when expressed after birth. Similar benefits were observed when a mag gene was introduced into perinatal dyW (dyW/dyW) mice by AAV. See, Qiao, et al., Proc Natl Acad Sci USA (2005) 102(34):11999-2004. Micro-dystrophin AAV delivery to treat Duchenne muscular dystrophy in humans has been demonstrated. See, Mendell, Neurosci Lett (2012). The present invention provides a repair of basement membranes with potential to improve all LAMA2-MDs.

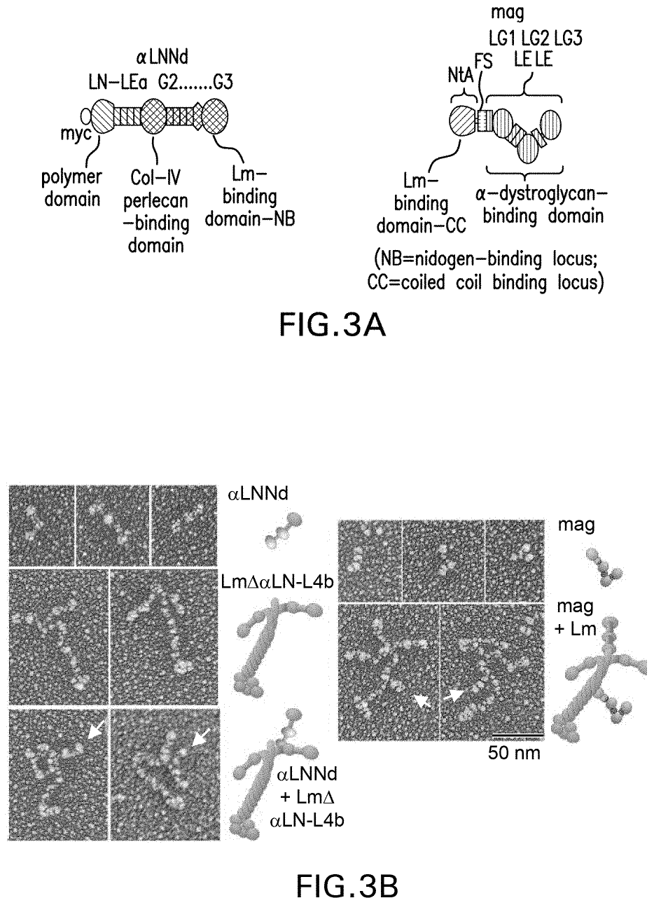

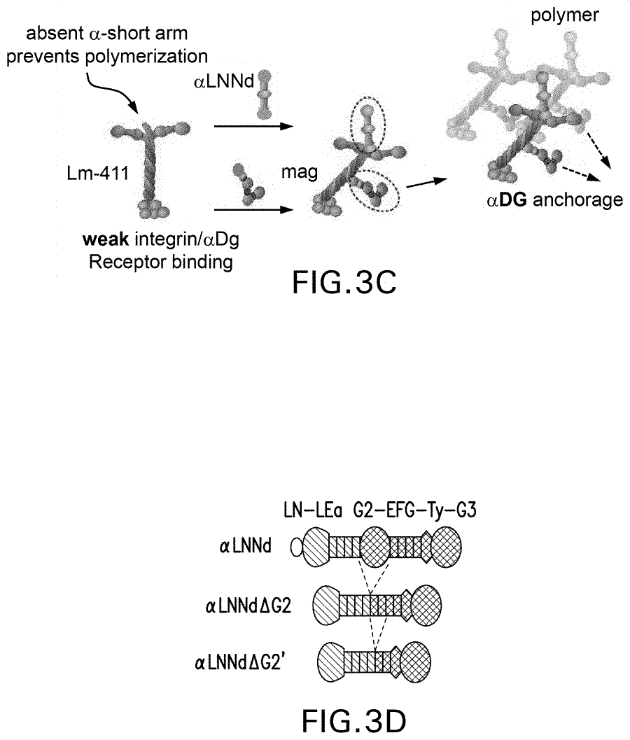

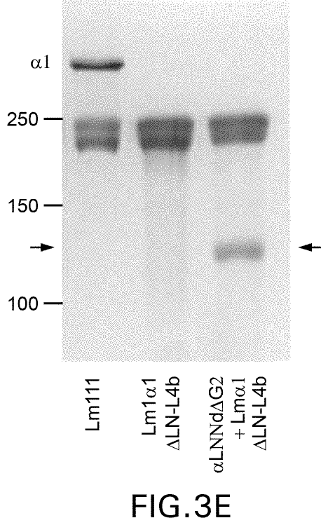

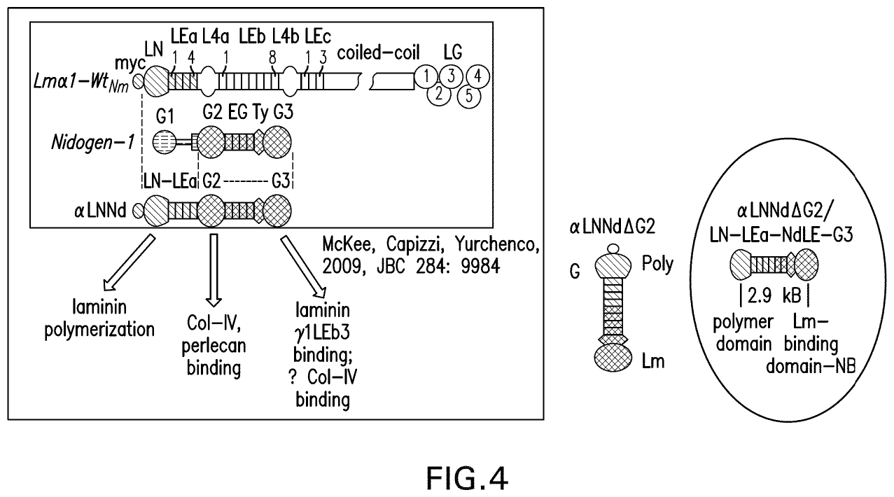

Recombinant laminins and chimeric linker proteins can repair basement membrane defects in models of LAMA2-MD. Recent advances in understanding the requirements for BM assembly have shown that laminin-binding proteins may provide an alternative arm for polymerization in a laminin that lacked an LN domain. αLNNd, βLNNd and γLNNd linker proteins can enable polymerization in laminins that lacked the corresponding αLN, βLN and γLN domains. See, McKee et al., Matrix Biol (2018) www.//doi.org/10.1016/j.matbio.2018.01.012, Chimeric protein identification of dystrophic, Pierson and other laminin polymerization residues. αLNNd consists of three globular domains with intervening rods resulting from the fusion of the Lmα1 LN-Lea domains with the nidogen-1 G2-G3 domains, shown in FIG. 3A and FIG. 4. The LN globular domain is a polymerization domain. G2 binds to collagen-IV and perlecan while G3 binds to the Lmγ1-LEb3 domain, creating an artificial arm that is attached to a locus near the short arm cross intersection. When bound to non-polymerizing laminin lacking the α-LN domain, a LNNd enables polymerization and collagen-IV recruitment to BMs, with no adverse effect on WT laminin. See, McKee, et al., J Biol Chem, (2009) 284(13):8984-8994.

Transgenic expression of αLNNd has been shown to ameliorate the dy2J muscular dystrophy and that, in combination with minagrin, a protein that enhanced receptor binding, also ameliorated the more severe dyW dystrophy. See, McKee et al., J Clin Invest (2017) 127(3) 1075-1089; Reinhard, et al., Sci Transl Med (2017) 9(396). Of additional note, it may be possible to treat patients with Pierson syndrome resulting from failures of laminin self-assembly by using βLNNd instead of αLNNd proteins to restore polymerization to glomerular Lm521 bearing β2LN mutations.

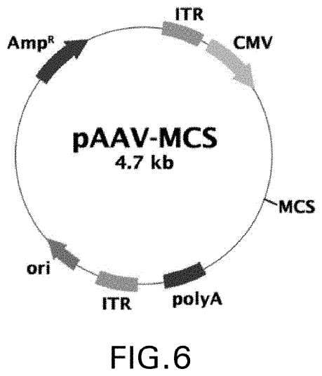

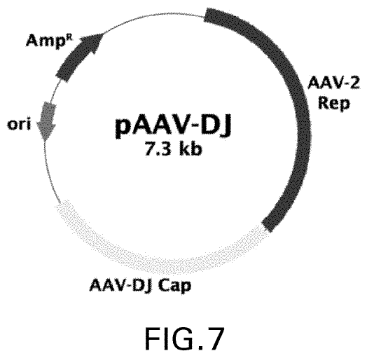

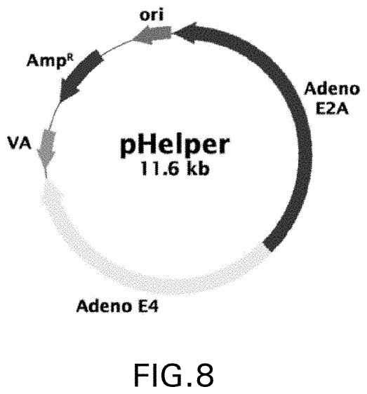

Adeno-associated virus (AAV) is one of the most promising of the gene delivery systems in which high expression can be achieved in muscle, peripheral nerve and other tissue. Potential risks include host cellular immune responses to transgene products and AAV capsid with subsequent loss of protein. However, this problem has been reduced by avoiding the creation of transgene neoantigens. The domains of αLNNd, βLNNd and γLNNd linker proteins are normally expressed as parts of larger basement membrane proteins, even in the dystrophic state, and are unlikely to be immunogenic. In order to take advantage of recent improvements in AAV delivery in which the CMV promoter has been enhanced, and with the largest insert capacity, the preferred AAV system for the present invention is the AAV-DJ system that employs an enhanced CMV promoter with a mixed serotype capsid and allows up to a 3.1 kB insert (Cell Biolabs, Inc., San Diego, Calif.) (see FIGS. 6-8).

A problem for AAV somatic gene expression of αLNNd is that while αLNNd is small enough to be expressed by AAV, the promoter would have to be very small and would be unlikely to provide good expression. A potential solution to this problem would be to reduce the size of the αLNNd DNA, which is 4.17 kB, so it could fit into AAV, but the concern was that reducing the size could affect the function of the protein for basement membrane assembly and myelination. Since the N- and C-terminal domains are essential, the focus was on reducing the size of the internal domains. The first modified protein that was made and designated αLNNdΔG2 is shown in FIGS. 3A and 4. Removal of G2 gave most of the needed reduction, but at the expense of losing direct coupling of the polymerizing laminin to collagen-IV and perlecan. Experiments conducted with Schwann cells, myotubes, and dorsal root ganglia revealed that G2 and its flanking LE/EGF-like domains to 3 kB were expendable so long as some nidogen-1 was present in the test system. Other experiments with transgenesis showed that substantial nidogen-1 remains in the basement membrane, indicating that size reduction of the αLNNd linker protein could be pursued. The present invention provides a new αLNNd linker protein designated αLNNdΔG2′ in which the internal G2 and two EGF-like spacer domains have been removed, reducing the size of the nucleotide sequence to about 2.9-3.0 kB, making it small enough to be expressed by AAV yet retaining the function of the protein for basement membrane assembly and myelination.

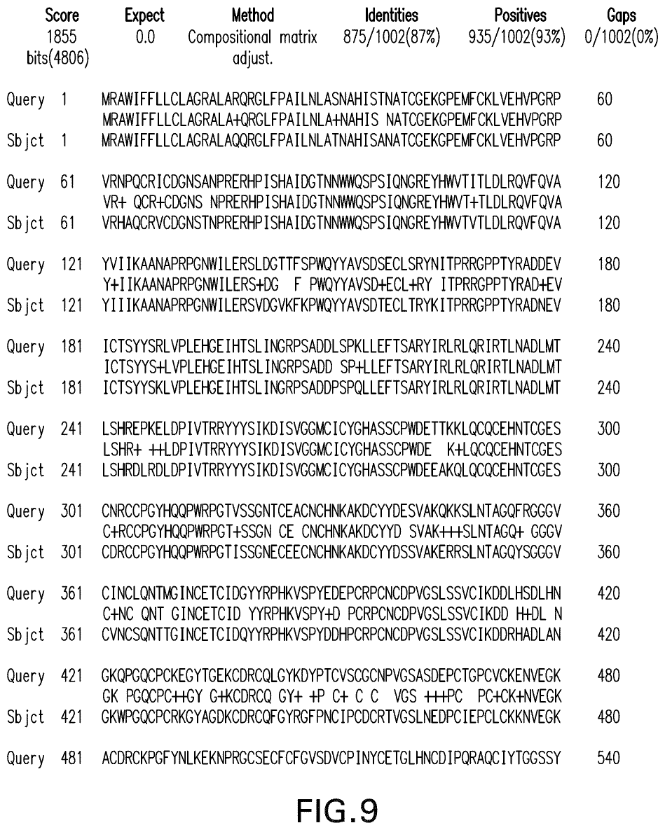

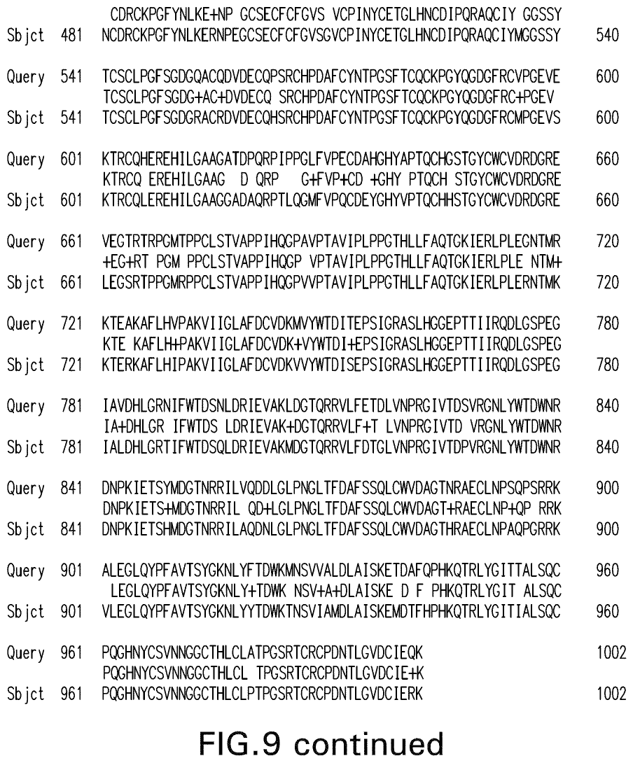

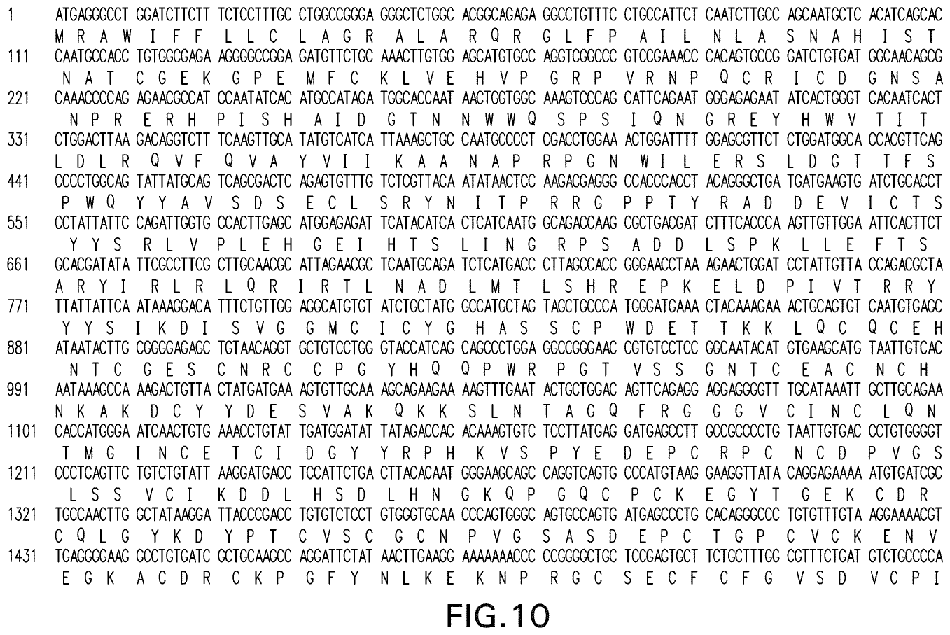

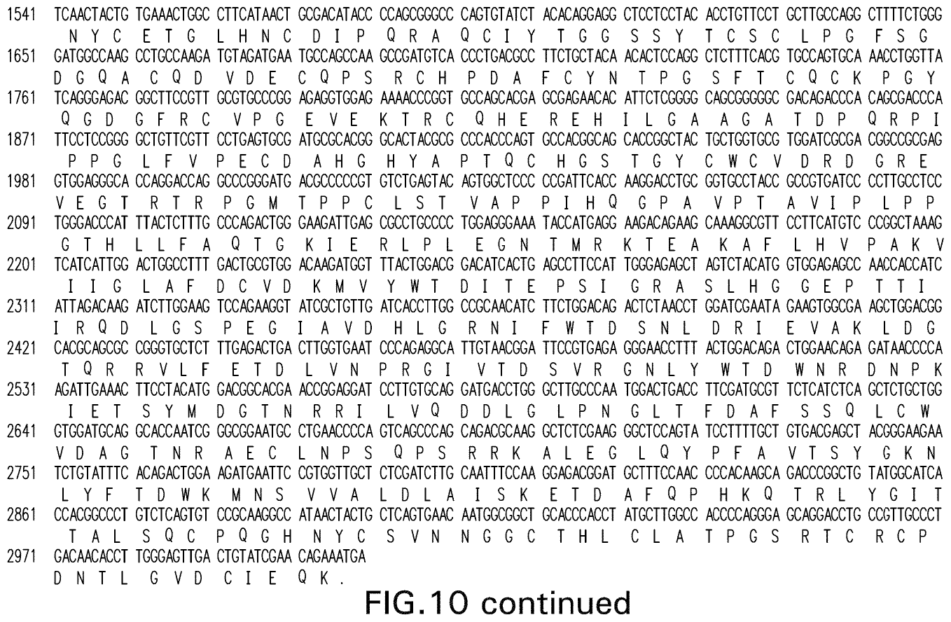

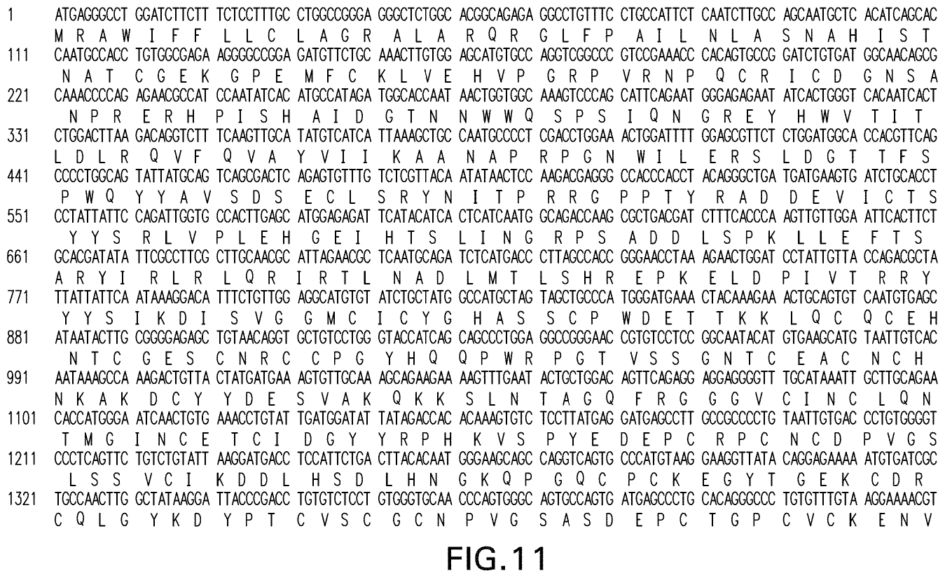

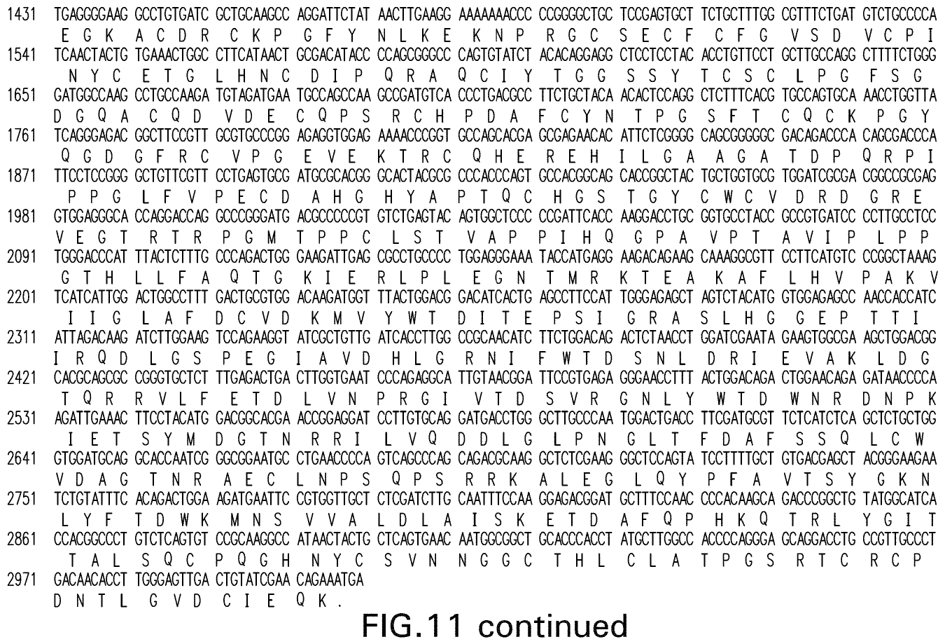

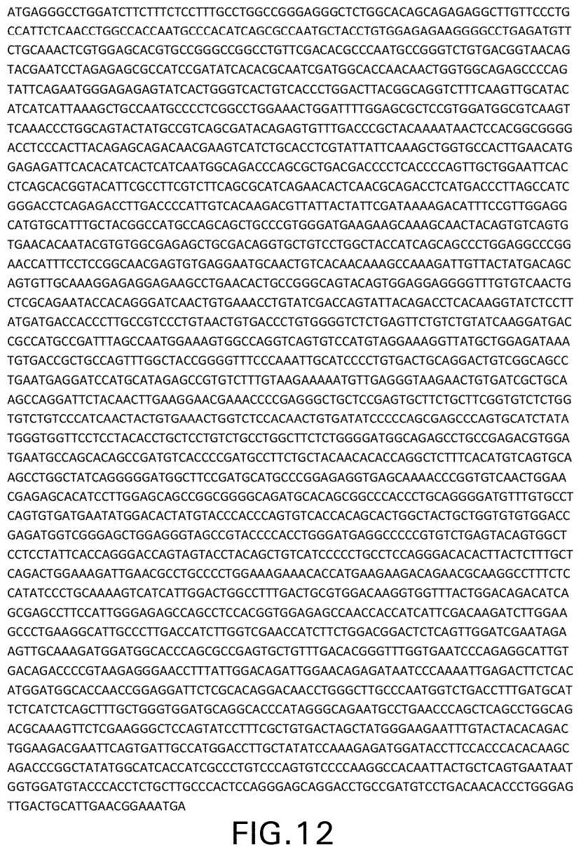

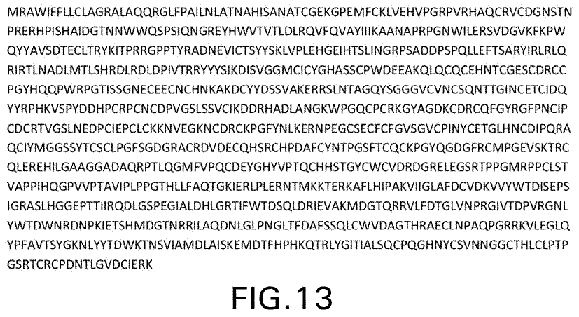

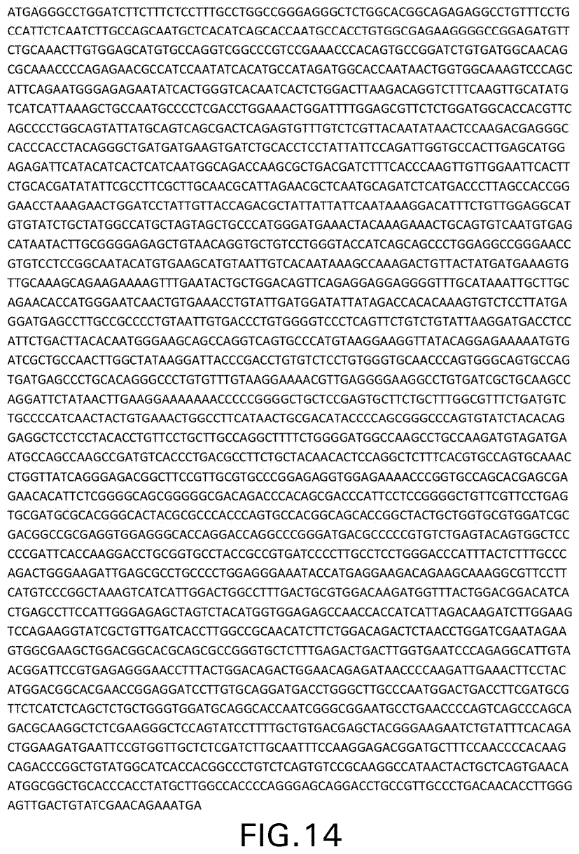

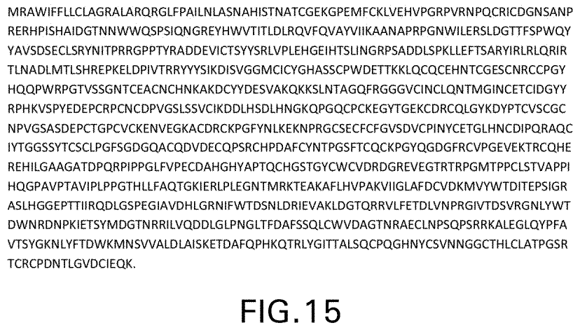

The present invention relates to using AAV-DJ-αLNNdΔG2′ constructs to restore laminin polymerization and basement membrane assembly in muscle, peripheral nerve and other tissue and ameliorate LAMA2-MD. It is expected that such methods and AAV-DJ-αLNNdΔG2′ constructs can be effective treatments for the human disease. For ease of reference, the vector constructs described herein are referred to as various AAV-DJ-αLNNdΔG2′ constructs, which indicate AAV-DJ constructs comprising nucleic acid sequences that encode mouse alphaLNNdDeltaG2short protein, among other elements. The human alphaLLNdDeltaG2short protein has an 87% identity with mouse alphaLLNdDeltaG2short protein, as shown in FIG. 9. It is expected that codon-optimized human constructs will function in the same desired manner to restore laminin polymerization and basement membrane assembly in muscle, peripheral nerve and other tissue and ameliorate LAMA2-MD. It is believed that patients with Pierson syndrome can be treated using the same AAV-DJ constructs by replacing the alpha1 segment with a beta1 segment from βLNNd protein in order to restore polymerization to glomerular Lm521 bearing β2LN mutations.

AAV-Compatible Laminin-Linker Protein alphaLNNdDeltaG2Short

### Abbreviations

AAV: adeno-associated virus
rAAV recombinant adeno-associated virus or viral vector
BM: basement membrane
αLNNd alpha laminin N-terminal domain linking protein
αLNNdΔG2′ alpha laminin N-terminal domain delta G2 short linking protein, alphaLNNdDeltaG2short
α-DG α-dystroglycan
βLNNdΔG2′ beta laminin N-terminal domain delta G2 short linking protein, betaLNNdDeltaG2short
ECM extracellular matrix
γLNNdΔG2′ gamma laminin N-terminal domain delta G2 short linking protein, gammaLNNdDeltaG2short
LE domain laminin-type epidermal growth factor-like domain
LG domain laminin G-like domain
LM or Lm laminin
LN domain laminin N-terminal domain

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., (catalytic activity)/(mg protein), or (immunological activity)/(mg protein), concentration in a biological compartment, or the like. “Activity” may refer to modulation of components of the innate or the adaptive immune systems.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human, including a human patient.

“alphaLNNd” (αLNNd) is a linker protein consisting of three globular domains with intervening rods resulting from the fusion of the Lmα1 LN-LEa domains with the nidogen-1 G2-G3 domains. The LN globular domain is a polymerization domain. G2 binds to collagen-IV and perlecan while G3 binds to the Lmα1-LEb3 domain, creating an artificial arm that is attached to a locus near the short arm cross intersection. When bound to non-polymerizing laminin lacking the αLN domain, αLNNd enables polymerization and collagen-IV recruitment to BMs, with no adverse effect on WT laminin.

“Treat” or “treating” means to administer a therapeutic agent, such as a composition containing any of the rAAV constructs of the present invention, internally or externally to a subject or patient having one or more disease symptoms, or being suspected of having a disease or being at elevated at risk of acquiring a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses transfection of any of the rAAV constructs or related methods of the present invention as applied to a human or animal subject, a cell, tissue, physiological compartment, or physiological fluid.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

### Recombinant AAVs

In some aspects, the invention provides isolated AAVs. As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, a rAAV having a capsid appropriate for the tissue being targeted can be selected.

For targeting the desired tissue in the context of treating laminin alpha-2 deficiency, one preferred rAAV is a combination of AAV-DJ capsid and AAV-2 Rep gene backbone, resulting in the various rAAV's described herein (See the sequence listing). Another preferred rAAV is AAV-9, a variant whose tissue expression pattern includes muscle and nerve.

Methods for obtaining recombinant AAVs having a desired capsid protein have been described (See, for example, US 2003/0138772, the contents of which are incorporated herein by reference in their entirety). A number of different AAV capsid proteins have been described, for example, those disclosed in G. Gao, et al., J. Virol, 78(12):6381-6388 (June 2004); G. Gao, et al, Proc Natl Acad Sci USA, 100(10):6081-6086 (May 13, 2003); US 2003-0138772, US 2007/0036760, US 2009/0197338 the contents of which relating to AAVs capsid proteins and associated nucleotide and amino acid sequences are incorporated herein by reference. For the desired packaging of the presently described constructs and methods, the AAV-9 vector and capsid and the AAV-DJ vector and capsid (SEQ ID NO: 17) are preferred. Further, other AAV vectors and capsids, some as yet not developed, may prove useful in the future as well. Typically, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., K. Fisher et al, J. Virol., 70:520-532 (1993) and U.S. Pat. No. 5,478,745.

In some embodiments, recombinant AAVs may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present invention include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA) from a transcribed gene.

### Recombinant AAV Vectors

“Recombinant AAV (rAAV) vectors” described herein are typically composed of, at a minimum, a transgene (e.g., encoding αLNNdΔG2′) and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product of interest (e.g., αLNNdΔG2′). The nucleic acid coding sequence is operatively linked to regulatory components in a manner which permits transgene transcription, translation, and/or expression in a cell of a target tissue.

The AAV sequences of the vector may comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, “Molecular Cloning. A Laboratory Manual”, 2d ed., Cold Spring harbor Laboratory, New York (1989); and K. Fisher et al., J. Virol., 70:520 532 (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA).

For nucleic acids encoding proteins, a polyadenylation sequence generally is inserted following the transgene sequences and before the 3′ AAV ITR sequence. An rAAV construct useful in the present invention may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available (see, e.g., Sambrook et al, and references cited therein at, for example, pages 3.18 3.26 and 16.17 16.27 and Ausubel et al., Current Protocols in Molecular Biology, John Wiley & Sons, New York, 1989). In some circumstances, a Foot and Mouth Disease Virus 2A sequence may be included in a polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the 13-actin promoter, the phosphoglycerol kinase (PGK) promoter, and the EF1a promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. IDSA, 88:5611-5 (1991)), and the neuron-specific vgf gene promoter (Piccioli et al., Neuron, 15:373-84 (1995)). In some embodiments, the tissue-specific promoter is a promoter of a gene selected from: neuronal nuclei (NeuN), glial fibrillary acidic protein (GFAP), adenomatous polyposis coli (APC), and ionized calcium-binding adapter molecule 1 (Iba-1). In some embodiments, the promoter is a CMV promoter. In some embodiments, the regulatory sequence is woodchuck hepatitis posttranscriptional regulatory element (WPRE) (Choi et al., Molecular Brain, 7:17-27 (2014)). SEQ ID NO: 177 provides the nucleotide sequence of WPRE. Modified forms of WPRE can also be used for transgene expression (Choi et al., Molecular Brain, 7:17-27 (2014)). In some embodiments, a truncated WPRE having the nucleotide sequence of SEQ ID NO:178 is used. In some embodiments, the promoter is a CBh general expression promoter, which derived from the CBA promoter that is commercially available from Vector Builder, Inc. (Chicago, Ill., USA) (Gray et al., Hum Gene Ther., 22:1143-1153 (2011)).

### Transgene Coding Sequences

The composition of the transgene sequence of a rAAV vector will depend upon the use to which the resulting vector will be put. For example, one type of transgene sequence includes a reporter sequence, which upon expression produces a detectable signal. In another example, the transgene encodes a therapeutic αLNNdΔG2′ protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In some aspects, the invention provides rAAV vectors for use in methods of preventing or treating a LAMA2 gene defect (e.g., heritable gene defects, somatic gene alterations) in a mammal, such as for example, a gene defect that results in a laminin alpha-2 polypeptide deficiency in a subject, and particularly for treating or reducing the severity or extent of deficiency in a subject manifesting a laminin alpha-2 deficiency. In some embodiments, methods involve administration of a rAAV vector that encodes one or more therapeutic peptides, polypeptides, shRNAs, microRNAs, antisense nucleotides, etc. in a pharmaceutically-acceptable carrier to the subject in an amount and for a period of time sufficient to treat the LAMA2 disorder in the subject having or suspected of having such a disorder.

### Recombinant AAV Administration

rAAVS are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, intratumoral, and other parental routes of administration. Routes of administration may be combined, if desired.

Delivery of certain rAAVs to a subject may be, for example, by administration into the bloodstream of the subject. Administration into the bloodstream may be by injection into a vein, an artery, or any other vascular conduit. Moreover, in certain instances, it may be desirable to deliver the rAAVs to brain tissue, meninges, neuronal cells, glial cells, astrocytes, oligodendrocytes, cerebrospinal fluid (CSF), interstitial spaces and the like. In some embodiments, recombinant AAVs may be delivered directly to the spinal cord or brain with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intracerebrally, intrathecally, intracerebrally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs.

### Recombinant AAV Compositions

The rAAVs may be delivered to a subject in compositions according to any appropriate methods known in the art. The rAAV, preferably suspended in a physiologically compatible carrier (e.g., in a composition), may be administered to a subject, e.g., a human, mouse, rat, cat, dog, sheep, rabbit, horse, cow, goat, pig, guinea pig, hamster, chicken, turkey, or a non-human primate (e.g., Macaque). In certain embodiments, compositions may comprise a rAAV alone, or in combination with one or more other viruses (e.g., a second rAAV encoding having one or more different transgenes).

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration, the level of gene or RNA expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In certain embodiments, 10¹² rAAV genome copies per subject is effective to target CNS tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pII adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

General Methods Relating to Delivery of rAAV Compositions

The present invention provides stable pharmaceutical compositions comprising rAAV virions. The compositions remain stable and active even when subjected to freeze/thaw cycling and when stored in containers made of various materials, including glass.

Recombinant AAV virions containing a heterologous nucleotide sequence of interest can be used for gene delivery, such as in gene therapy applications, for the production of transgenic animals, in nucleic acid vaccination, ribozyme and antisense therapy, as well as for the delivery of genes in vitro, to a variety of cell types.

Generally, rAAV virions are introduced into the cells of a subject using either in vivo or in vitro transduction techniques. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant AAV virions with the cells e.g., in appropriate media, and screening for those cells harboring the DNA of interest using conventional techniques such as Southern blots and/or PCR, or by using selectable markers. Transduced cells can then be formulated into pharmaceutical compositions, described more fully below, and the composition introduced into the subject by various routes, such as by intramuscular, intravenous, intra-arterial, subcutaneous and intraperitoneal injection, or by injection into smooth muscle, using e.g., a catheter, or directly into an organ.

For in vivo delivery, the rAAV virions will be formulated into a pharmaceutical composition and will generally be administered parenterally, e.g., by intramuscular injection directly into skeletal muscle, intra-articularly, intravenously or directly into an organ.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art.

Thus, a “therapeutically effective amount” will fall in a relatively broad range that can be determined through clinical trials. For example, for in vivo injection, i.e., injection directly to the subject, a therapeutically effective dose will be on the order of from about 10⁵ to 10¹⁶ of the rAAV virions, more preferably 10⁸ to 10¹⁴ rAAV virions. For in vitro transduction, an effective amount of rAAV virions to be delivered to cells will be on the order of 10⁵ to 10¹³, preferably 10⁸ to 10¹³ of the rAAV virions. If the composition comprises transduced cells to be delivered back to the subject, the amount of transduced cells in the pharmaceutical compositions will be from about 10⁴ to 10¹⁰ cells, more preferably 10⁵ to 10⁸ cells. The dose, of course, depends on the efficiency of transduction, promoter strength, the stability of the message and the protein encoded thereby, etc. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

As used herein, “polymerase chain reaction” or “PCR” refers to a procedure or technique in which specific nucleic acid sequences, RNA and/or DNA, are amplified as described in, e.g., U.S. Pat. No. 4,683,195. Generally, sequence information from the ends of the region of interest or beyond is used to design oligonucleotide primers. These primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

### Nucleic Acids

The invention also comprises certain constructs and nucleic acids encoding the αLNNdΔG2′ protein described herein. Certain constructs and sequences, including selected sequences listed in the sequence listing including SEQ ID NO: 1 and SEQ ID NO: 24 may be useful in embodiments of the present invention.

Preferably, the nucleic acids hybridize under low, moderate or high stringency conditions, and encode an αLNNdΔG2′ protein that maintains biological function. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook, et al., supra). The conditions of temperature and ionic strength determine the “stringency” of the hybridization. Typical low stringency hybridization conditions include 55° C., 5×SSC, 0.1% SDS and no formamide; or 30% formamide, 5×SSC, 0.5% SDS at 42° C. Typical moderate stringency hybridization conditions are 40% formamide, with 5× or 6×SSC and 0.1% SDS at 42° C. High stringency hybridization conditions are 50% formamide, 5× or 6×SSC at 42° C. or, optionally, at a higher temperature (e.g., 57° C., 59° C., 60° C., 62° C., 63° C., 65° C. or 68° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, e.g., oligonucleotides, the position of mismatches becomes more important, and the length of the oligonucleotide determines its specificity (see Sambrook, et al., supra, 11.7-11.8).

The αLNNdΔG2′ mouse polypeptide comprises the amino acid sequence of SEQ ID NO: 21. The αLNNdΔG2′ human polypeptide comprises the amino acid sequence of SEQ ID NO: 22 and has an 87% identity with the mouse polypeptide as shown in FIG. 9. αLNNdΔG2′ polypeptides comprising amino acid sequences that are at least about 90% identical and most preferably at least about 95% identical (e.g., 95%, 96%, 97%, 98%, 99%, 100%) to the αLNNdΔG2′ amino acid sequences provided herein (e.g., SEQ ID NOs: 21-22) are contemplated with respect to restoring laminin polymerization function, when the comparison is performed by a BLAST algorithm wherein the parameters of the algorithm are selected to give the largest match between the respective sequences over the entire length of the respective reference sequences. Polypeptides comprising amino acid sequences that are at least about 90% similar and most preferably at least about 95% similar (e.g., 95%, 96%, 97%, 98%, 99%, 100%) to any of the reference αLNNdΔG2′ amino acid sequences when the comparison is performed with a BLAST algorithm wherein the parameters of the algorithm are selected to give the largest match between the respective sequences over the entire length of the respective reference sequences, are also included in constructs and methods of the present invention.

Sequence identity refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared ×100. For example, if 6 of 10 of the positions in two sequences are matched or homologous when the sequences are optimally aligned then the two sequences are 60% homologous. Generally, the comparison is made when two sequences are aligned to give maximum percent homology.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

This invention also provides expression vectors comprising various nucleic acids, wherein the nucleic acid is operably linked to control sequences that are recognized by a host cell when the host cell is transfected with the vector. Also provided are the virions comprising recombinant AAV-DJ and certain AAV-2 sequences, as well as nucleic acid sequences for expressing αLNNdΔG2′ under the direction of a CMV promoter and a CMV enhancer. Alternative promoters may be used provided that they are small in size and have high activity with good expression. Within these constructs, the rAAV2 sequences correspond to the 5′ and 3′ ITR sequences, e.g., SEQ ID NOS: 11 and 16 and others as described in the sequence listing). These sequences were packaged with the AAV-DJ capsid to form the virions that are therapeutic to laminin alpha-2 deficiency in the present invention.

### Pharmaceutical Compositions and Administration

To prepare pharmaceutical or sterile compositions of the compositions of the present invention, the AAV-DJ vectors or related compositions may be admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In an embodiment of the invention, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the composition or therapeutic can be administered by an invasive route such as by injection (see above). In further embodiments of the invention, the composition, therapeutic, or pharmaceutical composition thereof, is administered intravenously, subcutaneously, intramuscularly, intraarterially, intra-articularly (e.g., in arthritis joints), intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

Alternately, one may administer the AAV-DJ vector or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the brain. The liposomes will be targeted to and taken up selectively by the desired tissue.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of a rAAV-DJ-αLNNdΔG2′ based compound of the invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Kits

The present invention also provides kits comprising the components of the combinations of the invention in kit form. A kit of the present invention includes one or more components including, but not limited to, rAAV-DJ-αLNNdΔG2′ based compound, as discussed herein, in association with one or more additional components including, but not limited to a pharmaceutically acceptable carrier and/or a chemotherapeutic agent, as discussed herein. The rAAV-DJ-αLNNdΔG2′ based compound or composition and/or the therapeutic agent can be formulated as a pure composition or in combination with a pharmaceutically acceptable carrier, in a pharmaceutical composition.

In one embodiment, a kit includes an rAAV-DJ-αLNNdΔG2′ based compound/composition of the invention or a pharmaceutical composition thereof in one container (e.g., in a sterile glass or plastic vial) and a pharmaceutical composition thereof and/or a chemotherapeutic agent in another container (e.g., in a sterile glass or plastic vial).

In another embodiment of the invention, the kit comprises a combination of the invention, including an rAAV-DJ-αLNNdΔG2′ based compound, along with a pharmaceutically acceptable carrier, optionally in combination with one or more chemotherapeutic agent component formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### GENERAL METHODS

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

### EXAMPLES

### Example 1

### AlphaLNNdDeltaG2short (αLNNdΔG2′) Construct Development

Removal of the G2 nidogen-1 domain in αLNNd pcDNA3.1 Zeo was accomplished with overlapping PCR. In the first round of PCR, a 1.2 Kb-5′ (F1noG2 1F 5′-ctgggtcactgtcaccctgg-3′ (SEQ ID NO: 2) and noG2 2R 5′-atggattctgaagacagacaccagagacac-3′ (SEQ ID NO: 3)) and 1.8 Kb-3′ (no G2 2F 5′-ctggtgtctgtcttcagaatccatgctac-3′ (SEQ ID NO: 4) and F1 no G2 1R 5′-gaaggcacagtcgaggctgatcag-3′ (SEQ ID NO: 5)) product was generated on either side of the G2 nidogen-1 domain of αLNNd. They were sewn together with a second round of PCR (F1noG2 1F and F1 no G2 1R) into a 3 Kb product which was then digested with EcoRI to 2.4 Kb and ligated into the 5.85 Kb EcoRI αLNNd pcDNA 3.1 zeo vector (generating an 8.25 Kb noG2 αLNNd pcDNA3.1 zeo plasmid). A further 2 EGF (270 bp) deletion of noG2 αLNND was performed with overlapping PCR primers (Bam shnoG2 1F 5′-cggcagcctgaatgaggatccatgcataga-3′ (SEQ ID NO: 6) and shnoG2 2R 5′-cacagtagttgatgggacagacacc-3′ (SEQ ID NO: 7)) and 3′ (shnoG2 2F 5′-gtctctggtgtctgtcccatcaacta-3′ (SEQ ID NO: 8) and sse shnoG2 1R 5′-gaggcacaaacatcccctgcagggtgggcc-3′ (SEQ ID NO: 9) to generate 160 bp and 357 bp products, respectively. After a second round of PCR, a 485 bp BamHI-SbfI digested insert was ligated into a likewise digested noG2 αLNNd pcDNA3.1 zeo vector (7.5 Kb). To remove the N-terminal Myc tag on the short no G2 αLNNd open reading frame (ORF), a 1.5 Kb BamHI insert was moved from the F3-8 mck-pA construct to the MCS-AAV vector (4.6 Kb Cell Biolabs, VPK-410-DJ) generating a 6.1 Kb AAV-5′F1 no tag-10 plasmid. The short noG2 αLNND pcDNA3.1 zeo plasmid was digested with FseI and XhoI to generate a 2.8 Kb insert which was ligated into the similarly digested AAV-5′F1 no tag-10 vector (4.9 Kb). The final vector size was 7.7 Kb with an ORF for alphaLNNdDeltaG2short (αLNNdΔG2′) of 3009 bp (SEQ ID NO: 1).

### Example 2

### Generation of AAV Virus

The αLNNdΔG2′-MCS plasmid was triple transfected along with AAV-DJ pHelper pHelper plasmids (SEQ ID NOS: 1, 17, 20, respectively; FIGS. 6-8) (Cell Biolabs, Inc., San Diego, Calif.) into adherent HEK293 in a 1:1:1 ratio using a common method of calcium phosphate transient transfection. Briefly, 12.5 ug each/150 mm dish (10-150 mm dishes per prep) were added to the 75% confluent HEK293 cells overnight according to manufacturer's instructions (Sigma-Aldrich Corp., St. Louis, Mo., catalog #CAPHOS). Virus was harvested from the cultures 96 hours later with an AAVpro purification kit (Takara Bio USA, Inc., Mountain View, Calif., catalog #6666). Alternative methods of purification are available including freeze-thaw or Triton-100 lysis of cells followed by PEG8000 and/or cesium chloride centrifugation. Viral titer was determined with real time PCR (AAVpro titration kit, Takara Bio USA, Inc., Mountain View, Calif., catalog #6233).

### Example 3

### Expression and Analysis of AAV-generated αLNNdΔG2′ Protein

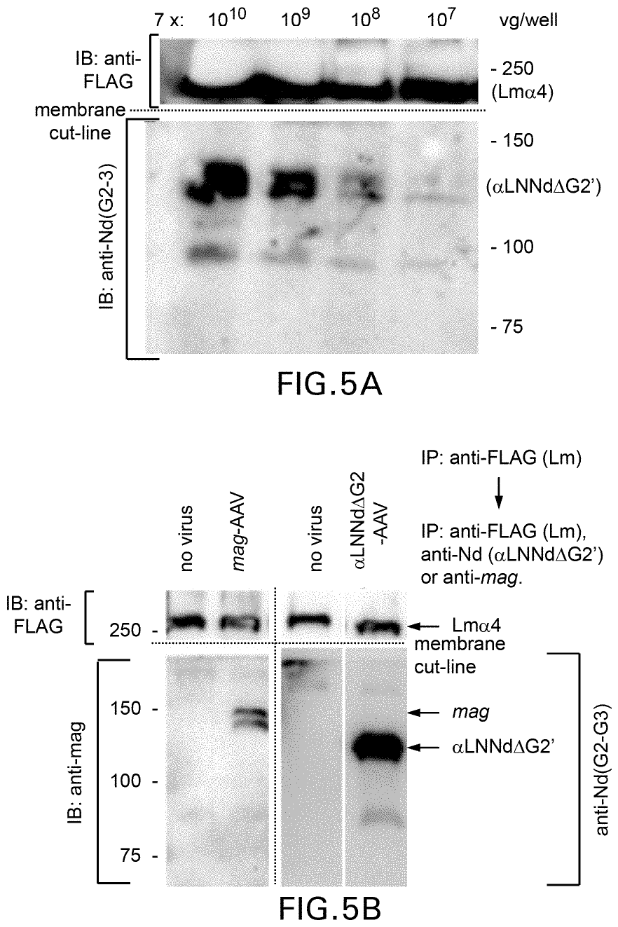

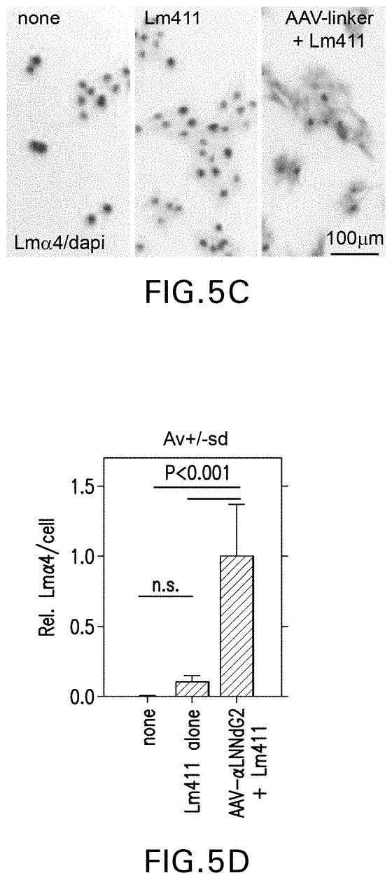

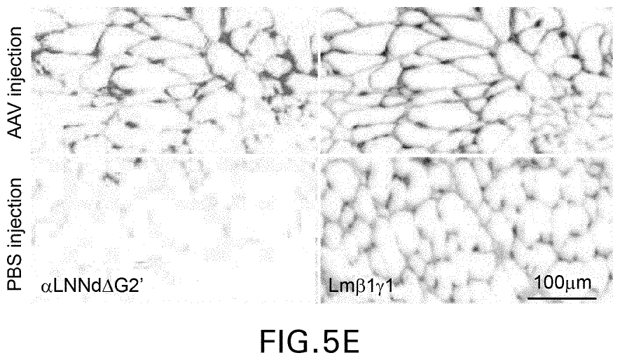

Stably transfected 411 HEK293 cells were infected with approximately 6×10 vg/6-wells dish. Four days later, the conditioned media was evaluated by immunoprecipitation with α-flag agarose beads for 1 hour at room temperature, followed by western blot analysis. Western blots were cut and stained with anti-flag (top) or anti-G2-G2 nidogen (bottom) at 1 μg/ml. Results are shown in FIG. 5A. Additionally, the conditioned AAV 411 HEK293 media was added to high passage rat Schwann cells for 1 hr and analyzed by immunofluorescence for 411 laminin assembly using 1 ug/ml chicken anti-α4 and 1:100 anti-chicken Alexa Fluor 647 (Life Technologies, Carlsbad, Calif., catalog #A-21449). A substantial increase of Lm411 assembly resulted from the AAV-generated αLNNdΔG2′ protein, shown in FIGS. 5C and 5D.

AAVαLNNdΔG2′ (virus, 10¹⁰ vg in ˜25 μl) or PBS buffer was injected i.m. into a 1-week old dy3K/dy3K mag mouse. Two week later, the quadriceps were harvested, sectioned, and stained with antibody to detect αLNNdΔG2′ (red) and laminins (green), shown in FIG. 5E. The ∞1LN epitope of αLNNdΔG2′ was detected in the quadriceps muscle tissue, indicating the linker was incorporated into the muscle sarcolemma.

### Example 4

### Restoring Laminin α2 to Symptomatic Mice

Injection of AAV-DJ-αLNNdΔG2′ constructs in dy3K/dy3K mice expressing a mag transgene, a miniaturized version of agrin FIG. 3B (SEQ ID NO: 23) and injection of AAV-DJ-αLNNdΔG2′ construct in dy3K/dy3K mice expressing the αLNNd transgene are done to evaluate one virus infection at a time in conjunction with stable and already characterized expression of the paired linker protein and to validate each linker protein separately, minimizing variability. The initial analysis is on muscle to determine which muscles are populated with αLNNdΔG2′ and mag following the extent of nerve expression, and the persistence of expression following injection, using immunofluorescence microscopy with specific linker and laminin antibodies described in McKee, et al., (2017) J Clin Invest 127(3):1075-1089; Reinhard, et al., (2017) Sci Transl Med 9(396).

Following assessment of the initial analysis, dy3K/dy3K mice are co-infected with both virus preparations. Injections will be given post-natal day 1 or 2, given the perinatal time course of myelination (SC proliferation commencing before birth by radial sorting occurring substantially in the first post-natal week). Phenotype and histology analyses to be done include (1) measurements of measure survival, body weights, muscle weights, time on vertical grids, grip strength and overall behavior at different ages; (2) examination of diaphragm, intercostal muscles and phrenic nerve; (3) skeletal muscle analysis by H&E and Sirius Red (collagen)-stained histology of forelimb extensor carpi radialis and diaphragm/intercostal muscles at different ages with morphometric quantitation of fiber size, number, regeneration (fraction of myofibers with central nuclei), inflammation and fibrosis; (4) peripheral nerve analysis by examining immunostained nerve and roots to estimate the extent of linker-protein expression and to detect relative changes in laminin subunits; examine methylene-blue stained semi-thin sections using electron microscopy to quantitatively evaluate the extent of axonal sorting, myelination, myelin thickness, and fraction of naked axons; determine SC proliferation from EdU/dapi ratios, and using qRT-PCR to evaluate maturation of myelination (e.g., Oct6, Sox2, cJun).

Results of the analysis are used to optimize delivery and evaluate variants of the αLNNdΔG2′ and mag linker proteins that may further improve functions.

### Example 5

### Expression of αLNNdΔG2′ with AAV with a Variant Serotype Capsid

The αLNNdΔG2′ DNA is inserted into an AAV vector with coding for a different capsid serotype or composite serotype for the purpose of altering tissue specificity, e.g. only skeletal muscle plus heart or predominantly liver. Note: αLNNdΔG2′ is a soluble secreted protein in which the site of synthesis need not be the target cell type.

### Example 6

### AAV Capsid Sequence Modified to Reduce Ubiquitination

AAV-DJ, like other AAV, contain several phosphorylation and ubiquitination sites on the capsid. Point mutations on the rep/cap plasmid at K137R, S503A, and T251A were found to substantially increase protein expression in vitro and in vivo (described in Mao, Wang, Yan, Li, Wang and Li, 2016, “Single point mutation in adeno-associated viral vectors-DJ capsid leads to improvement for gene delivery in vivo. BMC Biotechnology 16: 1-8). The AAV plasmid can readily be modified to introduce this improvement.

### Example 7

### Expression of αLNNdΔG2′ with AAV Using a Specialized Promoter

The αLNNdΔG2′ DNA is inserted into an AAV vector with a different promoter/enhancer with the effect of (a) changing specificity and/or (b) increasing the allowable open reading frame of the insert. An example, used to drive expression of micro-dystrophin in skeletal muscle and heart, is the 436 bp CK8e promoter/enhancer that has been modified from the muscle creatine kinase gene basal promoter and upstream enhancer. The CK8e promoter/enhancer is described in J. N. Ramos et al., 2019, Molecular Therapy, 27: 623-635.

### Example 8

### Expression of Lmα1LNNdΔG2′ with Alternative Signal Sequence

The protein αLNNdΔG2′ and related proteins have been expressed in vitro and in mice using the BM-40 signal sequence, which has the nucleotide sequence in SEQ ID NO: 25 and has been given the letter code A in Table 2 below. An alternative is to express the protein with the endogenous α1 subunit signal peptide, which has the nucleotide sequence in SEQ ID NO: 27 and has been given the letter code A′ in Table 2.

Table 2 provides a list of all of the variant protein sequences with assigned letter codes that can be used with either the BM-40 signal peptide or the laminin endogenous signal peptide that normally precedes the laminin N-terminal subunit. These domains can be used to create linker proteins that enable laminin polymerization. Mouse domains of the laminin-binding linker protein and internally reduced-sized linker proteins that can enable polymerization have been assigned letter codes A, A′ to P for both nucleotide and amino acid sequences (SEQ ID NOS: 25-58). Alternative N-terminal domains, mouse and human, have been assigned letter codes Q to Z and a to b for both nucleotide and amino acid sequences (SEQ ID NOS: 59-106). Additional C-terminal domains, mouse and human non-neural agrin dystroglycan-binding domains that can be fused C-terminal (5′ to) to the nidogen laminin-binding G3 domain of polymerization linker proteins, have been assigned letter codes c to j for both nucleotide and amino acid sequences (SEQ ID NOS: 107-138).

Table 3 provides the mouse and human nucleotide and amino acid sequences for each of the variant protein sequences listed in Table 2 and provides the SEQ ID NO assigned to these sequences in the Sequence Listing.

TABLE 2
Domain Single Letter Codes

| Letter | | | | DNA |
|---|---|---|---|---|
| Code | Gene | Protein | Domain | size, bp⁶ |
| A | LAMA1 | Laminin-α1 | BM-40 signal | 51 |
| | | | peptide | |
| A′ | LAMA1 | Laminin-α1 | endogenous | 72 |
| | | | signal peptide | |
| B | LAMA1 | Laminin-α1 | LN | 753 |
| C | LAMA1 | Laminin-α1 | LEa-1 | 171 |
| D | LAMA1 | Laminin-α1 | LEa-2 | 210 |
| E | LAMA1 | Laminin-α1 | LEa-3 | 177 |
| F | LAMA1 | Laminin-α1 | LEa-4 | 168 |
| G | LAMA1 | Laminin-α1 | LF fragment | 33 |
| H | NID1 | Nidogen-1 | G2 | 843 |
| I | NID1 | Nidogen-1 | EGF-like-2 | 126 |
| J | NID1 | Nidogen-1 | EGF-like-3 | 126 |
| K | NID1 | Nidogen-1 | spacer betw. | 18 |
| | | | EGF-like 3 & 4 | |
| L | NID1 | Nidogen-1 | EGF-like-4 | 132 |
| M | NID1 | Nidogen-1 | EGF-like-5 | 141 |
| N | NID1 | Nidogen-1 | G3-TY | 282 |
| O | NID1 | Nidogen-1 | G3-Propeller | 744 |
| P | NID1 | Nidogen-1 | G3-EGF-like-6 | 171 |
| Q | LAMB1 | Laminin-β1 | signal peptide | 63 |
| R | LAMB1 | Laminin-β1 | LN | 744 |
| S | LAMB1 | Laminin-β1 | LEa-1 | 192 |
| T | LAMB1 | Laminin-β1 | LEa-2 | 189 |
| U | LAMB1 | Laminin-β1 | LEa-3 | 180 |
| V | LAMB1 | Laminin-β1 | LEa-4 | 156 |
| W | LAMC1 | Laminin-γ1 | signal peptide | 99 |
| X | LAMC1 | Laminin-γ1 | LN | 768 |
| Y | LAMC1 | Laminin-γ1 | LEa-1 | 168 |
| Z | LAMC1 | Laminin-γ1 | LEa-2 | 168 |
| a | LAMC1 | Laminin-γ1 | LEa-3 | 168 |
| b | LAMC1 | Laminin-γ1 | LEa-4 | 168 |
| c | AGRN | non-neural | LG spacer-1 | 27 |
| | | agrin | | |
| d | AGRN | non-neural | EGF-like 2 | 114 |
| | | agrin | | |
| e | AGRN | non-neural | EGF-like 3 | 117 |
| | | agrin | | |
| f | AGRN | non-neural | LG spacer-2 | 27 |
| | | agrin | | |
| g | AGRN | non-neural | LG2 | 537 |
| | | agrin | | |
| h | AGRN | non-neural | EGF-like 4 | 120 |
| | | agrin | | |
| i | AGRN | non-neural | LG spacer-2 | 30 |
| | | agrin | | |
| j | AGRN | non-neural | LG3 | 537 |
| | | agrin | | |

⁶Mouse bp number shown. Human bp same or similar.

TABLE 3
Domain Sequences

| SEQ | Domain | | |
|---|---|---|---|
| ID | Letter | | |
| NO | Code | Domain Name | Sequence |
| 25 | A | Mouse BM-40 (Sparc) | ATGAGGGCCTGGATCTTCTTTCTCCTTTGCCTGGCC |
| | | signal sequence [DNA, | GGGAGGGCTCTGGCA |
| | | 51 bp) | |
| 26 | A | Mouse BM-40 (Sparc) | MRAWIFFLLCLAGRALA |
| | | signal peptide | |
| 27 | A′ | Mouse Lm α1 | ATGCGCGGCAGCGGCACGGGAGCCGCGCTCCTGG |
| | | endogenous signal | TGCTCCTGGCCTCGGTGCTCTGGGTCACCGTGCGG |
| | | sequence [DNA, 72 bp] | AGC |
| 28 | A′ | Mouse laminin α1 | MRGSGTGAALLVLLASVLWVTVRS |
| | | endogenous signal | |
| | | peptide | |
| 29 | A′ | Laminin (Lm) α1 | ATGAGGGCCTGGATCTTCTTTCTCCTTTGCCTGGCC |
| | | signal peptide [DNA, | GGGAGGGCTCTGGCA |
| | | 51 bp] | |
| 30 | A′ | Human laminin α1 | MRAWIFFLLCLAGRALA |
| | | signal peptide | |
| 31 | B | Mouse Lm α1 LN | CAGCAGAGAGGCTTGTTCCCTGCCATTCTCAACCT |
| | | domain [DNA, 753 bp] | GGCCACCAATGCCCACATCAGCGCCAATGCTACCT |
| | | | GTGGAGAGAAGGGGCCTGAGATGTTCTGCAAACT |
| | | | CGTGGAGCACGTGCCGGGCCGGCCTGTTCGACAC |
| | | | GCCCAATGCCGGGTCTGTGACGGTAACAGTACGA |
| | | | ATCCTAGAGAGCGCCATCCGATATCACACGCAATC |
| | | | GATGGCACCAACAACTGGTGGCAGAGCCCCAGTA |
| | | | TTCAGAATGGGAGAGAGTATCACTGGGTCACTGTC |
| | | | ACCCTGGACTTACGGCAGGTCTTTCAAGTTGCATA |
| | | | CATCATCATTAAAGCTGCCAATGCCCCTCGGCCTG |
| | | | GAAACTGGATTTTGGAGCGCTCCGTGGATGGCGTC |
| | | | AAGTTCAAACCCTGGCAGTACTATGCCGTCAGCGA |
| | | | TACAGAGTGTTTGACCCGCTACAAAATAACTCCAC |
| | | | GGCGGGGACCTCCCACTTACAGAGCAGACAACGA |
| | | | AGTCATCTGCACCTCGTATTATTCAAAGCTGGTGC |
| | | | CACTTGAACATGGAGAGATTCACACATCACTCATC |
| | | | AATGGCAGACCCAGCGCTGACGACCCCTCACCCC |
| | | | AGTTGCTGGAATTCACCTCAGCACGGTACATTCGC |
| | | | CTTCGTCTTCAGCGCATCAGAACACTCAACGCAGA |
| | | | CCTCATGACCCTTAGCCATCGGGACCTCAGAGACC |
| | | | TTGACCCCATTGTCACAAGACGTTATTACTATTCG |
| | | | ATAAAAGACATTTCCGTTGGAGGC |
| 32 | B | Mouse Lm α1 LN | QQRGLFPAILNLATNAHISANATCGEKGPEMFCKLV |
| | | [polymerization | EHVPGRPVRHAQCRVCDGNSTNPRERHPISHAIDGT |
| | | domain] | NNWWQSPSIQNGREYHWVTVTLDLRQVFQVAYIIIK |
| | | | AANAPRPGNWILERSVDGVKFKPWQYYAVSDTECL |
| | | | TRYKITPRRGPPTYRADNEVICTSYYSKLVPLEHGEI |
| | | | HTSLINGRPSADDPSPQLLEFTSARYIRLRLQRIRTLN |
| | | | ADLMTLSHRDLRDLDPIVTRRYYYSIKDISVGG |
| 33 | B | Human Lm α1 LN | CGGCAGAGAGGCCTGTTTCCTGCCATTCTCAATCT |
| | | [DNA, 753 bp] | TGCCAGCAATGCTCACATCAGCACCAATGCCACCT |
| | | | GTGGCGAGAAGGGGCCGGAGATGTTCTGCAAACT |
| | | | TGTGGAGCATGTGCCAGGTCGGCCCGTCCGAAAC |
| | | | CCACAGTGCCGGATCTGTGATGGCAACAGCGCAA |
| | | | ACCCCAGAGAACGCCATCCAATATCACATGCCAT |
| | | | AGATGGCACCAATAACTGGTGGCAAAGTCCCAGC |
| | | | ATTCAGAATGGGAGAGAATATCACTGGGTCACAA |
| | | | TCACTCTGGACTTAAGACAGGTCTTTCAAGTTGCA |
| | | | TATGTCATCATTAAAGCTGCCAATGCCCCTCGACC |
| | | | TGGAAACTGGATTTTGGAGCGTTCTCTGGATGGCA |
| | | | CCACGTTCAGCCCCTGGCAGTATTATGCAGTCAGC |
| | | | GACTCAGAGTGTTTGTCTCGTTACAATATAACTCC |
| | | | AAGACGAGGGCCACCCACCTACAGGGCTGATGAT |
| | | | GAAGTGATCTGCACCTCCTATTATTCCAGATTGGT |
| | | | GCCACTTGAGCATGGAGAGATTCATACATCACTCA |
| | | | TCAATGGCAGACCAAGCGCTGACGATCTTTCACCC |
| | | | AAGTTGTTGGAATTCACTTCTGCACGATATATTCG |
| | | | CCTTCGCTTGCAACGCATTAGAACGCTCAATGCAG |
| | | | ATCTCATGACCCTTAGCCACCGGGAACCTAAAGA |
| | | | ACTGGATCCTATTGTTACCAGACGCTATTATTATT |
| | | | CAATAAAGGACATTTCTGTTGGAGGC |
| 34 | B | Human Lm α1 LN | RQRGLFPAILNLASNAHISTNATCGEKGPEMFCKLVE |
| | | | HVPGRPVRNPQCRICDGNSANPRERHPISHAIDGTNN |
| | | | WWQSPSIQNGREYHWVTITLDLRQVFQVAYVIIKAA |
| | | | NAPRPGNWILERSLDGTTFSPWQYYAVSDSECLSRY |
| | | | NITPRRGPPTYRADDEVICTSYYSRLVPLEHGEIHTSL |
| | | | INGRPSADDLSPKLLEFTSARYIRLRLQRIRTLNADL |
| | | | MTLSHREPKELDPIVTRRYYYSIKDISVGG |
| 35 | C | Mouse Lm α1 LEa-1 | ATGTGCATTTGCTACGGCCATGCCAGCAGCTGCCC |
| | | domain [DNA, 171 bp] | GTGGGATGAAGAAGCAAAGCAACTACAGTGTCAG |
| | | | TGTGAACACAATACGTGTGGCGAGAGCTGCGACA |
| | | | GGTGCTGTCCTGGCTACCATCAGCAGCCCTGGAGG |
| | | | CCCGGAACCATTTCCTCCGGCAACGAGTGTGAG |
| 36 | C | Mouse Lm α1 LEa-1 | MCICYGHASSCPWDEEAKQLQCQCEHNTCGESCDR |
| | | [required for LN | CCPGYHQQPWRPGTISSGNECE |
| | | folding; spacer domain] | |
| 37 | C | Human Lm α1 LEa-1 | ATGTGTATCTGCTATGGCCATGCTAGTAGCTGCCC |
| | | [DNA, 171 bp] | ATGGGATGAAACTACAAAGAAACTGCAGTGTCAA |
| | | | TGTGAGCATAATACTTGCGGGGAGAGCTGTAACA |
| | | | GGTGCTGTCCTGGGTACCATCAGCAGCCCTGGAGG |
| | | | CCGGGAACCGTGTCCTCCGGCAATACATGTGAA |
| 38 | C | Human Lm α1 LEa-1 | MCICYGHASSCPWDETTKKLQCQCEHNTCGESCNR |
| | | | CCPGYHQQPWRPGTVSSGNTCE |
| 39 | D | Mouse Lm α1 LEa-2 | GAATGCAACTGTCACAACAAAGCCAAAGATTGTT |
| | | domain [DNA, 210 bp] | ACTATGACAGCAGTGTTGCAAAGGAGAGGAGAAG |
| | | | CCTGAACACTGCCGGGCAGTACAGTGGAGGAGGG |
| | | | GTTTGTGTCAACTGCTCGCAGAATACCACAGGGAT |
| | | | CAACTGTGAAACCTGTATCGACCAGTATTACAGAC |
| | | | CTCACAAGGTATCTCCTTATGATGACCACCCTTGC |
| | | | CGT |
| 40 | D | Mouse Lm α1 LEa-2 | ECNCHNKAKDCYYDSSVAKERRSLNTAGQYSGGGV |
| | | [required for LN | CVNCSQNTTGINCETCIDQYYRPHKVSPYDDHPCR |
| | | folding; spacer domain] | |
| 41 | D | Human Lm α1 LEa-2 | GCATGTAATTGTCACAATAAAGCCAAAGACTGTTA |
| | | [DNA, 210 bp] | CTATGATGAAAGTGTTGCAAAGCAGAAGAAAAGT |
| | | | TTGAATACTGCTGGACAGTTCAGAGGAGGAGGGG |
| | | | TTTGCATAAATTGCTTGCAGAACACCATGGGAATC |
| | | | AACTGTGAAACCTGTATTGATGGATATTATAGACC |
| | | | ACACAAAGTGTCTCCTTATGAGGATGAGCCTTGCC |
| | | | GC |
| 42 | D | Human Lm α1 LEa-2 | ACNCHNKAKDCYYDESVAKQKKSLNTAGQFRGGG |
| | | | VCINCLQNTMGINCETCIDGYYRPHKVSPYEDEPCR |
| 43 | E | Mouse Lm α1 LEa-3 | CCCTGTAACTGTGACCCTGTGGGGTCTCTGAGTTC |
| | | domain [DNA, 171 bp] | TGTCTGTATCAAGGATGACCGCCATGCCGATTTAG |
| | | | CCAATGGAAAGTGGCCAGGTCAGTGTCCATGTAG |
| | | | GAAAGGTTATGCTGGAGATAAATGTGACCGCTGC |
| | | | CAGTTTGGCTACCGGGGTTTCCCAAATTGCATC |
| 44 | E | Mouse Lm α1 LEa-3 | PCNCDPVGSLSSVCIKDDRHADLANGKWPGQCPCR |
| | | [domain acting as | KGYAGDKCDRCQFGYRGFPNCI |
| | | spacer] | |
| 45 | E | Human Lm α1 LEa-3 | CCCTGTAATTGTGACCCTGTGGGGTCCCTCAGTTC |
| | | [DNA, 171 bp] | TGTCTGTATTAAGGATGACCTCCATTCTGACTTAC |
| | | | ACAATGGGAAGCAGCCAGGTCAGTGCCCATGTAA |
| | | | GGAAGGTTATACAGGAGAAAAATGTGATCGCTGC |
| | | | CAACTTGGCTATAAGGATTACCCGACCTGTGTC |
| 46 | E | Human Lm α1 LEa-3 | PCNCDPVGSLSSVCIKDDLHSDLHNGKQPGQCPCKE |
| | | | GYTGEKCDRCQLGYKDYPTCV |
| 47 | F | Mouse Lm α1 LEa-4 | CCCTGTGACTGCAGGACTGTCGGCAGCCTGAATGA |
| | | domain [DNA, 147 bp] | GGATCCATGCATAGAGCCGTGTCTTTGTAAGAAAA |
| | | | ATGTTGAGGGTAAGAACTGTGATCGCTGCAAGCC |
| | | | AGGATTCTACAACTTGAAGGAACGAAACCCCGAG |
| | | | GGCTGCTCC |
| 48 | F | Mouse Lm α1 LEa-4 | PCDCRTVGSLNEDPCIEPCLCKKNVEGKNCDRCKPG |
| | | [spacer domain] | FYNLKERNPEGCS |
| 49 | F | Human Lm α1 LEa-4 | TCCTGTGGGTGCAACCCAGTGGGCAGTGCCAGTG |
| | | [DNA, 147 bp] | ATGAGCCCTGCACAGGGCCCTGTGTTTGTAAGGAA |
| | | | AACGTTGAGGGGAAGGCCTGTGATCGCTGCAAGC |
| | | | CAGGATTCTATAACTTGAAGGAAAAAAACCCCCG |
| | | | GGGCTGCTCC |
| 50 | F | Human Lm α1 LEa-4 | SCGCNPVGSASDEPCTGPCVCKENVEGKACDRCKPG |
| | | | FYNLKEKNPRGCS |
| 51 | G | Mouse Lm α1 LF | GAGTGCTTCTGCTTCGGTGTCTCTGGTGTCTGT |
| | | domain LE-type | |
| | | fragment with 3 cys | |
| | | [DNA, 33 bp] | |
| 52 | G | Mouse Lm α1 LF | ECFCFGVSGVC |
| | | fragment (with 3 cys) | |
| | | [spacer segment] | |
| 53 | G | Human Lm α1 LF | GAGTGCTTCTGCTTTGGCGTTTCTGATGTCTGC |
| | | fragment (with 3 | |
| | | cys) [DNA, 33 bp] | |
| 54 | G | Human Lm α1 LF | CFCFGVSDVC |
| | | fragment (with 3 cys) | |
| 55 | H | Mouse Nidogen-1 G2 | CAGCAGACTTGTGCCAACAATAGACACCAGTGCT |
| | | domain [DNA, 843 bp] | CCGTGCATGCAGAGTGCAGAGACTATGCTACTGG |
| | | | CTTCTGCTGCAGGTGTGTGGCCAACTACACAGGCA |
| | | | ATGGCAGACAGTGCGTGGCAGAAGGCTCTCCACA |
| | | | ACGGGTCAATGGCAAGGTGAAGGGAAGGATCTTC |
| | | | GTGGGGAGCAGCCAGGTCCCCGTGGTGTTTGAGA |
| | | | ACACTGACCTGCACTCCTATGTGGTGATGAACCAC |
| | | | GGGCGCTCTTACACAGCCATCAGCACCATCCCTGA |
| | | | AACCGTCGGCTACTCTCTGCTCCCCCTGGCACCCA |
| | | | TTGGAGGCATCATCGGATGGATGTTTGCAGTGGAG |
| | | | CAGGATGGGTTCAAGAATGGGTTTAGCATCACTG |
| | | | GGGGCGAGTTTACCCGGCAAGCTGAGGTGACCTT |
| | | | CCTGGGGCACCCAGGCAAGCTGGTCCTGAAGCAG |
| | | | CAGTTCAGCGGTATTGATGAACATGGACACCTGAC |
| | | | CATCAGCACGGAGCTGGAGGGCCGCGTGCCGCAG |
| | | | ATCCCCTATGGAGCCTCGGTGCACATTGAGCCCTA |
| | | | CACCGAACTGTACCACTACTCCAGCTCAGTGATCA |
| | | | CTTCCTCCTCCACCCGGGAGTACACGGTGATGGAG |
| | | | CCTGATCAGGACGGCGCTGCACCCTCACACACCCA |
| | | | TATTTACCAGTGGCGTCAGACCATCACCTTCCAGG |
| | | | AGTGTGCCCACGATGACGCCAGGCCAGCCCTGCC |
| | | | CAGCACCCAGCAGCTCTCTGTGGACAGCGTGTTTG |
| | | | TCCTGTACAACAAGGAGGAGAGGATCTTGCGCTA |
| | | | TGCCCTCAGCAACTCCATCGGGCCTGTGAGGGATG |
| | | | GCTCCCCTGATGCC |
| 56 | H | Mouse Nidogen-1 G2 | QQTCANNRHQCSVHAECRDYATGFCCRCVANYTG |
| | | domain [direct | NGRQCVAEGSPQRVNGKVKGRIFVGSSQVPVVFENT |
| | | collagen-IV, perlecan | DLHSYVVMNHGRSYTAISTIPETVGYSLLPLAPIGGII |
| | | binding] | GWMFAVEQDGFKNGFSITGGEFTRQAEVTFLGHPG |
| | | | KLVLKQQFSGIDEHGHLTISTELEGRVPQIPYGASVHI |
| | | | EPYTELYHYSSSVITSSSTREYTVMEPDQDGAAPSHT |
| | | | HIYQWRQTITFQECAHDDARPALPSTQQLSVDSVFV |
| | | | LYNKEERILRYALSNSIGPVRDGSPDA |
| 57 | H | Human Nidogen-1 G2 | CGCCAGACGTGTGCTAACAACAGACACCAGTGCT |
| | | domain (direct | CGGTGCACGCAGAGTGCAGGGACTACGCCACGGG |
| | | collagen-IV, perlecan | CTTCTGCTGCAGCTGTGTCGCTGGCTATACGGGCA |
| | | binding)[DNA, 843 bp] | ATGGCAGGCAATGTGTTGCAGAAGGTTCCCCCCA |
| | | | GCGAGTCAATGGCAAGGTGAAAGGAAGGATCTTT |
| | | | GTGGGGAGCAGCCAGGTCCCCATTGTCTTTGAGAA |
| | | | CACTGACCTCCACTCTTACGTAGTAATGAACCACG |
| | | | GGCGCTCCTACACAGCCATCAGCACCATTCCCGAG |
| | | | ACCGTTGGATATTCTCTGCTTCCACTGGCCCCAGT |
| | | | TGGAGGCATCATTGGATGGATGTTTGCAGTGGAGC |
| | | | AGGACGGATTCAAGAATGGGTTCAGCATCACCGG |
| | | | GGGTGAGTTCACTCGCCAGGCTGAGGTGACCTTCG |
| | | | TGGGGCACCCGGGCAATCTGGTCATTAAGCAGCG |
| | | | GTTCAGCGGCATCGATGAGCATGGGCACCTGACC |
| | | | ATCGACACGGAGCTGGAGGGCCGCGTGCCGCAGA |
| | | | TTCCGTTCGGCTCCTCCGTGCACATTGAGCCCTAC |
| | | | ACGGAGCTGTACCACTACTCCACCTCAGTGATCAC |
| | | | TTCCTCCTCCACCCGGGAGTACACGGTGACTGAGC |
| | | | CCGAGCGAGATGGGGCATCTCCTTCACGCATCTAC |
| | | | ACTTACCAGTGGCGCCAGACCATCACCTTCCAGGA |
| | | | ATGCGTCCACGATGACTCCCGGCCAGCCCTGCCCA |
| | | | GCACCCAGCAGCTCTCGGTGGACAGCGTGTTCGTC |
| | | | CTGTACAACCAGGAGGAGAAGATCTTGCGCTATG |
| | | | CTCTCAGCAACTCCATTGGGCCTGTGAGGGAAGGC |
| | | | TCCCCTGATGCT |
| 58 | H | Human Nidogen-1 G2 | RQTCANNRHQCSVHAECRDYATGFCCSCVAGYTGN |
| | | domain (direct | GRQCVAEGSPQRVNGKVKGRIFVGSSQVPIVFENTD |
| | | collagen-IV, perlecan | LHSYVVMNHGRSYTAISTIPETVGYSLLPLAPVGGIIG |
| | | binding) | WMFAVEQDGFKNGFSITGGEFTRQAEVTFVGHPGN |
| | | | LVIKQRFSGIDEHGHLTIDTELEGRVPQIPFGSSVHIEP |
| | | | YTELYHYSTSVITSSSTREYTVTEPERDGASPSRIYTY |
| | | | QWRQTITFQECVHDDSRPALPSTQQLSVDSVFVLYN |
| | | | QEEKILRYALSNSIGPVREGSPDA |
| 59 | I | Mouse Nidogen-1 | CTTCAGAATCCATGCTACATTGGCACCCATGGGTG |
| | | EGF-like 2 domain | TGACAGCAATGCTGCCTGTCGCCCTGGCCCTGGAA |
| | | [126 bp] | CACAGTTCACCTGCGAATGCTCCATCGGCTTCCGA |
| | | | GGAGACGGGCAGACTTGCTAT |
| 60 | I | Mouse Nidogen-1 | LQNPCYIGTHGCDSNAACRPGPGTQFTCECSIGFRGD |
| | | EGF-like 2 [spacer] | GQTCY |
| 61 | I | Human Nidogen-1 | CTTCAGAATCCCTGCTACATCGGCACTCATGGGTG |
| | | EGF-like 2 domain | TGACACCAACGCGGCCTGTCGCCCTGGTCCCAGGA |
| | | [DNA, 126 bp] | CACAGTTCACCTGCGAGTGCTCCATCGGCTTCCGA |
| | | | GGAGACGGGCGAACCTGCTAT |
| 62 | I | Human Nidogen-1 | LQNPCYIGTHGCDTNAACRPGPRTQFTCECSIGFRGD |
| | | EGF-like 2 domain | GRTCY |
| 63 | J | Mouse Niogen-1 EGF- | GATATTGATGAGTGTTCAGAGCAGCCTTCCCGCTG |
| | | like 3 domain [126 bp]: | TGGGAACCATGCGGTCTGCAACAACCTCCCAGGA |
| | | | ACCTTCCGCTGCGAGTGTGTAGAGGGCTACCACTT |
| | | | CTCAGACAGGGGAACATGCGTG |
| 64 | J | Mouse Nidogen-1 | DIDECSEQPSRCGNHAVCNNLPGTFRCECVEGYHFS |
| | | EGF-like 3 | DRGTCV |
| 65 | J | Human Nidogen-1 | CTTCAGAATCCCTGCTACATCGGCACTCATGGGTG |
| | | EGF-like 3 domain | TGACACCAACGCGGCCTGTCGCCCTGGTCCCAGGA |
| | | [DNA, 126 bp] | CACAGTTCACCTGCGAGTGCTCCATCGGCTTCCGA |
| | | | GGAGACGGGCGAACCTGCTAT |
| 66 | J | Human Nidogen-1 | LQNPCYIGTHGCDTNAACRPGPRTQFTCECSIGFRGD |
| | | EGF-like 3 domain | GRTCY |
| 67 | K | Mouse Nidogen-1 | GCTGCCGAGGACCAACGT |
| | | spacer segment | |
| | | between EGF-3 and -4 | |
| | | [DNA, 18 bp] | |
| 68 | K | Mouse Nidogen-1 | AAEDQR |
| | | spacer segment | |
| | | between EGF-3 and -4 | |
| 69 | K | Human Nidogen-1 | GCTGTCGTGGACCAGCGC |
| | | spacer segment | |
| | | between EGF-3 and -4 | |
| | | [DNA, 18 bp] | |
| 70 | K | Human Nidogen-1 | AVVDQR |
| | | spacer segment | |
| | | between EGF-3 and -4 | |
| 71 | L | Mouse Nidogen-1 | CCCATCAACTACTGTGAAACTGGTCTCCACAACTG |
| | | EGF-like 4 domain | TGATATCCCCCAGCGAGCCCAGTGCATCTATATGG |
| | | [132 bp] | GTGGTTCCTCCTACACCTGCTCCTGTCTGCCTGGCT |
| | | | TCTCTGGGGATGGCAGAGCCTGCCGA |
| 72 | L | Mouse Nidogen-1 | PINYCETGLHNCDIPQRAQCIYMGGSSYTCSCLPGFS |
| | | EGF-like 4 | GDGRACR |
| 73 | L | Human Nidogen-1 | CCCATCAACTACTGTGAAACTGGCCTTCATAACTG |
| | | EGF-like 4 domain | CGACATACCCCAGCGGGCCCAGTGTATCTACACA |
| | | [DNA, 132 bp] | GGAGGCTCCTCCTACACCTGTTCCTGCTTGCCAGG |
| | | | CTTTTCTGGGGATGGCCAAGCCTGCCAA |
| 74 | L | Human Nidogen-1 | PINYCETGLHNCDIPQRAQCIYTGGSSYTCSCLPGFSG |
| | | EGF-like 4 domain | DGQACQ |
| 75 | M | Mouse Nidogen-1 | GACGTGGATGAATGCCAGCACAGCCGATGTCACC |
| | | EGF-like 5 domain | CCGATGCCTTCTGCTACAACACACCAGGCTCTTTC |
| | | [DNA, 141 bp] | ACATGTCAGTGCAAGCCTGGCTATCAGGGGGATG |
| | | | GCTTCCGATGCATGCCCGGAGAGGTGAGCAAAAC |
| | | | CCGG |
| 76 | M | Mouse Nidogen-1 | DVDECQHSRCHPDAFCYNTPGSFTCQCKPGYQGDG |
| | | EGF-like 5 [spacer] | FRCMPGEVSKTR |
| 77 | M | Human Nidogen-1 | GATGTAGATGAATGCCAGCCAAGCCGATGTCACC |
| | | EGF-like 5 domain | CTGACGCCTTCTGCTACAACACTCCAGGCTCTTTC |
| | | [DNA, 141 bp] | ACGTGCCAGTGCAAACCTGGTTATCAGGGAGACG |
| | | | GCTTCCGTTGCGTGCCCGGAGAGGTGGAGAAAAC |
| | | | CCGG |
| 78 | M | Human Nidogen-1 | DVDECQPSRCHPDAFCYNTPGSFTCQCKPGYQGDGF |
| | | EGF-like 5 domain | RCVPGEVEKTR |
| 79 | N | Mouse Nidogen-1 G3 | TGTCAACTGGAACGAGAGCACATCCTTGGAGCAG |
| | | TY (thyroglobulin-like) | CCGGCGGGGCAGATGCACAGCGGCCCACCCTGCA |
| | | domain [DNA, 282 bp] | GGGGATGTTTGTGCCTCAGTGTGATGAATATGGAC |
| | | | ACTATGTACCCACCCAGTGTCACCACAGCACTGGC |
| | | | TACTGCTGGTGTGTGGACCGAGATGGTCGGGAGCT |
| | | | GGAGGGTAGCCGTACCCCACCTGGGATGAGGCCC |
| | | | CCGTGTCTGAGTACAGTGGCTCCTCCTATTCACCA |
| | | | GGGACCAGTAGTACCTACAGCTGTCATCCCCCTGC |
| | | | CTCCA |
| 80 | N | Mouse Nidogen “G3” | CQLEREHILGAAGGADAQRPTLQGMFVPQCDEYGH |
| | | TY (thyroglobulin-like) | YVPTQCHHSTGYCWCVDRDGRELEGSRTPPGMRPP |
| | | domain | CLSTVAPPIHQGPVVPTAVIPLPP |
| 81 | N | Human Nidogen-1 G3 | TGCCAGCACGAGCGAGAACACATTCTCGGGGCAG |
| | | TY (thyroglobulin-like) | CGGGGGCGACAGACCCACAGCGACCCATTCCTCC |
| | | domain [DNA, 282 bp] | GGGGCTGTTCGTTCCTGAGTGCGATGCGCACGGGC |
| | | | ACTACGCGCCCACCCAGTGCCACGGCAGCACCGG |
| | | | CTACTGCTGGTGCGTGGATCGCGACGGCCGCGAG |
| | | | GTGGAGGGCACCAGGACCAGGCCCGGGATGACGC |
| | | | CCCCGTGTCTGAGTACAGTGGCTCCCCCGATTCAC |
| | | | CAAGGACCTGCGGTGCCTACCGCCGTGATCCCCTT |
| | | | GCCTCCT |
| 82 | N | Human Nidogen-1 G3 | CQHEREHILGAAGATDPQRPIPPGLFVPECDAHGHY |
| | | TY (thyroglobulin-like) | APTQCHGSTGYCWCVDRDGREVEGTRTRPGMTPPC |
| | | domain | LSTVAPPIHQGPAVPTAVIPLPP |
| 83 | O | Mouse Nidogen-1 G3 | GGGACACACTTACTCTTTGCTCAGACTGGAAAGAT |
| | | β-Prope11er domain | TGAACGCCTGCCCCTGGAAAGAAACACCATGAAG |
| | | [DNA, 744 bp] | AAGACAGAACGCAAGGCCTTTCTCCATATCCCTGC |
| | | | AAAAGTCATCATTGGACTGGCCTTTGACTGCGTGG |
| | | | ACAAGGTGGTTTACTGGACAGACATCAGCGAGCC |
| | | | TTCCATTGGGAGAGCCAGCCTCCACGGTGGAGAG |
| | | | CCAACCACCATCATTCGACAAGATCTTGGAAGCCC |
| | | | TGAAGGCATTGCCCTTGACCATCTTGGTCGAACCA |
| | | | TCTTCTGGACGGACTCTCAGTTGGATCGAATAGAA |
| | | | GTTGCAAAGATGGATGGCACCCAGCGCCGAGTGC |
| | | | TGTTTGACACGGGTTTGGTGAATCCCAGAGGCATT |
| | | | GTGACAGACCCCGTAAGAGGGAACCTTTATTGGA |
| | | | CAGATTGGAACAGAGATAATCCCAAAATTGAGAC |
| | | | TTCTCACATGGATGGCACCAACCGGAGGATTCTCG |
| | | | CACAGGACAACCTGGGCTTGCCCAATGGTCTGACC |
| | | | TTTGATGCATTCTCATCTCAGCTTTGCTGGGTGGAT |
| | | | GCAGGCACCCATAGGGCAGAATGCCTGAACCCAG |
| | | | CTCAGCCTGGCAGACGCAAAGTTCTCGAAGGGCT |
| | | | CCAGTATCCTTTCGCTGTGACTAGCTATGGGAAGA |
| | | | ATTTGTACTACACAGACTGGAAGACGAATTCAGTG |
| | | | ATTGCCATGGACCTTGCTATATCCAAAGAGATGGA |
| | | | TACCTTCCACCCACAC |
| 84 | O | Mouse Nidogen “G3” | GTHLLFAQTGKIERLPLERNTMKKTERKAFLHIPAKV |
| | | β-Prope11er [laminin- | ITGLAFDCVDKVVYWTDISEPSIGRASLHGGEPTTIIR |
| | | binding domain] | QDLGSPEGIALDHLGRTIFVVTDSQLDRIEVAKMDGT |
| | | | QRRVLFDTGLVNPRGIVTDPVRGNLYWTDWNRDNP |
| | | | KIETSHMDGTNRRILAQDNLGLPNGLTFDAFSSQLC |
| | | | WVDAGTHRAECLNPAQPGRRKVLEGLQYPFAVTSY |
| | | | GKNLYYTDWKTNSVIAMDLAISKEMDTFHPH |
| 85 | O | Human Nidogen-1 G3 | GGGACCCATTTACTCTTTGCCCAGACTGGGAAGAT |
| | | β-Prope11er domain | TGAGCGCCTGCCCCTGGAGGGAAATACCATGAGG |
| | | [DNA, 744 bp] | AAGACAGAAGCAAAGGCGTTCCTTCATGTCCCGG |
| | | | CTAAAGTCATCATTGGACTGGCCTTTGACTGCGTG |
| | | | GACAAGATGGTTTACTGGACGGACATCACTGAGC |
| | | | CTTCCATTGGGAGAGCTAGTCTACATGGTGGAGAG |
| | | | CCAACCACCATCATTAGACAAGATCTTGGAAGTCC |
| | | | AGAAGGTATCGCTGTTGATCACCTTGGCCGCAACA |
| | | | TCTTCTGGACAGACTCTAACCTGGATCGAATAGAA |
| | | | GTGGCGAAGCTGGACGGCACGCAGCGCCGGGTGC |
| | | | TCTTTGAGACTGACTTGGTGAATCCCAGAGGCATT |
| | | | GTAACGGATTCCGTGAGAGGGAACCTTTACTGGA |
| | | | CAGACTGGAACAGAGATAACCCCAAGATTGAAAC |
| | | | TTCCTACATGGACGGCACGAACCGGAGGATCCTTG |
| | | | TGCAGGATGACCTGGGCTTGCCCAATGGACTGACC |
| | | | TTCGATGCGTTCTCATCTCAGCTCTGCTGGGTGGA |
| | | | TGCAGGCACCAATCGGGCGGAATGCCTGAACCCC |
| | | | AGTCAGCCCAGCAGACGCAAGGCTCTCGAAGGGC |
| | | | TCCAGTATCCTTTTGCTGTGACGAGCTACGGGAAG |
| | | | AATCTGTATTTCACAGACTGGAAGATGAATTCCGT |
| | | | GGTTGCTCTCGATCTTGCAATTTCCAAGGAGACGG |
| | | | ATGCTTTCCAACCCCAC |
| 86 | O | Human Nidogen-1 G3 | GTHLLFAQTGKIERLPLEGNTMRKTEAKAFLHVPAK |
| | | β-Propeller domain | VIIGLAFDCVDKMVYWTDITEPSIGRASLHGGEPTTII |
| | | | RQDLGSPEGIAVDHLGRNIFVVTDSNLDRIEVAKLDG |
| | | | TQRRVLFETDLVNPRGIVTDSVRGNLYWTDWNRDN |
| | | | PKIETSYMDGTNRRILVQDDLGLPNGLTFDAFSSQLC |
| | | | WVDAGTNRAECLNPSQPSRRKALEGLQYPFAVTSY |
| | | | GKNLYFTDWKMNSVVALDLAISKETDAFQPH |
| 87 | P | Mouse Nidogen-1 G3 | AAGCAGACCCGGCTATATGGCATCACCATCGCCCT |
| | | EGF-like 6 domain | GTCCCAGTGTCCCCAAGGCCACAATTACTGCTCAG |
| | | [DNA, 171 bp] | TGAATAATGGTGGATGTACCCACCTCTGCTTGCCC |
| | | | ACTCCAGGGAGCAGGACCTGCCGATGTCCTGACA |
| | | | ACACCCTGGGAGTTGACTGCATTGAACGGAAA |
| 88 | P | Mouse Nidogen “G3” | KQTRLYGITIALSQCPQGHNYCSVNNGGCTHLCLPTP |
| | | EGF-like 6 [contacts | GSRTCRCPDNTLGVDCIERK* |
| | | laminin LE surface] | |
| 89 | P | Human Nidogen-1 G3 | AAGCAGACCCGGCTGTATGGCATCACCACGGCCC |
| | | EGF-like 6 domain | TGTCTCAGTGTCCGCAAGGCCATAACTACTGCTCA |
| | | [DNA, 162 bp] | GTGAACAATGGCGGCTGCACCCACCTATGCTTGGC |
| | | | CACCCCAGGGAGCAGGACCTGCCGTTGCCCTGAC |
| | | | AACACCTTGGGAGTTGACTGTATC |
| 90 | P | Human Nidogen-1 G3 | KQTRLYGITTALSQCPQGHNYCSVNNGGCTHLCLAT |
| | | EGF-like 6 domain | PGSRTCRCPDNTLGVDCI |
| 91 | Q | Mouse Laminin β1 | ATGGGGCTGCTCCAGGTGTTCGCCTTTGGTGTCCT |
| | | signal peptide [63 bp]: | AGCCCTATGGGGCACCCGAGTGTGCGCT |
| 92 | Q | Mouse Laminin β1 | MGLLQVFAFGVLALWGTRVCA |
| | | signal peptide | |
| 93 | Q | Human Laminin β1 | ATGGGGCTTCTCCAGTTGCTAGCTTTCAGTTTCTTA |
| | | signal [63 bp] | GCCCTGTGCAGAGCCCGAGTGCGCGCT |
| 94 | Q | Human Laminin β1 | MGLLQLLAFSFLALCRARVRA |
| | | signal peptide | |
| 95 | R | Mouse Laminin β1 LN | CAGGAACCGGAGTTCAGCTATGGCTGCGCAGAAG |
| | | domain [744 bp] | GCAGCTGCTACCCTGCCACTGGCGACCTTCTCATC |
| | | | GGCCGAGCGCAAAAGCTCTCCGTGACTTCGACAT |
| | | | GTGGACTGCACAAACCAGAGCCCTACTGTATTGTT |
| | | | AGCCACCTGCAGGAGGACAAGAAATGCTTCATAT |
| | | | GTGACTCCCGAGACCCTTATCACGAGACCCTCAAC |
| | | | CCCGACAGCCATCTCATTGAGAACGTGGTCACCAC |
| | | | ATTTGCTCCAAACCGCCTTAAGATCTGGTGGCAAT |
| | | | CGGAAAATGGTGTGGAGAACGTGACCATCCAACT |
| | | | GGACCTGGAAGCAGAATTCCATTTCACTCATCTCA |
| | | | TCATGACCTTCAAGACATTCCGCCCAGCCGCCATG |
| | | | CTGATCGAGCGGTCTTCTGACTTTGGGAAGACTTG |
| | | | GGGCGTGTACAGATACTTCGCCTACGACTGTGAGA |
| | | | GCTCGTTCCCAGGCATTTCAACTGGACCCATGAAG |
| | | | AAAGTGGATGACATCATCTGTGACTCTCGATATTC |
| | | | TGACATTGAGCCCTCGACAGAAGGAGAGGTAATA |
| | | | TTTCGTGCTTTAGATCCTGCTTTCAAAATTGAAGA |
| | | | CCCTTATAGTCCAAGGATACAGAATCTATTAAAAA |
| | | | TCACCAACTTGAGAATCAAGTTTGTGAAACTGCAC |
| | | | ACCTTGGGGGATAACCTTTTGGACTCCAGAATGGA |
| | | | AATCCGAGAGAAGTACTATTACGCTGTTTATGATA |
| | | | TGGTGGTTCGAGGG |
| 96 | R | Mouse Laminin β1 LN | QEPEFSYGCAEGSCYPATGDLLIGRAQKLSVTSTCGL |
| | | | HKPEPYCIVSHLQEDKKCFICDSRDPYHETLNPDSHLI |
| | | | ENVVTTFAPNRLKIVVWQSENGVENVTIQLDLEAEFH |
| | | | FTHLIMTFKTFRPAAMLIERSSDFGKTWGVYRYFAY |
| | | | DCESSFPGISTGPMKKVDDIICDSRYSDIEPSTEGEVIF |
| | | | RALDPAFKIEDPYSPRIQNLLKITNLRIKFVKLHTLGD |
| | | | NLLDSRMEIREKYYYAVYDMVVRG |
| 97 | R | Human Laminin β1 LN | CAGGAACCCGAGTTCAGCTACGGCTGCGCAGAAG |
| | | domain [DNA, 744 bp] | GCAGCTGCTATCCCGCCACGGGCGACCTTCTCATC |
| | | | GGCCGAGCACAGAAGCTTTCGGTGACCTCGACGT |
| | | | GCGGGCTGCACAAGCCCGAACCCTACTGTATCGTC |
| | | | AGCCACTTGCAGGAGGACAAAAAATGCTTCATAT |
| | | | GCAATTCCCAAGATCCTTATCATGAGACCCTGAAT |
| | | | CCTGACAGCCATCTCATTGAAAATGTGGTCACTAC |
| | | | ATTTGCTCCAAACCGCCTTAAGATTTGGTGGCAAT |
| | | | CTGAAAATGGTGTGGAAAATGTAACTATCCAACT |
| | | | GGATTTGGAAGCAGAATTCCATTTTACTCATCTCA |
| | | | TAATGACTTTCAAGACATTCCGTCCAGCTGCTATG |
| | | | CTGATAGAACGATCGTCCGACTTTGGGAAAACCTG |
| | | | GGGTGTGTATAGATACTTCGCCTATGACTGTGAGG |
| | | | CCTCGTTTCCAGGCATTTCAACTGGCCCCATGAAA |
| | | | AAAGTCGATGACATAATTTGTGATTCTCGATATTC |
| | | | TGACATTGAACCCTCAACTGAAGGAGAGGTGATA |
| | | | TTTCGTGCTTTAGATCCTGCTTTCAAAATAGAAGA |
| | | | TCCTTATAGCCCAAGGATACAGAATTTATTAAAAA |
| | | | TTACCAACTTGAGAATCAAGTTTGTGAAACTGCAT |
| | | | ACTTTGGGAGATAACCTTCTGGATTCCAGGATGGA |
| | | | AATCAGAGAAAAGTATTATTATGCAGTTTATGATA |
| | | | TGGTGGTTCGAGGA |
| 98 | R | Human Laminin β1 LN | QEPEFSYGCAEGSCYPATGDLLIGRAQKLSVTSTCGL |
| | | | HKPEPYCIVSHLQEDKKCFICNSQDPYHETLNPDSHL |
| | | | IENVVTTFAPNRLKIWWQSENGVENVTIQLDLEAEF |
| | | | HFTHLIMTFKTFRPAAMLIERSSDFGKTWGVYRYFA |
| | | | YDCEASFPGISTGPMKKVDDIICDSRYSDIEPSTEGEV |
| | | | IFRALDPAFKIEDPYSPRIQNLLKITNLRIKFVKLHTLG |
| | | | DNLLDSRMEIREKYYYAVYDMVVRG |
| 99 | S | Mouse Laminin β1 | AACTGCTTCTGCTATGGCCACGCCAGTGAATGCGC |
| | | LEa-1 domain [DNA, | CCCTGTGGATGGAGTCAATGAAGAAGTGGAAGGA |
| | | 192 bp] | ATGGTTCACGGGCACTGCATGTGCAGACACAACA |
| | | | CCAAAGGCCTGAACTGTGAGCTGTGCATGGATTTC |
| | | | TACCACGATTTGCCGTGGAGACCTGCTGAAGGCCG |
| | | | GAACAGCAACGCCTGCAAA |
| 100 | S | Mouse Laminin β1 | NCFCYGHASECAPVDGVNEEVEGMVHGHCMCRHN |
| | | LEa-1 | TKGLNCELCMDFYHDLPWRPAEGRNSNACK |
| 101 | S | Human Laminin β1 | AATTGCTTCTGCTATGGTCATGCCAGCGAATGTGC |
| | | LEa-1 [DNA, 192 bp] | CCCTGTGGATGGATTCAATGAAGAAGTGGAAGGA |
| | | | ATGGTTCACGGACACTGCATGTGCAGGCATAACA |
| | | | CCAAGGGCTTAAACTGTGAACTCTGCATGGATTTC |
| | | | TACCATGATTTACCTTGGAGACCTGCTGAAGGCCG |
| | | | AAACAGCAACGCCTGTAAA |
| 102 | S | Human Laminin β1 | NCFCYGHASECAPVDGFNEEVEGMVHGHCMCRHN |
| | | LEa-1 | TKGLNCELCMDFYHDLPWRPAEGRNSNACK |
| 103 | T | Mouse Laminin β1 | AAATGTAACTGCAATGAACATTCCAGCTCGTGTCA |
| | | LEa-2 domain [DNA, | CTTTGACATGGCAGTCTTCCTGGCTACTGGCAACG |
| | | 189 bp] | TCAGCGGGGGAGTGTGTGATAACTGTCAGCACAA |
| | | | CACCATGGGGCGCAACTGTGAACAGTGCAAACCG |
| | | | TTCTACTTCCAGCACCCTGAGAGGGACATCCGGGA |
| | | | CCCCAATCTCTGTGAA |
| 104 | T | Mouse Laminin β1 | KCNCNEHSSSCHFDMAVFLATGNVSGGVCDNCQHN |
| | | LEa-2 | TMGRNCEQCKPFYFQHPERDIRDPNLCE |
| 105 | T | Human Laminin β1 | AAATGTAACTGCAATGAACATTCCATCTCTTGTCA |
| | | LEa-2 [DNA, 189 bp] | CTTTGACATGGCTGTTTACCTGGCCACGGGGAACG |
| | | | TCAGCGGAGGCGTGTGTGATGACTGTCAGCACAA |
| | | | CACCATGGGGCGCAACTGTGAGCAGTGCAAGCCG |
| | | | TTTTACTACCAGCACCCAGAGAGGGACATCCGAG |
| | | | ATCCTAATTTCTGTGAA |
| 106 | T | Human Laminin β1 | KCNCNEHSISCHFDMAVYLATGNVSGGVCDDCQHN |
| | | LEa- | TMGRNCEQCKPFYYQHPERDIRDPNFCE |
| 107 | U | Mouse Laminin β1 | CCATGTACCTGTGACCCAGCTGGTTCTGAGAATGG |
| | | LEa-3 domain [DNA, | CGGGATCTGTGATGGGTACACTGATTTTTCTGTGG |
| | | 180 bp] | GTCTCATTGCTGGTCAGTGTCGGTGCAAATTGCAC |
| | | | GTGGAGGGAGAGCGCTGTGATGTTTGTAAAGAAG |
| | | | GCTTCTACGACTTAAGTGCTGAAGACCCGTATGGT |
| | | | TGTAAA |
| 108 | U | Mouse Laminin β1 | PCTCDPAGSENGGICDGYTDFSVGLIAGQCRCKLHV |
| | | LEa-3 | EGERCDVCKEGFYDLSAEDPYGCK |
| 109 | U | Human Laminin β1 | CGATGTACGTGTGACCCAGCTGGCTCTCAAAATGA |
| | | LEa-3 [DNA, 180 bp] | GGGAATTTGTGACAGCTATACTGATTTTTCTACTG |
| | | | GTCTCATTGCTGGCCAGTGTCGGTGTAAATTAAAT |
| | | | GTGGAAGGAGAACATTGTGATGTTTGCAAAGAAG |
| | | | GCTTCTATGATTTAAGCAGTGAAGATCCATTTGGT |
| | | | TGTAAA |
| 110 | U | Human Laminin β1 | RCTCDPAGSQNEGICDSYTDFSTGLIAGQCRCKLNVE |
| | | LEa-3 | GEHCDVCKEGFYDLSSEDPFGCK |
| 111 | V | Mouse Laminin β1 | TCATGTGCTTGCAATCCTCTGGGAACAATTCCTGG |
| | | LEa-4 domain [DNA, | TGGGAATCCTTGTGATTCTGAGACTGGCTACTGCT |
| | | 156 bp] | ACTGTAAGCGCCTGGTGACAGGACAGCGCTGTGA |
| | | | CCAGTGCCTGCCGCAGCACTGGGGTTTAAGCAATG |
| | | | ATTTGGATGGGTGTCGA |
| 112 | V | Mouse Laminin β1 | SCACNPLGTIPGGNPCDSETGYCYCKRLVTGQRCDQ |
| | | LEa-4 | CLPQHWGLSNDLDGCR |
| 113 | V | Human Laminin β1 | TCTTGTGCTTGCAATCCTCTGGGAACAATTCCTGG |
| | | LEa-4 [DNA, 156 bp] | AGGGAATCCTTGTGATTCCGAGACAGGTCACTGCT |
| | | | ACTGCAAGCGTCTGGTGACAGGACAGCATTGTGA |
| | | | CCAGTGCCTGCCAGAGCACTGGGGCTTAAGCAAT |
| | | | GATTTGGATGGATGTCGA |
| 114 | V | Human Laminin β1 | SCACNPLGTIPGGNPCDSETGHCYCKRLVTGQHCDQ |
| | | LEa-4 | CLPEHWGLSNDLDGCR |
| 115 | W | Mouse Laminin γ1 | ATGACGGGCGGCGGGCGGGCCGCGCTGGCCCTGC |
| | | signal peptide [DNA, | AGCCCCGGGGGCGGCTGTGGCCGCTGTTGGCTGTG |
| | | 99 bp] | CTGGCGGCTGTGGCGGGCTGTGTCCGGGCG |
| 116 | W | Mouse Laminin γ1 | MTGGGRAALALQPRGRLWPLLAVLAAVAGCVRA |
| | | signal peptide | |
| 117 | W | Human Laminin γl | ATGAGAGGGAGCCATCGGGCCGCGCCGGCCCTGC |
| | | signal peptide [DNA, | GGCCCCGGGGGCGGCTCTGGCCCGTGCTGGCCGT |
| | | 99 bp] | GCTGGCGGCGGCCGCCGCGGCGGGCTGTGCC |
| 118 | W | HUMAN Laminin | MRGSHRAAPALRPRGRLWPVLAVLAAAAAAGCA |
| | | γ1 signal peptide: | |
| 119 | X | Mouse Laminin γ1 LN | GCCATGGACTACAAGGACGACGATGACAAGGAGT |
| | | domain [DNA, 768 bp] | GCGCGGATGAGGGCGGGCGGCCGCAGCGCTGCAT |
| | | (note: E/GAG (2) in | GCCGGAGTTTGTTAATGCCGCCTTCAATGTGACCG |
| | | human γ1 vs D/GAC | TGGTGGCTACCAACACGTGTGGGACTCCGCCCGA |
| | | (1) D or E in mouse γI, | GGAGTACTGCGTGCAGACTGGGGTGACCGGAGTC |
| | | but E in crystal | ACTAAGTCCTGTCACCTGTGCGACGCCGGCCAGCA |
| | | structure of mouse LN- | GCACCTGCAACACGGGGCAGCCTTCCTGACCGACT |
| | | LEa) | ACAACAACCAGGCCGACACCACCTGGTGGCAAAG |
| | | | CCAGACTATGCTGGCCGGGGTGCAGTACCCCAACT |
| | | | CCATCAACCTCACGCTGCACCTGGGAAAGGCTTTT |
| | | | GACATCACTTACGTGCGCCTCAAGTTCCACACCAG |
| | | | CCGTCCAGAGAGCTTCGCCATCTATAAGCGCACTC |
| | | | GGGAAGACGGGCCCTGGATTCCTTATCAGTACTAC |
| | | | AGTGGGTCCTGTGAGAACACGTACTCAAAGGCTA |
| | | | ACCGTGGCTTCATCAGGACCGGAGGGGACGAGCA |
| | | | GCAGGCCTTGTGTACTGATGAATTCAGTGACATTT |
| | | | CCCCCCTCACCGGTGGCAACGTGGCCTTTTCAACC |
| | | | CTGGAAGGACGGCCGAGTGCCTACAACTTTGACA |
| | | | ACAGCCCTGTGCTCCAGGAATGGGTAACTGCCACT |
| | | | GACATCAGAGTGACGCTCAATCGCCTGAACACCTT |
| | | | TGGAGATGAAGTGTTTAACGAGCCCAAAGTTCTC |
| | | | AAGTCTTACTATTACGCAATCTCAGACTTTGCTGT |
| | | | GGGCGGC |
| 120 | X | Mouse Laminin γ1 LN | AMDECADEGGRPQRCMPEFVNAAFNVTVVATNTC |
| | | domain | GTPPEEYCVQTGVTGVTKSCHLCDAGQQHLQHGAA |
| | | | FLTDYNNQADTTWWQSQTMLAGVQYPNSINLTLHL |
| | | | GKAFDITYVRLKFHTSRPESFAIYKRTREDGPWIPYQ |
| | | | YYSGSCENTYSKANRGFIRTGGDEQQALCTDEFSDIS |
| | | | PLTGGNVAFSTLEGRPSAYNFDNSPVLQEWVTATDI |
| | | | RVTLNRLNTFGDEVFNEPKVLKSYYYAISDFAVGG |
| 121 | X | Human Laminin γ1 LN | CAGGCAGCCATGGACGAGTGCACGGACGAGGGCG |
| | | domain [DNA, 753 bp] | GGCGGCCGCAACGCTGCATGCCCGAGTTCGTCAA |
| | | | CGCCGCTTTCAACGTGACTGTGGTGGCCACCAACA |
| | | | CGTGTGGGACTCCGCCCGAGGAATACTGTGTGCA |
| | | | GACCGGGGTGACCGGGGTCACCAAGTCCTGTCAC |
| | | | CTGTGCGACGCCGGGCAGCCCCACCTGCAGCACG |
| | | | GGGCAGCCTTCCTGACCGACTACAACAACCAGGC |
| | | | CGACACCACCTGGTGGCAAAGCCAGACCATGCTG |
| | | | GCCGGGGTGCAGTACCCCAGCTCCATCAACCTCAC |
| | | | GCTGCACCTGGGAAAAGCTTTTGACATCACCTATG |
| | | | TGCGTCTCAAGTTCCACACCAGCCGCCCGGAGAGC |
| | | | TTTGCCATTTACAAGCGCACATGGGAAGACGGGC |
| | | | CCTGGATTCCTTACCAGTACTACAGTGGTTCCTGC |
| | | | GAGAACACCTACTCCAAGGCAAACCGCGGCTTCA |
| | | | TCAGGACAGGAGGGGACGAGCAGCAGGCCTTGTG |
| | | | TACTGATGAATTCAGTGACATTTCTCCCCTCACTG |
| | | | GGGGCAACGTGGCCTTTTCTACCCTGGAAGGAAG |
| | | | GCCCAGCGCCTATAACTTTGACAATAGCCCTGTGC |
| | | | TGCAGGAATGGGTAACTGCCACTGACATCAGTGT |
| | | | AACTCTTAATCGCCTGAACACTTTTGGAGATGAAG |
| | | | TGTTTAACGATCCCAAAGTTCTCAAGTCCTATTAT |
| | | | TATGCCATCTCTGATTTTGCTGTAGGTGGC |
| 122 | X | Human Laminin γ1 LN | QAAMDECTDEGGRPQRCMPEFVNAAFNVTVVATNT |
| | | domain | CGTPPEEYCVQTGVTGVTKSCHLCDAGQPHLQHGA |
| | | | AFLTDYNNQADTTWWQSQTMLAGVQYPSSINLTLH |
| | | | LGKAFDITYVRLKFHTSRPESFAIYKRTWEDGPWIPY |
| | | | QYYSGSCENTYSKANRGFIRTGGDEQQALCTDEFSDI |
| | | | SPLTGGNVAFSTLEGRPSAYNFDNSPVLQEWVTATD |
| | | | ISVTLNRLNTFGDEVFNDPKVLKSYYYAISDFAVGG |
| 123 | Y | Mouse Laminin γ1 | AGGTGTAAATGTAACGGACATGCCAGCGAGTGTG |
| | | LEa-1 domain [DNA, | TAAAGAACGAGTTTGACAAACTCATGTGCAACTG |
| | | 68 bp] | CAAACATAACACATACGGAGTTGACTGTGAAAAG |
| | | (note: TGC for cys | TGCCTGCCTTTCTTCAATGACCGGCCGTGGAGGAG |
| | | (Durkin, et al., | GGCGACTGCTGAGAGCGCCAGCGAGTGCCTT |
| | | Biochemistry 27 (14), | |
| | | 5198-5204 (1988); but | |
| | | earlier publications | |
| | | suggested TCC for | |
| | | serine (see, e.g., Sasaki | |
| | | and Yamada, J. Biol. | |
| | | Chem. 262 (35), 17111- | |
| | | 17117 (1987) | |
| 124 | Y | Mouse Laminin γ1 | RCKCNGHASECVKNEFDKLMCNCKHNTYGVDCEK |
| | | LEa-1 | CLPFFNDRPWRRATAESASECL |
| 125 | Y | Human Laminin γ1 | AGATGTAAATGTAATGGACACGCAAGCGAGTGTA |
| | | LEa-1 [DNA, 168 bp] | TGAAGAACGAATTTGATAAGCTGGTGTGTAATTGC |
| | | | AAACATAACACATATGGAGTAGACTGTGAAAAGT |
| | | | GTCTTCCTTTCTTCAATGACCGGCCGTGGAGGAGG |
| | | | GCAACTGCGGAAAGTGCCAGTGAATGCCTG |
| 126 | Y | Human Laminin γ1 | RCKCNGHASECMKNEFDKLVCNCKHNTYGVDCEK |
| | | LEa-1 | CLPFFNDRPWRRATAESASECL |
| 127 | Z | Mouse Laminin γ1 | CCTTGTGACTGCAATGGCCGATCCCAAGAGTGCTA |
| | | LEa-2 domain [DNA, | CTTTGATCCTGAACTATACCGTTCCACTGGACATG |
| | | 168 bp] | GTGGCCACTGTACCAACTGCCGGGATAACACAGA |
| | | | TGGTGCCAAGTGCGAGAGGTGCCGGGAGAATTTC |
| | | | TTCCGCCTGGGGAACACTGAAGCCTGCTCT |
| 128 | Z | Mouse Laminin γ1 | PCDCNGRSQECYFDPELYRSTGHGGHCTNCRDNTD |
| | | LEa-2 | GAKCERCRENFFRLGNTEACS |
| 129 | Z | Human Laminin γ1 | CCCTGTGATTGCAATGGTCGATCCCAGGAATGCTA |
| | | LEa-2 [DNA, 168 bp] | CTTCGACCCTGAACTCTATCGTTCCACTGGCCATG |
| | | | GGGGCCACTGTACCAACTGCCAGGATAACACAGA |
| | | | TGGCGCCCACTGTGAGAGGTGCCGAGAGAACTTC |
| | | | TTCCGCCTTGGCAACAATGAAGCCTGCTCT |
| 130 | Z | Human Laminin γ1 | PCDCNGRSQECYFDPELYRSTGHGGHCTNCQDNTD |
| | | LEa-2 | GAHCERCRENFFRLGNNEACS |
| 131 | a | Mouse Laminin γ1 | CCGTGCCACTGCAGCCCTGTTGGTTCTCTCAGCAC |
| | | LEa-3 domain [DNA, | ACAGTGTGACAGTTACGGCAGATGCAGCTGTAAG |
| | | 141 bp] | CCAGGAGTGATGGGTGACAAGTGTGACCGTTGTC |
| | | | AGCCTGGGTTCCATTCCCTCACTGAGGCAGGATGC |
| | | | AGG |
| 132 | a | Mouse Laminin γ1 | PCHCSPVGSLSTQCDSYGRCSCKPGVMGDKCDRCQP |
| | | LEa-3 | GFHSLTEAGCR |
| 133 | a | Human Laminin γ1 | TCATGCCACTGTAGTCCTGTGGGCTCTCTAAGCAC |
| | | LEa-3 [DNA, 141 bp] | ACAGTGTGATAGTTACGGCAGATGCAGCTGTAAG |
| | | | CCAGGAGTGATGGGGGACAAATGTGACCGTTGCC |
| | | | AGCCTGGATTCCATTCTCTCACTGAAGCAGGATGC |
| | | | AGG |
| 134 | a | Human Laminin γ1 | SCHCSPVGSLSTQCDSYGRCSCKPGVMGDKCDRCQP |
| | | LEa-3 | GFHSLTEAGCR |
| 135 | b | Mouse Laminin γ1 | CCATGCTCCTGCGATCTTCGGGGCAGCACAGACGA |
| | | LEa-4 [DNA, 150 bp] | GTGTAATGTTGAAACAGGAAGATGCGTTTGCAAA |
| | | | GACAATGTTGAAGGCTTCAACTGTGAGAGATGCA |
| | | | AACCTGGATTTTTTAATCTGGAGTCATCTAATCCT |
| | | | AAGGGCTGCACA |
| 136 | b | Mouse Laminin γ1 | PCSCDLRGSTDECNVETGRCVCKDNVEGFNCERCKP |
| | | LEa-4 | GFFNLESSNPKGCT |
| 137 | b | Human Laminin γ1 | CCATGCTCTTGTGATCCCTCTGGCAGCATAGATGA |
| | | LEa-4 [DNA, 150 bp] | ATGTAATGTTGAAACAGGAAGATGTGTTTGCAAA |
| | | | GACAATGTCGAAGGCTTCAATTGTGAAAGATGCA |
| | | | AACCTGGATTTTTTAATCTGGAATCATCTAATCCT |
| | | | CGGGGTTGCACA |
| 138 | b | Human Laminin γ1 | PCSCDPSGSIDECNVETGRCVCKDNVEGFNCERCKP |
| | | LEa-4 | GFFNLESSNPRGCT |
| 139 | c | Mouse agrin LG1 | CCCTCTGTGCCAGCTTTTAAGGGCCACTCCTTCTTG |
| | | domain [DNA, 531 bp] | GCCTTCCCCACCCTCCGAGCCTACCACACGCTGCG |
| | | | TCTGGCACTAGAATTCCGGGCGCTGGAGACAGAG |
| | | | GGACTGCTGCTCTACAATGGCAATGCACGTGGCA |
| | | | AAGATTTCCTGGCTCTGGCTCTGTTGGATGGTCAT |
| | | | GTACAGTTCAGGTTCGACACGGGCTCAGGGCCGG |
| | | | CGGTGCTAACAAGCTTAGTGCCAGTGGAACCGGG |
| | | | ACGGTGGCACCGCCTCGAGTTGTCACGGCATTGGC |
| | | | GGCAGGGCACACTTTCTGTGGATGGCGAGGCTCCT |
| | | | GTTGTAGGTGAAAGTCCGAGTGGCACTGATGGCCT |
| | | | CAACTTGGACACGAAGCTCTATGTGGGTGGTCTCC |
| | | | CAGAAGAACAAGTTGCCACGGTGCTTGATCGGAC |
| | | | CTCTGTGGGCATCGGCCTGAAAGGATGCATTCGTA |
| | | | TGTTGGACATCAACAACCAGCAGCTGGAGCTGAG |
| | | | CGATTGGCAGAGGGCTGTGGTTCAAAGCTCTGGTG |
| | | | TGGGGGAATGC |
| 140 | c | Mouse agrin LG1 | PSVPAFKGHSFLAFPTLRAYHTLRLALEFRALETEGL |
| | | domain | LLYNGNARGKDFLALALLDGHVQFRFDTGSGPAVL |
| | | | TSLVPVEPGRWHRLELSRHWRQGTLSVDGEAPVVG |
| | | | ESPSGTDGLNLDTKLYVGGLPEEQVATVLDRTSVGI |
| | | | GLKGCIRMLDINNQQLELSDWQRAVVQSSGVGEC |
| 141 | c | Human Agrin LG1 | GCCCCTGTGCCGGCCTTCGAGGGCCGCTCCTTCCT |
| | | [DNA, 531 bp] | GGCCTTCCCCACTCTCCGCGCCTACCACACGCTGC |
| | | | GCCTGGCACTGGAATTCCGGGCGCTGGAGCCTCA |
| | | | GGGGCTGCTGCTGTACAATGGCAACGCCCGGGGC |
| | | | AAGGACTTCCTGGCATTGGCGCTGCTAGATGGCCG |
| | | | CGTGCAGCTCAGGTTTGACACAGGTTCGGGGCCG |
| | | | GCGGTGCTGACCAGTGCCGTGCCGGTAGAGCCGG |
| | | | GCCAGTGGCACCGCCTGGAGCTGTCCCGGCACTG |
| | | | GCGCCGGGGCACCCTCTCGGTGGATGGTGAGACC |
| | | | CCTGTTCTGGGCGAGAGTCCCAGTGGCACCGACG |
| | | | GCCTCAACCTGGACACAGACCTCTTTGTGGGCGGC |
| | | | GTACCCGAGGACCAGGCTGCCGTGGCGCTGGAGC |
| | | | GGACCTTCGTGGGCGCCGGCCTGAGGGGGTGCAT |
| | | | CCGTTTGCTGGACGTCAACAACCAGCGCCTGGAGC |
| | | | TTGGCATTGGGCCGGGGGCTGCCACCCGAGGCTCT |
| | | | GGCGTGGGCGAGTGC |
| 142 | c | Human Agrin LG1 | APVPAFEGRSFLAFPTLRAYHTLRLALEFRALEPQGL |
| | | | LLYNGNARGKDFLALALLDGRVQLRFDTGSGPAVL |
| | | | TSAVPVEPGQWHRLELSRHWRRGTLSVDGETPVLG |
| | | | ESPSGTDGLNLDTDLFVGGVPEDQAAVALERTFVGA |
| | | | GLRGCIRLLDVNNQRLELGIGPGAATRGSGVGEC |
| 143 | d | Mouse agrin EGF-like | GGAGACCATCCCTGCTCACCTAACCCCTGCCATGG |
| | | domain 2 [DNA, 114 | CGGGGCCCTCTGCCAGGCCCTGGAGGCTGGCGTGT |
| | | bp] | TCCTCTGTCAGTGCCCACCTGGCCGCTTTGGCCCA |
| | | | ACTTGTGCA |
| 144 | d | Mouse agrin EGF-like | GDHPCSPNPCHGGALCQALEAGVFLCQCPPGRFGPT |
| | | domain 2 | CA |
| 145 | d | Human agrin EGF-like | GGGGACCACCCCTGCCTGCCCAACCCCTGCCATGG |
| | | domain 2 [DNA, 114 | CGGGGCCCCATGCCAGAACCTGGAGGCTGGAAGG |
| | | bp] | TTCCATTGCCAGTGCCCGCCCGGCCGCGTCGGACC |
| | | | AACCTGTGCC |
| 146 | d | Human Agrin EGF-like | GDHPCLPNPCHGGAPCQNLEAGRFHCQCPPGRVGPT |
| | | 2 | CA |
| 147 | e | Mouse agrin EGF-like | GATGAAAAGAACCCCTGCCAACCGAACCCCTGCC |
| | | domain 3 [DNA, 117 | ACGGGTCAGCCCCCTGCCATGTGCTTTCCAGGGGT |
| | | bp] | GGGGCCAAGTGTGCGTGCCCCCTGGGACGCAGTG |
| | | | GTTCCTTCTGTGAG |
| 148 | e | Mouse agrin EGF-like | DEKNPCQPNPCHGSAPCHVLSRGGAKCACPLGRSGS |
| | | domain 3 | FCE |
| 149 | e | Human Agrin EGF-like | GATGAGAAGAGCCCCTGCCAGCCCAACCCCTGCC |
| | | 3 [DNA, 117 bp] | ATGGGGCGGCGCCCTGCCGTGTGCTGCCCGAGGG |
| | | | TGGTGCTCAGTGCGAGTGCCCCCTGGGGCGTGAG |
| | | | GGCACCTTCTGCCAG |
| 150 | e | Human Agrin EGF-like | DEKSPCQPNPCHGAAPCRVLPEGGAQCECPLGREGT |
| | | 3 | FCQ |
| 151 | f | Mouse agrin LG | ACAGTCCTGGAGAATGCTGGCTCCCGG |
| | | Spacer-1 [DNA, 27 bp] | |
| 152 | f | Mouse agrin spacer | TVLENAGSR |
| | | domain-1 | |
| 153 | f | Human spacer [DNA, | ACAGCCTCGGGGCAGGACGGCTCTGGG |
| | | 27 bp] | |
| 154 | f | Human spacer | TASGQDGSG |
| 155 | g | Mouse agrin LG2 | CCCTTCCTGGCTGACTTTAATGGCTTCTCCTACCTG |
| | | domain [DNA, 537 bp] | GAACTGAAAGGCTTGCACACCTTCGAGAGAGACC |
| | | | TAGGGGAGAAGATGGCGCTGGAGATGGTGTTCTT |
| | | | GGCTCGTGGGCCCAGTGGCTTACTCCTCTACAATG |
| | | | GGCAGAAGACGGATGGCAAGGGGGACTTTGTATC |
| | | | CCTGGCCCTGCATAACCGGCACCTAGAGTTCCGCT |
| | | | ATGACCTTGGCAAGGGGGCTGCAATCATCAGGAG |
| | | | CAAAGAGCCCATAGCCCTGGGCACCTGGGTTAGG |
| | | | GTATTCCTGGAACGAAATGGCCGCAAGGGTGCCC |
| | | | TTCAAGTGGGTGATGGGCCCCGTGTGCTAGGGGA |
| | | | ATCTCCGGTCCCGCACACCATGCTCAACCTCAAGG |
| | | | AGCCCCTCTATGTGGGGGGAGCTCCTGACTTCAGC |
| | | | AAGCTGGCTCGGGGCGCTGCAGTGGCCTCCGGCTT |
| | | | TGATGGTGCCATCCAGCTGGTGTCTCTAAGAGGCC |
| | | | ATCAGCTGCTGACTCAGGAGCATGTGTTGCGGGCA |
| | | | GTAGATGTAGCGCCTTTT |
| 156 | g | Mouse agrin LG2 | PFLADFNGFSYLELKGLHTFERDLGEKMALEMVFLA |
| | | domain | RGPSGLLLYNGQKTDGKGDFVSLALHNRHLEFRYD |
| | | | LGKGAAIIRSKEPIALGTWVRVFLERNGRKGALQVG |
| | | | DGPRVLGESPVPHTMLNLKEPLYVGGAPDFSKLARG |
| | | | AAVASGFDGAIQLVSLRGHQLLTQEHVLRAVDVAPF |
| 157 | g | Human Agrin G2 | CCCTTCCTGGCTGACTTCAACGGCTTCTCCCACCT |
| | | [DNA, 537 bp] | GGAGCTGAGAGGCCTGCACACCTTTGCACGGGAC |
| | | | CTGGGGGAGAAGATGGCGCTGGAGGTCGTGTTCC |
| | | | TGGCACGAGGCCCCAGCGGCCTCCTGCTCTACAAC |
| | | | GGGCAGAAGACGGACGGCAAGGGGGACTTCGTGT |
| | | | CGCTGGCACTGCGGGACCGCCGCCTGGAGTTCCGC |
| | | | TACGACCTGGGCAAGGGGGCAGCGGTCATCAGGA |
| | | | GCAGGGAGCCAGTCACCCTGGGAGCCTGGACCAG |
| | | | GGTCTCACTGGAGCGAAACGGCCGCAAGGGTGCC |
| | | | CTGCGTGTGGGCGACGGCCCCCGTGTGTTGGGGG |
| | | | AGTCCCCGGTTCCGCACACCGTCCTCAACCTGAAG |
| | | | GAGCCGCTCTACGTAGGGGGCGCTCCCGACTTCAG |
| | | | CAAGCTGGCCCGTGCTGCTGCCGTGTCCTCTGGCT |
| | | | TCGACGGTGCCATCCAGCTGGTCTCCCTCGGAGGC |
| | | | CGCCAGCTGCTGACCCCGGAGCACGTGCTGCGGC |
| | | | AGGTGGACGTCACGTCCTTT |
| 158 | g | Human Agrin LG2 | PFLADFNGFSHLELRGLHTFARDLGEKMALEVVFLA |
| | | | RGPSGLLLYNGQKTDGKGDFVSLALRDRRLEFRYDL |
| | | | GKGAAVIRSREPVTLGAWTRVSLERNGRKGALRVG |
| | | | DGPRVLGESPVPHTVLNLKEPLYVGGAPDFSKLARA |
| | | | AAVSSGFDGAIQLVSLGGRQLLTPEHVLRQVDVTSF |
| 159 | h | Mouse agrin EGF-like | GCAGGCCACCCTTGTACCCAGGCCGTGGACAACC |
| | | domain 4 [DNA, 120 | CCTGCCTTAATGGGGGCTCCTGTATCCCGAGGGAA |
| | | bp] | GCCACTTATGAGTGCCTGTGTCCTGGGGGCTTCTC |
| | | | TGGGCTGCACTGCGAG |
| 160 | h | Mouse agrin EGF-like | AGHPCTQAVDNPCLNGGSCIPREATYECLCPGGFSG |
| | | domain 4 | LHCE |
| 161 | h | Human Agrin Egf-like | GCAGGTCACCCCTGCACCCGGGCCTCAGGCCACCC |
| | | 4 [DNA, 120 bp] | CTGCCTCAATGGGGCCTCCTGCGTCCCGAGGGAGG |
| | | | CTGCCTATGTGTGCCTGTGTCCCGGGGGATTCTCA |
| | | | GGACCGCACTGCGAG |
| 162 | h | Human Agrin EGF-like | AGHPCTRASGHPCLNGASCVPREAAYVCLCPGGFSG |
| | | 4 | PHCE |
| 163 | i | Mouse agrin LG | AAGGGGATAGTTGAGAAGTCAGTGGGGGAC |
| | | Spacer-2 [DNA, 30 bp] | |
| 164 | i | Mouse agrin LG | KGIVEKSVGD |
| | | Spacer-2 | |
| 165 | i | Human Spacer [30 bp] | AAGGGGCTGGTGGAGAAGTCAGCGGGGGAC |
| 166 | i | Human Spacer | KGLVEKSAGD |
| 167 | j | Mouse agrin LG3 | CTAGAAACACTGGCCTTTGATGGGCGGACCTACAT |
| | | domain [DNA, 537 bp] | CGAGTACCTCAATGCTGTGACTGAGAGTGAGAAA |
| | | | GCGCTGCAGAGCAACCACTTTGAGCTGAGCTTACG |
| | | | CACTGAGGCCACGCAGGGGCTGGTGCTGTGGATT |
| | | | GGAAAGGTTGGAGAACGTGCAGACTACATGGCTC |
| | | | TGGCCATTGTGGATGGGCACCTACAACTGAGCTAT |
| | | | GACCTAGGCTCCCAGCCAGTTGTGCTGCGCTCCAC |
| | | | TGTGAAGGTCAACACCAACCGCTGGCTTCGAGTCA |
| | | | GGGCTCACAGGGAGCACAGGGAAGGTTCCCTTCA |
| | | | GGTGGGCAATGAAGCCCCTGTGACTGGCTCTTCCC |
| | | | CGCTGGGTGCCACACAATTGGACACAGATGGAGC |
| | | | CCTGTGGCTTGGAGGCCTACAGAAGCTTCCTGTGG |
| | | | GGCAGGCTCTCCCCAAGGCCTATGGCACGGGTTTT |
| | | | GTGGGCTGTCTGCGGGACGTGGTAGTGGGCCATC |
| | | | GCCAGCTGCATCTGCTGGAGGACGCTGTCACCAA |
| | | | ACCAGAGCTAAGACCCTGC |
| 168 | j | Mouse agrin LG3 | LETLAFDGRTYIEYLNAVTESEKALQSNHFELSLRTE |
| | | domain | ATQGLVLWIGKVGERADYMALAIVDGHLQLSYDLG |
| | | | SQPVVLRSTVKVNTNRWLRVRAHREHREGSLQVGN |
| | | | EAPVTGSSPLGATQLDTDGALWLGGLQKLPVGQAL |
| | | | PKAYGTGFVGCLRDVVVGHRQLHLLEDAVTKPELR |
| | | | PC |
| 169 | j | Human Agrin LG3 | GTGGATACCTTGGCCTTTGACGGGCGGACCTTTGT |
| | | [DNA, 537 bp] | CGAGTACCTCAACGCTGTGACCGAGAGCGAGAAG |
| | | | GCACTGCAGAGCAACCACTTTGAACTGAGCCTGC |
| | | | GCACTGAGGCCACGCAGGGGCTGGTGCTCTGGAG |
| | | | TGGCAAGGCCACGGAGCGGGCAGACTATGTGGCA |
| | | | CTGGCCATTGTGGACGGGCACCTGCAACTGAGCTA |
| | | | CAACCTGGGCTCCCAGCCCGTGGTGCTGCGTTCCA |
| | | | CCGTGCCCGTCAACACCAACCGCTGGTTGCGGGTC |
| | | | GTGGCACATAGGGAGCAGAGGGAAGGTTCCCTGC |
| | | | AGGTGGGCAATGAGGCCCCTGTGACCGGCTCCTCC |
| | | | CCGCTGGGCGCCACGCAGCTGGACACTGATGGAG |
| | | | CCCTGTGGCTTGGGGGCCTGCCGGAGCTGCCCGTG |
| | | | GGCCCAGCACTGCCCAAGGCCTACGGCACAGGCT |
| | | | TTGTGGGCTGCTTGCGGGACGTGGTGGTGGGCCGG |
| | | | CACCCGCTGCACCTGCTGGAGGACGCCGTCACCA |
| | | | AGCCAGAGCTGCGGCCCTGC |
| 170 | j | Human Agrin LG3 | VDTLAFDGRTFVEYLNAVTESEKALQSNHFELSLRT |
| | | | EATQGLVLWSGKATERADYVALAIVDGHLQLSYNL |
| | | | GSQPVVLRSTVPVNTNRWLRVVAHREQREGSLQVG |
| | | | NEAPVTGSSPLGATQLDTDGALWLGGLPELPVGPAL |
| | | | PKAYGTGFVGCLRDVVVGRHPLHLLEDAVTKPELRP |
| | | | C |

### Example 9

### Simplification and Modification of LmαLNNdΔG2′ for Functional Enhancement

The current evaluated AAV-DJ constructs allow for inclusion of 3.1 kB DNA representing the open reading frame. Other constructs, existing or planned, whether using AAV-DJ, AAV8 or AAV9, can allow for larger inclusions. Basing allowed protein size on the AAV-DJ limit, it is noted that the nidogen G3 domain of LmαLNNdΔG2′ can be reduced in size to that of the propeller domain (˜270 residues, 810 bp), retaining laminin-binding as described in J. Takagi et al., 2003, Nature 424: 963-974. The reduction of 393 bp allows for domain rearrangement so that the G2 type IV collagen and perlecan-binding domain can be included. New arrangements allow for laminin polymerization to be coupled to collagen/perlecan binding. Examples are (a) αLNNdG2Propeller (3.08 kB) and (b) αLNNdG2Propeller-2 (3.02 kB). The domain composition for each of these is shown in Table 4 below using the letter domain coding provided in Table 2. The nucleotide and protein sequences for the domains used in the domain composition are provided in Table 3 and in the Sequence Listing. Another arrangement allows for laminin polymerization to be coupled to dystroglycan binding, an example of which is αLNNdPropellerAgrinLG (3.6 kB). The domain composition for αLNNdPropellerAgrinLG is shown in Table 4 below using the letter domain coding provided in Table 2. The nucleotide and protein sequences for the domains used in the domain composition are provided in Table 3 and in the Sequence Listing.

TABLE 4
Laminin Linker Proteins With Domain Composition By Letter Code⁷,⁸

| Linker Name | Domain Composition | Description | Size | Activity |
|---|---|---|---|---|
| αLNNdΔG2′ | ABCDEFGLMNOP | AAV | 3.02 | Binds to laminins with |
| | (or | expressed | | defective or absent α2 LN |
| | A′BCDEFGLMNOP) | linker protein | | domain near short arm |
| | | (Lmα1 and | | junction providing missing |
| | | nidogen-1 | | polymerization arm |
| | | chimera) to | | |
| | | ameliorate | | |
| | | LAMA2 MD | | |
| | | by enabling | | |
| | | polymerization | | |
| αLNNdΔ2EGF′ | ABCDHIJNOP | AAV | 3.48 | Alternative form that |
| | | expressed | | reduces LEa between LN |
| | | linker protein | | and G2 and EGF between |
| | | (Lm□1 and | | G2 and G3. |
| | | nidogen-1 | | |
| | | chimera) to | | |
| | | ameliorate | | |
| | | LAMA2 MD | | |
| | | by enabling | | |
| | | polymerization | | |
| | | and direct to | | |
| | | collagen- | | |
| | | IV/perlecan | | |
| | | binding | | |
| αLNNdΔLEa3,4 | ABCDGHIJKLMNOP | AAV | 3.79 | Alternative form that |
| | | expressed | | reduces LEa between LN |
| | | linker protein | | and G2. |
| | | (Lm□1 and | | |
| | | nidogen-1 | | |
| | | chimera) to | | |
| | | ameliorate | | |
| | | LAMA2 MD | | |
| | | by enabling | | |
| | | polymerization | | |
| | | and direct to | | |
| | | collagen- | | |
| | | IV/perlecan | | |
| | | binding | | |
| αLNNdΔEGF | ABCDHNOP | AAV | 3.23 | Alternative form that |
| | | expressed | | reduces LEa between LN |
| | | linker protein | | and G2 and removes all |
| | | (Lm□1 and | | EGF between G2 and G3 |
| | | nidogen-1 | | |
| | | chimera) to | | |
| | | ameliorate | | |
| | | LAMA2 MD | | |
| | | by enabling | | |
| | | polymerization | | |
| | | and direct to | | |
| | | collagen- | | |
| | | IV/perlecan | | |
| | | binding | | |
| αLNNdΔ2EGF′ | ABCHIJNOP | AAV | 3.27 | Alternative form that |
| minus LEa2 | | expressed | | reduces LEa between LN |
| | | linker protein | | and G2. |
| | | (Lm□1 and | | |
| | | nidogen-1 | | |
| | | chimera) to | | |
| | | ameliorate | | |
| | | LAMA2 MD | | |
| | | by enabling | | |
| | | polymerization | | |
| | | and direct to | | |
| | | collagen- | | |
| | | IV/perlecan | | |
| | | binding | | |
| αLNNdΔ2EGF′ | ABCHINOP | AAV | 3.14 | Alternative form that |
| minus LEa2, EGF3 | | expressed | | reduces LEa between LN |
| | | linker protein | | and G2 and EGF between |
| | | (Lm□1 and | | G2 and G3. |
| | | nidogen-1 | | |
| | | chimera) to | | |
| | | ameliorate | | |
| | | LAMA2 MD | | |
| | | by enabling | | |
| | | polymerization | | |
| | | and direct to | | |
| | | collagen- | | |
| | | IV/perlecan | | |
| | | binding | | |
| αLNNdΔG2 | ABCDLMNOP | AAV | 2.49 | Alternative form that |
| minus LEa3-4 | | expressed | | reduces LEa between LN |
| | | linker protein | | and EGF4 |
| | | (Lm□1 and | | |
| | | nidogen-1 | | |
| | | chimera) to | | |
| | | ameliorate | | |
| | | LAMA2 MD | | |
| | | by enabling | | |
| | | polymerization | | |
| αLNNdΔG2 | ABCDMNOP | AAV | 2.36 | Alternative form that |
| minus LEa3-4, EGF4 | | expressed | | reduces LEa and EGF |
| | | linker protein | | between LN and EGF5 |
| | | (Lm□1 and | | |
| | | nidogen-1 | | |
| | | chimera) to | | |
| | | ameliorate | | |
| | | LAMA2 MD | | |
| | | by enabling | | |
| | | polymerization | | |
| αLNNdG2Propeller | ABCDEH(J, K | AAV | 3.08 | Alternative form that |
| | or M)O | expressed | | reduces size of nidogen G3 |
| | | linker protein | | allowing insertion of G2 |
| | | to ameliorate | | domain |
| | | LAMA2 MD | | |
| | | by enabling | | |
| | | polymerization | | |
| | | and direct | | |
| | | collagen- | | |
| | | IV/perlecan | | |
| | | binding | | |
| αLNNdG2Propeller-2 | ABCDHIJO | AAV | 3.02 | Alternative form that |
| | | expressed | | reduces size of nidogen G3 |
| | | linker protein | | allowing insertion of G2 |
| | | to ameliorate | | domain |
| | | LAMA2 MD | | |
| | | by enabling | | |
| | | polymerization | | |
| | | and direct | | |
| | | collagen- | | |
| | | IV/perlecan | | |
| | | binding | | |
| αLNNdΔG2Propeller-3 | ABCDIJO | AAV | 2.18 | Alternative form that |
| | | expressed | | reduces size of nidogen |
| | | linker protein | | G3 and deletes G2. |
| | | to ameliorate | | |
| | | LAMA2 MD | | |
| | | by enabling | | |
| | | polymerization | | |
| | | without direct | | |
| | | collagen- | | |
| | | IV/perlecan | | |
| | | binding | | |
| αLNNdΔG2Propeller-4 | ABCDEFIJO | AAV | 2.53 | Alternative form that |
| | | expressed | | reduces size of nidogen |
| | | linker protein | | G3 and deletes G2. |
| | | to ameliorate | | |
| | | LAMA2 MD | | |
| | | by enabling | | |
| | | polymerization | | |
| | | without direct | | |
| | | collagen- | | |
| | | IV/perlecan | | |
| | | binding- | | |
| | | added spacers | | |
| αLNNdPropellerAgrinLG | ABCDEOPcdefg | linker protein | 3.60 | Alternative form for |
| | | to ameliorate | | polymerization and DG |
| | | LAMA2 MD | | binding (used with CKe8 |
| | | by enabling | | promoter) |
| | | polymerization | | |
| | | and | | |
| | | dystroglycan | | |
| | | binding | | |
| βLNNdΔG2′ | QRSTUVLMNOP | AAV | 2.99 | Binds to laminins with |
| | | expressed | | defective or absent 132 LN |
| | | linker protein | | domain near short arm |
| | | to ameliorate | | junction providing missing |
| | | Pierson | | polymerization arm |
| | | syndrome by | | |
| | | enabling | | |
| | | polymerization | | |
| βLNNdΔ2EGF′ | QRSTHIJNOP | AAV | 3.48 | Alternative form that |
| | | expressed | | reduces LEa between LN |
| | | linker protein | | and G2 and EGF between |
| | | (Lm□1 and | | G2 and G3 |
| | | nidogen-1 | | |
| | | chimera) to | | |
| | | ameliorate | | |
| | | Pierson' s | | |
| | | Syndrome by | | |
| | | enabling | | |
| | | polymerization | | |
| | | and direct to | | |
| | | collagen- | | |
| | | IV/perlecan | | |
| | | binding | | |
| βLNNdΔ2EGF′ | QRSHIJNOP | AAV | 3.27 | Alternative form that |
| minus LEa2 | | expressed | | reduces LE spacer domain |
| | | linker protein | | between LN and G2. |
| | | (Lm□1 and | | |
| | | nidogen-1 | | |
| | | chimera) to | | |
| | | ameliorate | | |
| | | Pierson's | | |
| | | Syndrome by | | |
| | | enabling | | |
| | | polymerization | | |
| | | and direct to | | |
| | | collagen- | | |
| | | IV/perlecan | | |
| βLNNdΔ2EGF′ | QRSHINOP | AAV | 3.14 | Alternative form that |
| minus LEa2, egf3 | | expressed | | reduces protein size by |
| | | linker protein | | removing spacer (LE, egf) |
| | | (Lm□1 and | | domains. |
| | | nidogen-1 | | |
| | | chimera) to | | |
| | | ameliorate | | |
| | | Pierson's | | |
| | | Syndrome by | | |
| | | enabling | | |
| | | polymerization | | |
| | | and direct to | | |
| | | collagen- | | |
| | | IV/perlecan | | |
| βLNNΔG2Propeller | QRSTUH(J, K | AAV | 3.08 | Alternative form that |
| | or M)O | expressed | | reduces size of nidogen G3 |
| | | linker protein | | domain complex and |
| | | to ameliorate | | deletes G2 domain. |
| | | Pierson | | |
| | | syndrome by | | |
| | | enabling | | |
| | | polymerization | | |
| | | and direct | | |
| | | collagen- | | |
| | | IV/perlecan | | |
| | | binding | | |
| γLNNdΔG2′ | WXYZabLMNOP | AAV | 3.01 | Binds to laminins with |
| | | expressed | | defective or absent γ1 or γ3 |
| | | linker protein | | LN domain near short arm |
| | | to ameliorate γ | | junction providing missing |
| | | subunit LN | | polymerization arm |
| | | deficiencies | | |
| γLNNdΔ2EGF′ | WXYZHIJNOP | | 3.50 | Alternative form that |
| | | | | reduces LEa between LN |
| | | | | and G2 and EGF between |
| | | | | G2 and G3 |
| γLNNdΔ2EGF′ | WXYHUNOP | | 3.29 | Alternative form that |
| minus LEa2 | | | | reduces LEa between LN |
| | | | | and G2. |
| γLNNdΔ2EGF′ minus | WXYHINOP | | 3.16 | Alternative form that |
| LEa2, egf3 | | | | reduces LEa between LN |
| | | | | and G2 and EGF between |
| | | | | G2 and G3 |
| γLNNΔG2Propeller | WXYZaH(J, K | AAV | 3.08 | Alternative form that |
| | or M)O | expressed | | reduces size of nidogen G3 |
| | | linker protein | | domain complex and |
| | | to ameliorate γ | | deletes G2 domain. |
| | | subunit LN | | |
| | | deficiencies by | | |
| | | enabling | | |
| | | polymerization | | |
| | | with direct | | |
| | | collagen- | | |
| | | IV/perlecan | | |
| | | binding | | |

⁷DNA open reading frame insert consists of the DNA domain segments ligated in the designated sequence
⁸LE and other EGF-like domains serve as inter-domain spacers and are considered interchangeable in constructs.

### Example 10

### Repair of Other Laminins With Polymerization Defects

Pierson syndrome is a congenital nephrotic syndrome with ocular abnormalities, leading to early end-stage renal disease, blindness and death. The causes are null, in-frame deleting or missense mutations in the LAMB2 gene that codes for the laminin β2 subunit. These mutations prevent subunit expression or alter the subunit properties. Several of the missense mutations are clustered in the β2 LN-domain (see Maatejas et al., 2010, Hum Mutat. 38: 992-1002 and K. K. McKee, M. Aleksandrova and P. D. Yurchenco, 2018, Matrix Biology 67: 32-46). The LN domain mediates polymerization of the laminin. The possible effects of these mutations are failure-to-fold the domain that can be low/non-secretors and failure to polymerize mutations. Two highly conserved mutations in Pierson syndrome (S80R and H147R) were evaluated after placing them into the β1 subunit (S68R and H135R). Both mutations greatly reduced polymerization, and it was found that βLNNd (β1 LN-LEa domains swapped for α1 LN-LEa in fusion with nidogen G3) was able to rescue recombinant laminin unable to polymerize because the laminin lacked the βLN domain (described in K. K. McKee, M. Aleksandrova and P. D. Yurchenco, 2018, Matrix Biology 67: 32-46.) Since βLNNd can repair the Pierson defects in vitro, it follows that the shorter βLNNdΔG2 can be used to treat the disease. Similarly, other diseases due to laminin LN mutations affecting polymerization are expected to be treatable by expression of related laminin linker proteins in which their corresponding LN-LEa segments have replaced the α1LN-LEa segment in the fusion protein. These proteins (βLNNdΔG2′, βLNNdG2Propeller, γLNNdΔG2′ and γLNNdG2Propeller) are described by domain composition in Tables 2 and 4 with sequences for the domains used in the domain composition provided in Table 3 and in the Sequence Listing.

### Example 11

### Direct Addition of Dystroglycan-binding Activity to αLNNdΔG2

Employment of the nidogen propeller domain instead of the full G3 domain complex creates room (in the context of allowed AAV insert size) for addition of a dystroglycan-binding domain. The protein is designated αLNNdΔG2PropellerAgrinLG. The domain composition is shown in Tables 2 and 4 with sequences for the domains used in the domain composition provided in Table 3 and in the Sequence Listing. The size increase here prevents use in the standard AAV-DJ virus and requires a virus that allows a larger insert such as one containing the smaller CK8e promoter.

### Example 12

### Delivery of Protein by Parenteral Injection

The LmαLNNdΔG2′ protein and any of its alternative forms can be injected parenterally (intraperitoneal, intra-vascular, intra-muscular routes) to deliver the protein to its intended tissue targets as an alternative to virally-delivered somatic gene therapy.

### Codon Optimization of Constructs

To optimize expression of the test constructs described herein not just as a means of reducing viral titers during the manufacturing process, but also to address safety concerns associated with large concentrations of the virus, the αLNNdΔG2′ transgene will be evaluated using a codon optimization process using freely available software (https://www.idtdna.com/CodonOpt). In addition, consensus Kozak sequences will be introduced into constructs as needed. Thus, any of the constructs or elements described herein may be codon optimized in this manner. Each of the modified constructs will be tested in parallel with the parental constructs in mice. Briefly, the constructs will be systemically administered through the temporal vein into mouse pups. The animals will then be euthanized either two or three weeks later and levels of protein from each of the constructs determined by Q-PCR and western blotting. Constructs delivering the most rapid and high levels of expression will be considered for eventual use in non-human primate studies and eventually in clinical trials for human patients.

### Example 13

### Reduction of Linker Protein Size by Removal of LE and EGF Spacer Domains

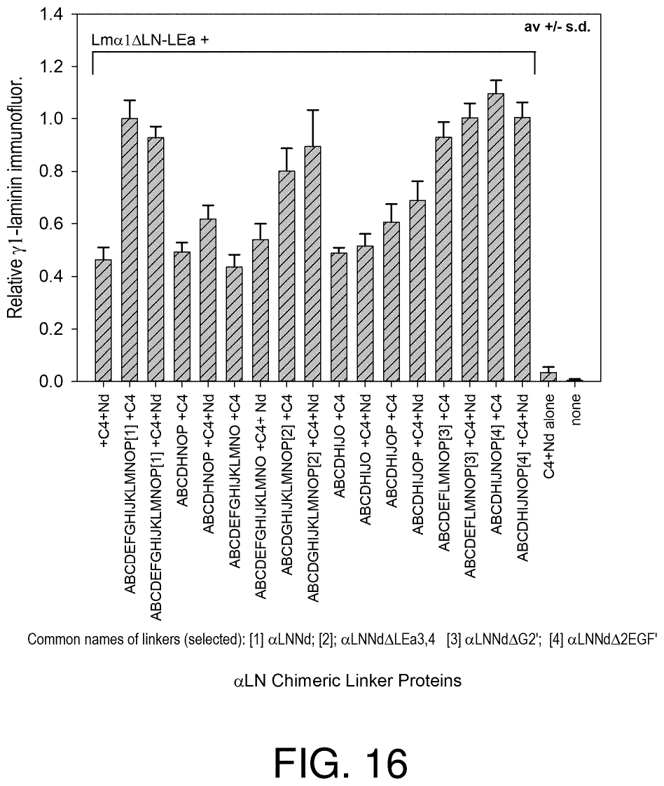

While the LEa1 domain is required for LN domain secretion, and removal of all EGF domains greatly reduces TyG3propellerEGF6 (“G3” domain complex) binding to laminins, intervening laminin type LE and nidogen EGF domains can be deleted to allow for inclusion of the nidogen G2 domain that binds to collagen-IV and perlecan. This is illustrated in FIG. 16 showing the results of linker protein mediation of laminin assembly on cultured myotubes.

Lawns of fused C2C12 myotubes were prepared. Non-polymerizing laminin (Lmα1ΔLN-LEa) was added to the medium of myotubes at 28 nM with 14 nM collagen-IV (C4) ±28 nM nidogen-1 (Nd) without or with 28 nM αLN linker protein. After incubation (37° C.), the cells were washed, fixed and incubated with γ1 laminin-specific antibody followed a secondary fluorescent-tagged antibody. After washing, images were recorded with a fluorescence microscope fitted with a digital camera. Images from 7 or more 10× fields were analyzed in ImageJ to determine the sum of fluorescence per field. Linker protein codes refer to Table 4. The linker proteins αLNNd (indicated as [1] in FIG. 16), αLNNdALEa3,4 (indicated as [2] in FIG. 16), αLNNdΔG2′ (indicated as [3] in FIG. 16) and αLNNdΔ2EGF′ (indicated as [4] in FIG. 16) all showed substantial and significant increased laminin on the myotube surfaces compared to the non-polymerizing laminin control. Several of the reduced-size linker proteins showed a two-fold increase in laminin assembly on myotubes relative to that obtained with non-polymerizing laminin.

### Example 14

### Potential for Endogenous Nidogen Competition for Linker Protein Binding to Laminins

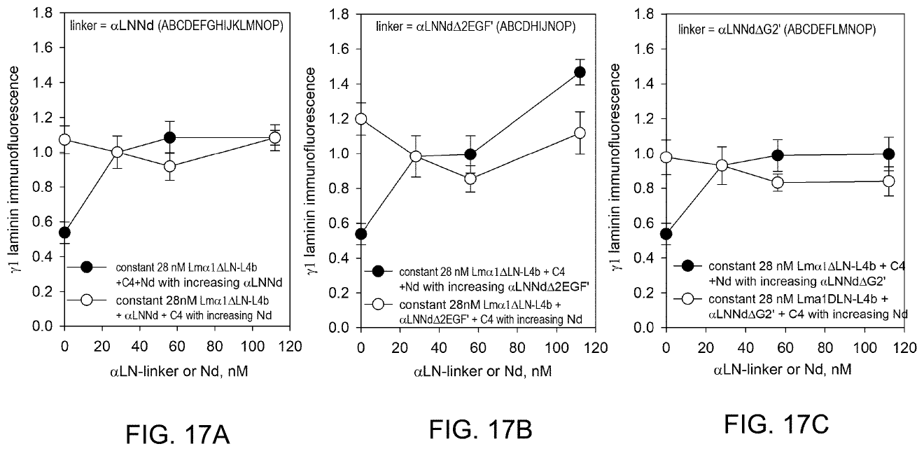

Endogenous nidogen-1 utilizes the same laminin binding site (Lmγ1LEb3) as the chimeric linker proteins. This allows for linker-nidogen competition during laminin-binding, potentially reducing the extent of laminin occupancy. However, it has been found that αLNNd binds sufficiently to non-polymerizing laminin in the dy2J mouse model to ameliorate the dystrophy and that competition favors the linker protein, as seen in an vitro competition experiment, likely because it gains polymerization activity (McKee et al. 2017. J. Clin. Invest. 127: 1075-1089). Two reduced-size linker proteins were compared for increasing laminin assembly on myotubes in the presence of equimolar and molar excess of nidogen-1 and found to be similar if not better. This is shown in FIG. 17A-C, which show nidogen competition for selected linker proteins and competition between three linker proteins and nidogen-1 on C2C12 myotubes.

The αLN linker proteins bind to the same locus in Lmγ1 as nidogen-1, leading to the prediction that the two proteins compete for laminin-binding. C2C12 myotubes were treated with different ratios of linkers αLNNd, αLNNdΔ2EGF′, αLNNdG2′ and nidogen-1 (Nd) co-incubated with 28 nM Lmα1ΔLn-L4b in the presence of 14 nM type IV collagen. FIG. 17A shows the results with linker αLNNd (ABCDEFGHIJKLMNOP). FIG. 17B shows the results with linker αLNNdΔ2EGF′ (ABCDHIJNOP). FIG. 17C shows the results with linker αLNNdG2′ (ABCDEFLMNOP). Increasing the nidogen/linker ratio with a non-polymerizing laminin decreased laminin accumulation on myotubes, whereas increasing the linker/nidogen ratio increased laminin accumulation. It appears that laminin polymerization gives an assembly advantage over nidogen alone, skewing accumulation in favor of the linker-modified laminins.

### Example 15

### Assembly of Reduced-Size Linker on Cells after Binding to Non-Polymerizing Lmα1ΔLN-L4b

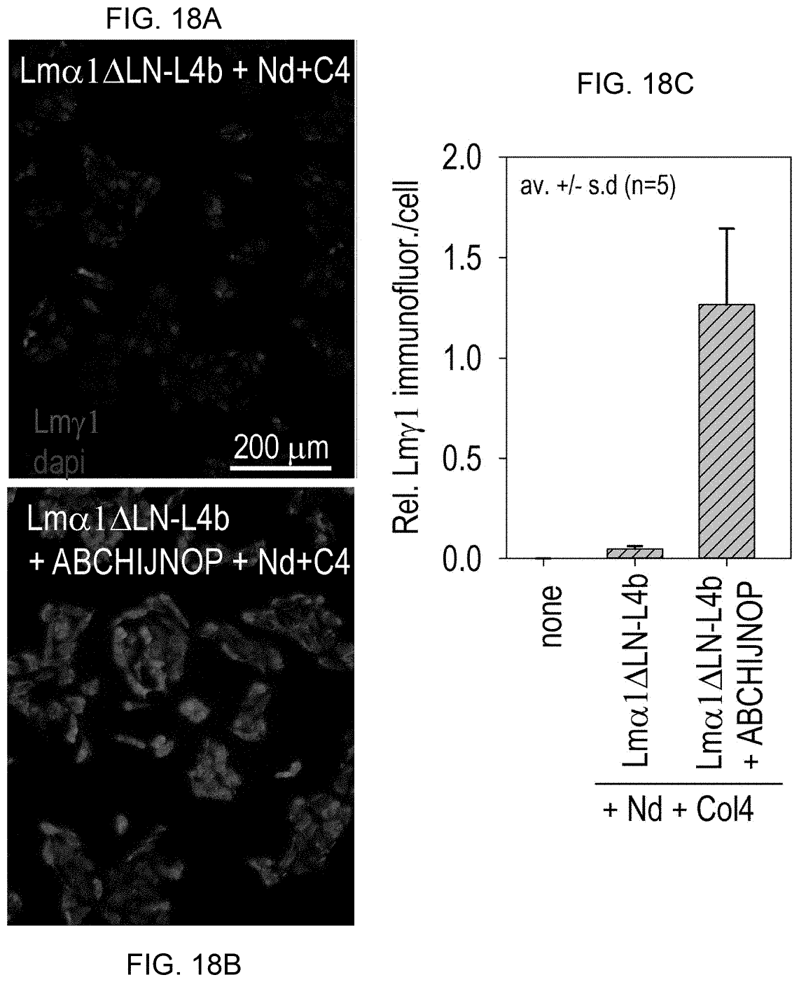

Conditioned medium containing laminin 111 lacking the α1 short arm polymerization domain (Lmα1ΔLN-L4b) was incubated with conditioned medium containing the linker protein αLNNdΔ2EGF′ minus LEa2, EGF3 (ABCHIJNOP) overnight. The medium containing the complex of proteins was added to a monolayer of cultured Schwann cells (a cell strain used to measure laminin/basement membrane assembly) containing collagen-IV and nidogen-1. After 1 hr, the cells were washed, fixed and immunostained for the laminin γ1 subunit and counterstained with dapi (nuclei). FIG. 18A shows the non-polymerizing laminin assembly on Schwann cell surfaces. FIG. 18B shows the increased accumulation of laminin with the gain of function of polymerizing protein. FIG. 18C provides a quantitative comparison of the non-polymerizing laminin assembly on Schwann cell surfaces of FIG. 18A with the increased accumulation of laminin with the gain of function of polymerizing protein of FIG. 18B. The linker, with a corresponding DNA open reading frame of 3.27 kB (compared to 4.15 kB for parental αLNNd), greatly increased the accumulation of laminin, i.e., substantially increased laminin assembly, on the cell surfaces.

### Example 16

### Linker Protein Insertions into AAV9 Constructs Using the CBh Promoter

The degree of laminin-binding linker protein repair depends upon the level of expression in muscle and peripheral nerve. The smaller the size of the cDNA coding for a linker protein, the greater the freedom in choosing promoters, enhancers and other stabilizing elements to achieve higher expression in tissues. On the other hand, recent advances in the development of promoters, enhancers and stabilizing elements have allowed for the reduction in their sizes. The AAV capsid limits the amount of total DNA to about 5 kB. Table 5 shows examples of alternative arrangements of these elements with linker proteins.

TABLE 5
αLN-linker Insertions in AAV-9 Constructs Using CBh General-Expression Promoter

| | | | | | | αLN- |
|---|---|---|---|---|---|---|
| | | | | | | LEa1- |
| | | | αLNNdΔG2′ | | | LEa2-G2- |
| | SIZE | | (“short | | | Egf2- |
| ITEM | (BP) | αLNNd | no G2”) | αLNNdΔEGF | αLNNdΔ2EGF′ | TyG3Egf6 |
| 5′ITR | 141 | 141 | 141 | 141 | 141 | 141 |
| intervening | 26 | 26 | 26 | 26 | 26 | 26 |
| kpn1 | 6 | 6 | 6 | 6 | 6 | 6 |
| CBh: CMV | 280 | 280 | 280 | 280 | 280 | 280 |
| enhancer/ | | | | | | |
| chick. b-actin | 266 | 266 | 266 | 266 | 266 | 266 |
| hybrid [CB] | | | | | | |
| promoter | | | | | | |
| SV40 enhancer = | 252 | 252 | 252 | 252 | 252 | 252 |
| “h” | | | | | | |
| Kozak | 6 | 6 | 6 | 6 | 6 | 6 |
| αLNNd with | 4152 | 4152 | | □ | | |
| endogenous | | | | | | |
| signal seq. | | | | | | |
| αLNNdΔG2 | | | 3009 | | | |
| αLNNdΔEGF | | | | 3230 | | |
| αLNNdΔ2EGF′ | | | | | 3480 | |
| αLNLea1LEa2G2Egf2G3 | | | | | | 3354 |
| αLNLEa1G2E2E3TyG3E6 | | | | | | |
| αLNLEa1G2E2TyG3E6 | | | | | | |
| WPRE | 598 | | 598 | 598 | | 598 |
| WPRE a/g (short) | | | | | | |
| poly(A) signal | 49 | 49 | 49 | 49 | 49 | 49 |
| SphI | 6 | 6 | 6 | 6 | 6 | 6 |
| intervening | 13 | 13 | 13 | 13 | 13 | 13 |
| 3′ ITR | 141 | 141 | 141 | 141 | 141 | 141 |
| TOTAL | | 5338 | 4793 | 5014 | 4666 | 5138 |
| comments | | too | fits well | near limit | fits | need to |
| concerning size: | | large | with | size | well | leave out |
| | | for | WPRE | | w/o | WPRE |
| | | most | | | WPRE | |
| | | promoters | | | | |

| | | | αLNNdΔ2E | | |
|---|---|---|---|---|---|
| | | | GF′ = LN- | | |
| | αLN- | αLN- | LEa1-LEa2- | | |
| | LEa1-G2- | LEa1-G2- | G2-EGF2- | | |
| | Egf2-Egf3- | Egf2- | EGF3- | TYPE | |
| ITEM | TyG3Egf6 | TyG3Egf6 | TyG3Egf6 | (element) | Comments |
| 5′ITR | 141 | 141 | 141 | ITR | AAV 5′ |
| | | | | | inverted |
| | | | | | terminal |
| | | | | | repeat |
| intervening | 26 | 26 | 26 | | |
| kpn1 | 6 | 6 | 6 | | intervening |
| | | | | | sequence |
| CBh: CMV | 280 | 280 | 280 | portion of | |
| enhancer/ | | | | the CMV | |
| | | | | immediate/ | |
| | | | | early | |
| | | | | enhancer | |
| chick. b-actin | 266 | 266 | 266 | Chicken-b- | Schwann/ |
| hybrid [CB] | | | | actin core | muscle gen′l |
| promoter | | | | promoter | promoter |
| SV40 enhancer = | 252 | 252 | 252 | | |
| “h” | | | | | |
| Kozak | 6 | 6 | 6 | | |
| αLNNd with | | | | ORF | linker |
| endogenous | | | | | protein, |
| signal seq. | | | | | full-length |
| αLNNdΔG2 | | | | ORF | small linker |
| | | | | | with G2 |
| αLNNdΔEGF | | | | | |
| αLNNdΔ2EGF′ | | | 3480 | | |
| αLNLea1LEa2G2Egf2G3 | | | | | |
| αLNLEalG2E2E3TyG3E6 | 3270 | | | | |
| aLNLEalG2E2TyG3E6 | | 3144 | | | |
| WPRE | 598 | 598 | | | stabilizes |
| | | | | | viral RNA |
| WPRE a/g (short) | | | 257 | | Schambach |
| | | | | | 2007 Mol |
| | | | | | Ther |
| | | | | | 15:1167 |
| poly(A) signal | 49 | 49 | 49 | | |
| SphI | 6 | 6 | 6 | misc. | |
| | | | | feature | |
| intervening | 13 | 13 | 13 | | |
| 3′ ITR | 141 | 141 | 141 | ITR | AAV 3′ |
| | | | | | inv. term. |
| | | | | | repeat |
| TOTAL | 5054 | 4928 | 4923 | | |
| comments | need to | fits with | fits with | | |
| concerning size: | leave out | WPRE | shorter | | |
| | WPRE | | WPRE and | | |
| | | | shorter polyA | | |

### REFERENCES

1. Donnelly, M. L. et al. (2001). The ‘cleavage’ activities of foot-and-mouth disease virus 2A site directed mutants and naturally occurring ‘2A-like’ sequences. J. Gen. Virol. 82, 1027-1041.
2. Foust, K. D., Nurre, E., Montgomery, C. L., Hernandez, A., Chan, C. M. and Kaspar, B. K. (2009). Intravascular AAV9 preferentially targets neonatal neurons and adult astrocytes. Nat. Biotech. 27, 59-65.
3. Grieger J C, Samulski R J (2005) Adeno-associated virus as a gene therapy vector: vector development, production and clinical applications. Adv Biochem Eng Biotechnol. 99, 119-145.
4. Grieger J C, Samulski R J (2012) Adeno-associated virus vectorology, manufacturing, and clinical applications. Methods Enzymol. 507, 229-254.
5. Kariya S, Re D B, Jacquier A, Nelson K, Przedborski S, Monani U R (2012) Mutant superoxide dismutase 1 (SOD1), a cause of amyotrophic lateral sclerosis, disrupts the recruitment of SMN, the spinal muscular atrophy protein to nuclear Cajal bodies. Hum Mol Genet. 21, 3421-3434.
6. Foust K D, Wang X, McGovern V L, Braun L, Bevan A K, Haidet A M, Le T T, Morales P R, Rich M M, Burghes A H, Kaspar B K (2010) Rescue of the spinal muscular atrophy phenotype in a mouse model by early postnatal delivery of SMN. Nat. Biotech. 28, 271-274.
7. Fleming J O, Ting J Y, Stohlman S A, Weiner L P (1983) Improvements in obtaining and characterizing mouse cerebrospinal fluid. Application to mouse hepatitis virus-induced encephalomyelitis. J Neuroimmunol. 4, 129-140.
8. Gao, G. P., and Sena-Esteves, M. (2012). Introducing Genes into Mammalian Cells: Viral Vectors. In Molecular Cloning, Vol 2: A Laboratory Manual (M. R. Green and J. Sambrook eds.) pp. 1209-1313. Cold Spring Harbor Laboratory Press, New York.
9. Rapti K, Louis-Jeune V, Kohlbrenner E, Ishikawa K, Ladage D, Zolotukhin S, Hajjar R J, Weber (2012) Neutralizing antibodies against AAV serotypes 1, 2, 6, and 9 in sea of commonly used animal models. Mol. Ther. 20, 73-83.
10. Goulder P J, Addo M M, Altfeld M A, Rosenberg E S, Tang Y, Govender U, Mngqundaniso N, Annamalai K, Vogel T U, Hammond M, Bunce M, Coovadia H M, Walker B D (2001) Rapid definition of five novel HLA-A*3002-restricted human immunodeficiency virus-specific cytotoxic T-lymphocyte epitopes by elispot and intracellular cytokine staining assays. J. Virol. 75, 1339-1347.
11. Aumailley, M., L. Bruckner-Tuderman, W. G. Carter, R. Deutzmann, D. Edgar, P. Ekblom, J. Engel, E. Engvall, E. Hohenester, J. C. Jones, H. K. Kleinman, M. P. Marinkovich, G. R. Martin, U. Mayer, G. Meneguzzi, J. H. Miner, K. Miyazaki, M.
12. Patarroyo, M. Paulsson, V. Quaranta, J. R. Sanes, T. Sasaki, K. Sekiguchi, L. M. Sorokin, J. F. Talts, K. Tryggvason, J. Uitto, I. Virtanen, K. von der Mark, U. M. Wewer, Y. Yamada, and P. D. Yurchenco, A simplified laminin nomenclature. Matrix Biol, 2005. 24(5): p. 326-32.
13. Jimenez-Mallebrera, C., S. C. Brown, C. A. Sewry, and F. Muntoni, Congenital muscular dystrophy: molecular and cellular aspects. Cell Mol Life Sci, 2005. 62(7-8): p. 809-23.
14. Sframeli, M., A. Sarkozy, M. Bertoli, G. Astrea, J. Hudson, M. Scoto, R. Mein, M. Yau, R. Phadke, L. Feng, C. Sewry, A. N. S. Fen, C. Longman, G. McCullagh, V. Straub, S. Robb, A. Manzur, K. Bushby, and F. Muntoni, Congenital muscular dystrophies in the UK population: Clinical and molecular spectrum of a large cohort diagnosed over a 12-year period. Neuromuscul Disord, 2017. 27(9): p. 793-803.
15. Allamand, V., Y. Sunada, M. A. Salih, V. Straub, C. O. Ozo, M. H. Al-Turaiki, M. Akbar, T. Kolo, H. Colognato, X. Zhang, L. M. Sorokin, P. D. Yurchenco, K. Tryggvason, and K. P. Campbell, Mild congenital muscular dystrophy in two patients with an internally deleted laminin alpha2-chain. Hum Mol Genet, 1997. 6(5): p. 747-52.
16. Gavassini, B. F., N. Carboni, J. E. Nielsen, E. R. Danielsen, C. Thomsen, K. Svenstrup, L. Bello, M. A. Maioli, G. Marrosu, A. F. Ticca, M. Mura, M. G. Marrosu, G. Soraru, C. Angelini, J. Vissing, and E. Pegoraro, Clinical and molecular characterization of limb girdle muscular dystrophy due to LAMA2 mutations. Muscle Nerve, 2011. 44(5): p. 703-9.
17. Bonnemann, C. G., C. H. Wang, S. Quijano-Roy, N. Deconinck, E. Bertini, A. Ferreiro, F. Muntoni, C. Sewry, C. Beroud, K. D. Mathews, S. A. Moore, J. Bellini, A. Rutkowski, and K. N. North, Diagnostic approach to the congenital muscular dystrophies. Neuromuscul Disord, 2014. 24(4): p. 289-311.
18. Chan, S. H., A. R. Foley, R. Phadke, A. A. Mathew, M. Pitt, C. Sewry, and F. Muntoni, Limb girdle muscular dystrophy due to LAMA2 mutations: diagnostic difficulties due to associated peripheral neuropathy. Neuromuscul Disord, 2014. 24(8): p. 677-83.
19. McKee, K. K., D. Harrison, S. Capizzi, and P. D. Yurchenco, Role of laminin terminal globular domains in basement membrane assembly. J Biol Chem, 2007. 282(29): p. 21437-47.
20. McKee, K. K., D. H. Yang, R. Patel, Z. L. Chen, S. Strickland, J. Takagi, K. Sekiguchi, and P. D. Yurchenco, Schwann Cell Myelination Requires Integration of Laminin Activities. J Cell Sci, 2012. 125(19): p. 4609-4619. PMC3500866
21. McKee, K. K., S. Capizzi, and P. D. Yurchenco, Scaffold-forming and adhesive contributions of synthetic laminin-binding proteins to basement membrane assembly. J Biol Chem, 2009. 284(13): p. 8984-8994. PMC2659255
22. Smirnov, S. P., P. Barzaghi, K. K. McKee, M. A. Ruegg, and P. D. Yurchenco, Conjugation of L G domains of agrins and perlecan to polymerizing laminin-2 promotes acetylcholine receptor clustering. J Biol Chem, 2005. 280(50): p. 41449-57.
23. Chang, C., H. L. Goel, H. Gao, B. Pursell, L. D. Shultz, D. L. Greiner, S. Ingerpuu, M. Patarroyo, S. Cao, E. Lim, J. Mao, K. K. McKee, P. D. Yurchenco, and A. M. Mercurio, Alaminin 511 matrix is regulated by TAZ and functions as the ligand for the alpha6Bbeta1 integrin to sustain breast cancer stem cells. Genes Dev, 2015. 29(1): p. 1-6. PMC4281560
24. Colombelli, C., M. Palmisano, Y. Eshed-Eisenbach, D. Zambroni, E. Pavoni, C. Ferri, S. Saccucci, S. Nicole, R. Soininen, K. K. McKee, P. D. Yurchenco, E. Peles, L. Wrabetz, and M. L. Feltri, Perlecan is recruited by dystroglycan to nodes of Ranvier and binds the clustering molecule gliomedin. J Cell Biol, 2015. 208(3): p. 313-29. PMC4315246
25. Yazlovitskaya, E. M., H. Y. Tseng, O. Viquez, T. Tu, G. Mernaugh, K. K. McKee, K. Riggins, V. Quaranta, A. Pathak, B. D. Carter, P. Yurchenco, A. Sonnenberg, R. T. Bottcher, A. Pozzi, and R. Zent, Integrin alpha3beta1 regulates kidney collecting duct development via TRAF6-dependent K63-linked polyubiquitination of Akt. Mol Biol Cell, 2015. 26(10): p. 1857-74. PMC4436831
26. Reuten, R., T. R. Patel, M. McDougall, N. Rama, D. Nikodemus, B. Gibert, J. G. Delcros, C. Prein, M. Meier, S. Metzger, Z. Zhou, J. Kaltenberg, K. K. McKee, T. Bald, T. Tuting, P. Zigrino, V. Djonov, W. Bloch, H. Clausen-Schaumann, E. Poschl, P. D. Yurchenco, M. Ehrbar, P. Mehlen, J. Stetefeld, and M. Koch, Structural decoding of netrin-4 reveals a regulatory function towards mature basement membranes. Nat Commun, 2016. 7: p. 13515. PMC514367
27. Choi, J., N. Yu, G. Baek, J. Bakes, D. Seo, H. J. Nam, S. H. Baek, C. Lim, Y. Lee, and B. Kaang, Optimization of AAV expression cassettes to improve packaging capacity and transgene expression in neurons. Molecular Brain, 2014. 7: p. 17-27
28. Gray S J, Foti S B, Schwartz J W, Bachaboina L, Taylor-Blake B, Coleman J, Ehlers M D, Zylka M J, McCown T J, Samulski R J C P. Optimizing promoters for recombinant adeno-associated virus mediated gene expression in the peripheral and central nervous system using self-complementary vectors. Hum Gene Ther. 2011; 22:1143-1153. [PubMed: 21476867] Grieger

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

Exemplary Recombinant Adeno-Associated Vectors, Compositions and Methods are Set Out in the Following Items:

Item 1. A recombinant adeno-associated vector (rAAV) comprising a nucleic acid sequence comprising a transgene encoding alphaLNNdDeltaG2short.
Item 2. The recombinant AAV of item 1, wherein the alphaLNNdDeltaG2short comprises SEQ ID NO: 1 or SEQ ID NO: 24.
Item 3. The recombinant AAV of item 1, wherein the AAV is AAV8, AAV-9 or AAV-DJ.
Item 4. The recombinant AAV of item 1, further comprising a CMV promoter.
Item 5. The recombinant AAV of item 4, wherein the CMV promoter comprises SEQ ID NO: 12.
Item 6. The recombinant AAV of item 1, wherein the recombinant vector further comprises inverted terminal repeats (ITRs).
Item 7. The recombinant AAV of item 6, wherein the inverted terminal repeat (ITR) is a 5′ ITR comprising SEQ ID NO: 11.
Item 8. The recombinant AAV of item 6, wherein the inverted terminal repeat (ITR) is a 3′ ITR comprising SEQ ID NO: 16.
Item 9. A recombinant adeno-associated vector (rAAV) comprising a transgene encoding a variant alphaLNNd wherein the variant alphaLNND comprises a nucleic acid sequence comprising SEQ ID NO: 171, SEQ ID NO: 173, SEQ ID NO; 175, SEQ ID NO; 179, SEQ ID NO: 1 or SEQ ID NO: 24. [
Item 10. A recombinant adeno-associated vector (rAAV) comprising a nucleic acid sequence comprising a transgene encoding alphaLNNdDeltaG2Propeller, wherein the nucleic acid sequence comprises either: (a) SEQ ID NOS: 25, 29, 31, 33, 35, 41, 45 and 55; (b) SEQ ID NOS: 25, 29, 31, 33, 35, 41, 47 and 55; or (c) SEQ ID NOS: 25, 29, 31, 33, 35, 41, 51 and 55.
Item 11. A recombinant adeno-associated vector (rAAV) comprising a nucleic acid sequence comprising a transgene encoding alphaLNNdDeltaG2Propeller-2, wherein the nucleic acid sequence comprises SEQ ID NOS: 25, 29, 31, 33, 41, 43, 45 and 55.
Item 12. A recombinant adeno-associated vector (rAAV) comprising a nucleic acid sequence comprising a transgene encoding betaLNNdDeltaG2short, wherein the nucleic acid sequence comprises SEQ ID NOS: 59, 63, 67, 71, 75, 79, 49, 51, 53, 55 and 57.
Item 13. A recombinant adeno-associated vector (rAAV) comprising a nucleic acid sequence comprising a transgene encoding gammaLNNdDeltaG2short, wherein the nucleic acid sequence comprises SEQ ID NOS: 83, 87, 91, 95, 99, 103, 49, 51, 53, 55 and 57.
Item 14. A pharmaceutical composition comprising the recombinant AAV of items 1, 2, 9, 10, 11, 12 or 13 and a pharmaceutical carrier.
Item 15. A kit comprising a container housing comprising the composition of item 14.
Item 16. A method of restoring laminin polymerization expression and basement membrane assembly in a subject, comprising administering to the subject an effective amount of the recombinant AAV vector of items 1, 2, 9, 10, 11, 12 or 13.
Item 17. A method of treating laminin α-2 deficiency syndrome in a subject in need thereof, wherein the method comprises administering to the subject an effective amount of the recombinant AAV vector of item 1 or 9.
Item 18. A method of alleviating in a subject at least one of the symptoms associated with laminin deficiencies selected from the group consisting of laminin-deficient muscular dystrophies and laminin α2-deficient muscular dystrophy, wherein the method comprises administering to the subject an effective amount of the recombinant AAV vector of item 1 or 9.
Item 19. A method of alleviating in a subject at least one of the symptoms associated with laminin α2-deficiencies selected from the group consisting of muscle degeneration, regeneration, chronic inflammation, fibrosis, white matter brain anomalies, reduced peripheral nerve conduction, seizures, moderate mental retardation, and respiratory failure, wherein the method comprises administering to the subject an effective amount of the recombinant AAV vector of item 1 or 9.
Item 20. The method of item 17, 18 or 19, wherein the alphaLNNdDeltaG2short comprises SEQ ID NO: 1 or SEQ ID NO: 24.
Item 21. The method of item 17, 18 or 19, wherein the AAV is AAV8, AAV-9 or AAV-DJ.
Item 22. The method of item 17, 18 or 19, wherein the recombinant AAV further comprises a Item CMV promoter.
Item 23. The method of item 22, wherein the wherein the CMV promoter comprises SEQ ID NO: 12.
Item 24. The method of item 17, 18 or 19, wherein the recombinant vector further comprises inverted terminal repeats (ITRs).
Item 25. The method of item 24, wherein the inverted terminal repeat (ITR) is a 5′ ITR comprising SEQ ID NO: 11.
Item 26. The method of item 24, wherein the inverted terminal repeat (ITR) is a 3′ ITR comprising SEQ ID NO: 16.
Item 27. The method of item 17, 18 or 19, wherein the recombinant AAV is comprised within a pharmaceutical composition further comprising a pharmaceutical carrier.

1. A recombinant adeno-associated vector (rAAV) comprising a transgene encoding a variant alphaLNNd wherein the variant alphaLNND comprises a nucleic acid sequence comprising SEQ ID NO: 171, SEQ ID NO: 173, SEQ ID NO; 175, or SEQ ID NO: 179.

2. The recombinant AAV of claim 1, wherein the AAV is AAV8, AAV-9 or AAV-DJ.

3. The recombinant AAV of claim 1, further comprising a CMV promoter.

4. The recombinant AAV of claim 3, wherein the CMV promoter comprises SEQ ID NO: 12.

5. The recombinant AAV of claim 1, wherein the recombinant vector further comprises inverted terminal repeats (ITRs).

6. The recombinant AAV of claim 5, wherein the inverted terminal repeat (ITR) is a 5′ ITR comprising SEQ ID NO: 11.

7. The recombinant AAV of claim 5, wherein the inverted terminal repeat (ITR) is a 3′ ITR comprising SEQ ID NO: 16.

8. A pharmaceutical composition comprising the recombinant AAV of claim 1 and a pharmaceutical carrier.

9. A method of restoring laminin polymerization expression and basement membrane assembly in a subject, comprising administering to the subject an effective amount of the recombinant AAV vector of claim 1.

10. A method of treating laminin α-2 deficiency syndrome in a subject in need thereof, wherein the method comprises administering to the subject an effective amount of the recombinant AAV vector of claim 1.

11. A method of alleviating in a subject at least one of the symptoms associated with laminin deficiencies selected from the group consisting of laminin-deficient muscular dystrophies and laminin α2-deficient muscular dystrophy, wherein the method comprises administering to the subject an effective amount of the recombinant AAV vector of claim 1.

12. A method of alleviating in a subject at least one of the symptoms associated with laminin α2-deficiencies selected from the group consisting of muscle degeneration, regeneration, chronic inflammation, fibrosis, white matter brain anomalies, reduced peripheral nerve conduction, seizures, moderate mental retardation, and respiratory failure, wherein the method comprises administering to the subject an effective amount of the recombinant AAV vector of claim 1.

13. The method of claim 10, wherein the AAV is AAV8, AAV-9 or AAV-DJ.

14. The method of claim 10, wherein the recombinant AAV further comprises a CMV promoter.

15. The method of claim 10, wherein the recombinant vector further comprises inverted terminal repeats (ITRs).

16. A recombinant adeno-associated vector (rAAV) comprising a transgene encoding a variant alphaLNNd wherein the variant alphaLNND comprises a nucleic acid sequence comprising SEQ ID NO: 1 or SEQ ID NO: 24, and wherein the AAV is AAV9.


SEQUENCE LISTING (180 sequences)

SEQ ID NO: 1 - DNA, 3009 bp, Artificial Sequence
  alphaLNNdDeltaG2 open reading frame no tag used in the AAV construct
atgagggcctggatcttctttctcctttgcctggccgggagggctctggcacagcagagaggcttgttccctgccattctcaacctggccaccaatgcccacatcagcgccaatgctacctgtggagagaaggggcctgagatgttctgcaaactcgtggagcacgtgccgggccggcctgttcgacacgcccaatgccgggtctgtgacggtaacagtacgaatcctagagagcgccatccgatatcacacgcaatcgatggcaccaacaactggtggcagagccccagtattcagaatgggagagagtatcactgggtcactgtcaccctggacttacggcaggtctttcaagttgcatacatcatcattaaagctgccaatgcccctcggcctggaaactggattttggagcgctccgtggatggcgtcaagttcaaaccctggcagtactatgccgtcagcgatacagagtgtttgacccgctacaaaataactccacggcggggacctcccacttacagagcagacaacgaagtcatctgcacctcgtattattcaaagctggtgccacttgaacatggagagattcacacatcactcatcaatggcagacccagcgctgacgacccctcaccccagttgctggaattcacctcagcacggtacattcgccttcgtcttcagcgcatcagaacactcaacgcagacctcatgacccttagccatcgggacctcagagaccttgaccccattgtcacaagacgttattactattcgataaaagacatttccgttggaggcatgtgcatttgctacggccatgccagcagctgcccgtgggatgaagaagcaaagcaactacagtgtcagtgtgaacacaatacgtgtggcgagagctgcgacaggtgctgtcctggctaccatcagcagccctggaggcccggaaccatttcctccggcaacgagtgtgaggaatgcaactgtcacaacaaagccaaagattgttactatgacagcagtgttgcaaaggagaggagaagcctgaacactgccgggcagtacagtggaggaggggtttgtgtcaactgctcgcagaataccacagggatcaactgtgaaacctgtatcgaccagtattacagacctcacaaggtatctccttatgatgaccacccttgccgtccctgtaactgtgaccctgtggggtctctgagttctgtctgtatcaaggatgaccgccatgccgatttagccaatggaaagtggccaggtcagtgtccatgtaggaaaggttatgctggagataaatgtgaccgctgccagtttggctaccggggtttcccaaattgcatcccctgtgactgcaggactgtcggcagcctgaatgaggatccatgcatagagccgtgtctttgtaagaaaaatgttgagggtaagaactgtgatcgctgcaagccaggattctacaacttgaaggaacgaaaccccgagggctgctccgagtgcttctgcttcggtgtctctggtgtctgtcccatcaactactgtgaaactggtctccacaactgtgatatcccccagcgagcccagtgcatctatatgggtggttcctcctacacctgctcctgtctgcctggcttctctggggatggcagagcctgccgagacgtggatgaatgccagcacagccgatgtcaccccgatgccttctgctacaacacaccaggctctttcacatgtcagtgcaagcctggctatcagggggatggcttccgatgcatgcccggagaggtgagcaaaacccggtgtcaactggaacgagagcacatccttggagcagccggcggggcagatgcacagcggcccaccctgcaggggatgtttgtgcctcagtgtgatgaatatggacactatgtacccacccagtgtcaccacagcactggctactgctggtgtgtggaccgagatggtcgggagctggagggtagccgtaccccacctgggatgaggcccccgtgtctgagtacagtggctcctcctattcaccagggaccagtagtacctacagctgtcatccccctgcctccagggacacacttactctttgctcagactggaaagattgaacgcctgcccctggaaagaaacaccatgaagaagacagaacgcaaggcctttctccatatccctgcaaaagtcatcattggactggcctttgactgcgtggacaaggtggtttactggacagacatcagcgagccttccattgggagagccagcctccacggtggagagccaaccaccatcattcgacaagatcttggaagccctgaaggcattgcccttgaccatcttggtcgaaccatcttctggacggactctcagttggatcgaatagaagttgcaaagatggatggcacccagcgccgagtgctgtttgacacgggtttggtgaatcccagaggcattgtgacagaccccgtaagagggaacctttattggacagattggaacagagataatcccaaaattgagacttctcacatggatggcaccaaccggaggattctcgcacaggacaacctgggcttgcccaatggtctgacctttgatgcattctcatctcagctttgctgggtggatgcaggcacccatagggcagaatgcctgaacccagctcagcctggcagacgcaaagttctcgaagggctccagtatcctttcgctgtgactagctatgggaagaatttgtactacacagactggaagacgaattcagtgattgccatggaccttgctatatccaaagagatggataccttccacccacacaagcagacccggctatatggcatcaccatcgccctgtcccagtgtccccaaggccacaattactgctcagtgaataatggtggatgtacccacctctgcttgcccactccagggagcaggacctgccgatgtcctgacaacaccctgggagttgactgcattgaacggaaatga

SEQ ID NO: 2 - DNA, 20 bp, Artificial Sequence
  F1noG2 1F
ctgggtcactgtcaccctgg

SEQ ID NO: 3 - DNA, 30 bp, Artificial Sequence
  noG2 2R
atggattctgaagacagacaccagagacac

SEQ ID NO: 4 - DNA, 29 bp, Artificial Sequence
  no G2 2F
ctggtgtctgtcttcagaatccatgctac

SEQ ID NO: 5 - DNA, 24 bp, Artificial Sequence
  F1 no G2 1R
gaaggcacagtcgaggctgatcag

SEQ ID NO: 6 - DNA, 30 bp, Artificial Sequence
  Bam shnoG2 1F
cggcagcctgaatgaggatccatgcataga

SEQ ID NO: 7 - DNA, 25 bp, Artificial Sequence
  shnoG2 2R
cacagtagttgatgggacagacacc

SEQ ID NO: 8 - DNA, 26 bp, Artificial Sequence
  shnoG2 2F
gtctctggtgtctgtcccatcaacta

SEQ ID NO: 9 - DNA, 30 bp, Artificial Sequencce
  sse shnoG2 1R
gaggcacaaacatcccctgcagggtgggcc

SEQ ID NO: 10 - DNA, 4639 bp, Artificial Sequence
  pAAV-MCS Expression Vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatcctctagagtcgacctgcagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 130 bp, Artificial Sequence
  5′ ITR
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 12 - DNA, 663 bp, Artificial Sequence
  CMV Promoter
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 13 - DNA, 493 bp, Artificial Sequence
  Human beta globin Intron
cgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggat

SEQ ID NO: 14 - DNA, 76 bp, Artificial Sequence
  MCS
atcgattgaattccccggggatcctctagagtcgacctgcagaagcttgcctcgagcagcgctgctcgagagatct

SEQ ID NO: 15 - DNA, 479 bp, Artificial Sequence
  PolyA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 16 - DNA, 141 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 7333 bp, Artificial Sequence
  pAAV-DJ Vector
ccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcag

SEQ ID NO: 18 - DNA, 1866 bp, Artificial Sequence
  AAV-2 Rep gene
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 19 - DNA, 2214 bp, Artificial Sequence
  AAV-DJ Cap gene
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 20 - DNA, 11635 bp, Artificial Sequence
  pHelper Vector
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 21 - protein, 1002 aa, Artificial Sequence
  Mouse alphaLNNdDeltaG2
MRAWIFFLLCLAGRALAQQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGGMCICYGHASSCPWDEEAKQLQCQCEHNTCGESCDRCCPGYHQQPWRPGTISSGNECEECNCHNKAKDCYYDSSVAKERRSLNTAGQYSGGGVCVNCSQNTTGINCETCIDQYYRPHKVSPYDDHPCRPCNCDPVGSLSSVCIKDDRHADLANGKWPGQCPCRKGYAGDKCDRCQFGYRGFPNCIPCDCRTVGSLNEDPCIEPCLCKKNVEGKNCDRCKPGFYNLKERNPEGCSECFCFGVSGVCPINYCETGLHNCDIPQRAQCIYMGGSSYTCSCLPGFSGDGRACRDVDECQHSRCHPDAFCYNTPGSFTCQCKPGYQGDGFRCMPGEVSKTRCQLEREHILGAAGGADAQRPTLQGMFVPQCDEYGHYVPTQCHHSTGYCWCVDRDGRELEGSRTPPGMRPPCLSTVAPPIHQGPVVPTAVIPLPPGTHLLFAQTGKIERLPLERNTMKKTERKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPHKQTRLYGITIALSQCPQGHNYCSVNNGGCTHLCLPTPGSRTCRCPDNTLGVDCIERK

SEQ ID NO: 22 - protein, 1002 aa, Artificial Sequence
  Human shNoG2 amino acid sequence; Human alphaLNNDdDeltaG2
MRAWIFFLLCLAGRALARQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 23 - protein, 969 aa, Artificial Sequence
  mouse miniagrin sequence for AAV
MVRPRLSFPAPLLPLLLLLAAAAPAVPGASGTCPERALERREEEANVVLTGTVEEILNVDPVQHTYSCKVRVWRYLKGKDVVAQESLLDGGNKVVIGGFGDPLICDNQVSTGDTRIFFVNPAPPYLWPAHKNELMLNSSLMRITLRNLEEVEFCVEDKPGIHFTAAPSMPPDVCRGMLCGFGAVCEPSVEDPGRASCVCKKNVCPAMVAPVCGSDASTYSNECELQRAQCNQQRRIRLLRQGPCPPKSCDSQPCLHGGTCQDLDSGKGFSCSCTAGRAGTVCEKVQLPSVPAFKGHSFLAFPTLRAYHTLRLALEFRALETEGLLLYNGNARGKDFLALALLDGHVQFRFDTGSGPAVLTSLVPVEPGRWHRLELSRHWRQGTLSVDGEAPVVGESPSGTDGLNLDTKLYVGGLPEEQVATVLDRTSVGIGLKGCIRMLDINNQQLELSDWQRAVVQSSGVGECGDHPCSPNPCHGGALCQALEAGVFLCQCPPGRFGPTCADEKNPCQPNPCHGSAPCHVLSRGGAKCACPLGRSGSFCETVLENAGSRPFLADFNGFSYLELKGLHTFERDLGEKMALEMVFLARGPSGLLLYNGQKTDGKGDFVSLALHNRHLEFRYDLGKGAAIIRSKEPIALGTWVRVFLERNGRKGALQVGDGPRVLGESPVPHTMLNLKEPLYVGGAPDFSKLARGAAVASGFDGAIQLVSLRGHQLLTQEHVLRAVDVAPFAGHPCTQAVDNPCLNGGSCIPREATYECLCPGGFSGLHCEKGIVEKSVGDLETLAFDGRTYIEYLNAVTESEKALQSNHFELSLRTEATQGLVLWIGKVGERADYMALAIVDGHLQLSYDLGSQPVVLRSTVKVNTNRWLRVRAHREHREGSLQVGNEAPVTGSSPLGATQLDTDGALWLGGLQKLPVGQALPKAYGTGFVGCLRDVVVGHRQLHLLEDAVTKPELRPCPTLIDGSGKAM

SEQ ID NO: 24 - DNA, 3009 bp, Artificial Sequence
  Human shNoG2 nucleotide sequence
atgagggcctggatcttctttctcctttgcctggccgggagggctctggcacggcagagaggcctgtttcctgccattctcaatcttgccagcaatgctcacatcagcaccaatgccacctgtggcgagaaggggccggagatgttctgcaaacttgtggagcatgtgccaggtcggcccgtccgaaacccacagtgccggatctgtgatggcaacagcgcaaaccccagagaacgccatccaatatcacatgccatagatggcaccaataactggtggcaaagtcccagcattcagaatgggagagaatatcactgggtcacaatcactctggacttaagacaggtctttcaagttgcatatgtcatcattaaagctgccaatgcccctcgacctggaaactggattttggagcgttctctggatggcaccacgttcagcccctggcagtattatgcagtcagcgactcagagtgtttgtctcgttacaatataactccaagacgagggccacccacctacagggctgatgatgaagtgatctgcacctcctattattccagattggtgccacttgagcatggagagattcatacatcactcatcaatggcagaccaagcgctgacgatctttcacccaagttgttggaattcacttctgcacgatatattcgccttcgcttgcaacgcattagaacgctcaatgcagatctcatgacccttagccaccgggaacctaaagaactggatcctattgttaccagacgctattattattcaataaaggacatttctgttggaggcatgtgtatctgctatggccatgctagtagctgcccatgggatgaaactacaaagaaactgcagtgtcaatgtgagcataatacttgcggggagagctgtaacaggtgctgtcctgggtaccatcagcagccctggaggccgggaaccgtgtcctccggcaatacatgtgaagcatgtaattgtcacaataaagccaaagactgttactatgatgaaagtgttgcaaagcagaagaaaagtttgaatactgctggacagttcagaggaggaggggtttgcataaattgcttgcagaacaccatgggaatcaactgtgaaacctgtattgatggatattatagaccacacaaagtgtctccttatgaggatgagccttgccgcccctgtaattgtgaccctgtggggtccctcagttctgtctgtattaaggatgacctccattctgacttacacaatgggaagcagccaggtcagtgcccatgtaaggaaggttatacaggagaaaaatgtgatcgctgccaacttggctataaggattacccgacctgtgtctcctgtgggtgcaacccagtgggcagtgccagtgatgagccctgcacagggccctgtgtttgtaaggaaaacgttgaggggaaggcctgtgatcgctgcaagccaggattctataacttgaaggaaaaaaacccccggggctgctccgagtgcttctgctttggcgtttctgatgtctgccccatcaactactgtgaaactggccttcataactgcgacataccccagcgggcccagtgtatctacacaggaggctcctcctacacctgttcctgcttgccaggcttttctggggatggccaagcctgccaagatgtagatgaatgccagccaagccgatgtcaccctgacgccttctgctacaacactccaggctctttcacgtgccagtgcaaacctggttatcagggagacggcttccgttgcgtgcccggagaggtggagaaaacccggtgccagcacgagcgagaacacattctcggggcagcgggggcgacagacccacagcgacccattcctccggggctgttcgttcctgagtgcgatgcgcacgggcactacgcgcccacccagtgccacggcagcaccggctactgctggtgcgtggatcgcgacggccgcgaggtggagggcaccaggaccaggcccgggatgacgcccccgtgtctgagtacagtggctcccccgattcaccaaggacctgcggtgcctaccgccgtgatccccttgcctcctgggacccatttactctttgcccagactgggaagattgagcgcctgcccctggagggaaataccatgaggaagacagaagcaaaggcgttccttcatgtcccggctaaagtcatcattggactggcctttgactgcgtggacaagatggtttactggacggacatcactgagccttccattgggagagctagtctacatggtggagagccaaccaccatcattagacaagatcttggaagtccagaaggtatcgctgttgatcaccttggccgcaacatcttctggacagactctaacctggatcgaatagaagtggcgaagctggacggcacgcagcgccgggtgctctttgagactgacttggtgaatcccagaggcattgtaacggattccgtgagagggaacctttactggacagactggaacagagataaccccaagattgaaacttcctacatggacggcacgaaccggaggatccttgtgcaggatgacctgggcttgcccaatggactgaccttcgatgcgttctcatctcagctctgctgggtggatgcaggcaccaatcgggcggaatgcctgaaccccagtcagcccagcagacgcaaggctctcgaagggctccagtatccttttgctgtgacgagctacgggaagaatctgtatttcacagactggaagatgaattccgtggttgctctcgatcttgcaatttccaaggagacggatgctttccaaccccacaagcagacccggctgtatggcatcaccacggccctgtctcagtgtccgcaaggccataactactgctcagtgaacaatggcggctgcacccacctatgcttggccaccccagggagcaggacctgccgttgccctgacaacaccttgggagttgactgtatcgaacagaaatga

SEQ ID NO: 25 - DNA, 51 bp, Artificial Sequence
  Mouse BM-40 (Sparc) signal sequence [DNA, 51 bp)
atgagggcctggatcttctttctcctttgcctggccgggagggctctggca

SEQ ID NO: 26 - protein, 17 aa, Artificial Sequence
  Mouse BM-40 (Sparc) signal peptide
MRAWIFFLLCLAGRALA

SEQ ID NO: 27 - DNA, 72 bp, Artificial Sequence
  Mouse Lm  1 endogenous signal sequence [DNA, 72 bp]
atgcgcggcagcggcacgggagccgcgctcctggtgctcctggcctcggtgctctgggtcaccgtgcggagc

SEQ ID NO: 28 - protein, 24 aa, Artificial Sequence
  Mouse laminin  1 endogenous signal peptide
MRGSGTGAALLVLLASVLWVTVRS

SEQ ID NO: 29 - DNA, 51 bp, Artificial Sequence; identical to SEQ ID NO: 25
  Laminin (Lm)  1 signal peptide [DNA, 51 bp]

SEQ ID NO: 30 - protein, 17 aa, Artificial Sequence
  Human laminin  1 signal peptide
MRAWIFFLLCLAGRALA

SEQ ID NO: 31 - DNA, 753 bp, Artificial Sequence
  Mouse Lm  1 LN domain [DNA, 753 bp]
cagcagagaggcttgttccctgccattctcaacctggccaccaatgcccacatcagcgccaatgctacctgtggagagaaggggcctgagatgttctgcaaactcgtggagcacgtgccgggccggcctgttcgacacgcccaatgccgggtctgtgacggtaacagtacgaatcctagagagcgccatccgatatcacacgcaatcgatggcaccaacaactggtggcagagccccagtattcagaatgggagagagtatcactgggtcactgtcaccctggacttacggcaggtctttcaagttgcatacatcatcattaaagctgccaatgcccctcggcctggaaactggattttggagcgctccgtggatggcgtcaagttcaaaccctggcagtactatgccgtcagcgatacagagtgtttgacccgctacaaaataactccacggcggggacctcccacttacagagcagacaacgaagtcatctgcacctcgtattattcaaagctggtgccacttgaacatggagagattcacacatcactcatcaatggcagacccagcgctgacgacccctcaccccagttgctggaattcacctcagcacggtacattcgccttcgtcttcagcgcatcagaacactcaacgcagacctcatgacccttagccatcgggacctcagagaccttgaccccattgtcacaagacgttattactattcgataaaagacatttccgttggaggc

SEQ ID NO: 32 - protein, 251 aa, Artificial Sequence
  Mouse Lm  1 LN  [polymerization domain]
QQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGG

SEQ ID NO: 33 - DNA, 1020 bp, Artificial Sequence
  Human Lm 1 LN [DNA, 753 bp]
cggcagagaggcctgtttcctgccattctcaatcttgccagcaatgctcacatcagcaccaatgccacctgtggcgagaaggggccggagatgttctgcaaacttgtggagcatgtgccaggtcggcccgtccgaaacccacagtgccggatctgtgatggcaacagcgcaaaccccagagaacgccatccaatatcacatgccatagatggcaccaataactggtggcaaagtcccagcattcagaatgggagagaatatcactggcggcagagaggcctgtttcctgccattctcaatcttgccagcaatgctcacatcagcaccaatgccacctgtggcgagaaggggccggagatgttctgcaaacttgtggagcatgtgccaggtcggcccgtccgaaacccacagtgccggatctgtgatggcaacagcgcaaaccccagagaacgccatccaatatcacatgccatagatggcaccaataactggtggcaaagtcccagcattcagaatgggagagaatatcactgggtcacaatcactctggacttaagacaggtctttcaagttgcatatgtcatcattaaagctgccaatgcccctcgacctggaaactggattttggagcgttctctggatggcaccacgttcagcccctggcagtattatgcagtcagcgactcagagtgtttgtctcgttacaatataactccaagacgagggccacccacctacagggctgatgatgaagtgatctgcacctcctattattccagattggtgccacttgagcatggagagattcatacatcactcatcaatggcagaccaagcgctgacgatctttcacccaagttgttggaattcacttctgcacgatatattcgccttcgcttgcaacgcattagaacgctcaatgcagatctcatgacccttagccaccgggaacctaaagaactggatcctattgttaccagacgctattattattcaataaaggacatttctgttggaggc

SEQ ID NO: 34 - protein, 251 aa, Artificial Sequence
  Human Lm 1 LN
RQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGG

SEQ ID NO: 35 - DNA, 171 bp, Artificial Sequence
  Mouse Lm  1 LEa-1 domain [DNA, 171 bp]
atgtgcatttgctacggccatgccagcagctgcccgtgggatgaagaagcaaagcaactacagtgtcagtgtgaacacaatacgtgtggcgagagctgcgacaggtgctgtcctggctaccatcagcagccctggaggcccggaaccatttcctccggcaacgagtgtgag

SEQ ID NO: 36 - protein, 57 aa, Artificial Sequence
  Mouse Lm  1 LEa-1 [required for LN folding; spacer domain]
MCICYGHASSCPWDEEAKQLQCQCEHNTCGESCDRCCPGYHQQPWRPGTISSGNECE

SEQ ID NO: 37 - DNA, 171 bp, Artificial Sequence
  Human Lm 1 LEa-1 [DNA, 171 bp]
atgtgtatctgctatggccatgctagtagctgcccatgggatgaaactacaaagaaactgcagtgtcaatgtgagcataatacttgcggggagagctgtaacaggtgctgtcctgggtaccatcagcagccctggaggccgggaaccgtgtcctccggcaatacatgtgaa

SEQ ID NO: 38 - protein, 57 aa, Artificial Sequence
  Human Lm 1 LEa-1
MCICYGHASSCPWDETTKKLQCQCEHNTCGESCNRCCPGYHQQPWRPGTVSSGNTCE

SEQ ID NO: 39 - DNA, 210 bp, Artificial Sequence
  Mouse Lm  1 LEa-2 domain [DNA, 210 bp]
gaatgcaactgtcacaacaaagccaaagattgttactatgacagcagtgttgcaaaggagaggagaagcctgaacactgccgggcagtacagtggaggaggggtttgtgtcaactgctcgcagaataccacagggatcaactgtgaaacctgtatcgaccagtattacagacctcacaaggtatctccttatgatgaccacccttgccgt

SEQ ID NO: 40 - protein, 70 aa, Artificial Sequence
  Mouse Lm  1 LEa-2 [required for LN folding; spacer domain]
ECNCHNKAKDCYYDSSVAKERRSLNTAGQYSGGGVCVNCSQNTTGINCETCIDQYYRPHKVSPYDDHPCR

SEQ ID NO: 41 - DNA, 210 bp, Artificial Sequence
  Human Lm 1 LEa-2 [DNA, 210 bp]
gcatgtaattgtcacaataaagccaaagactgttactatgatgaaagtgttgcaaagcagaagaaaagtttgaatactgctggacagttcagaggaggaggggtttgcataaattgcttgcagaacaccatgggaatcaactgtgaaacctgtattgatggatattatagaccacacaaagtgtctccttatgaggatgagccttgccgc

SEQ ID NO: 42 - protein, 70 aa, Artificial Sequence
  Human Lm 1 LEa-2
ACNCHNKAKDCYYDESVAKQKKSLNTAGQFRGGGVCINCLQNTMGINCETCIDGYYRPHKVSPYEDEPCR

SEQ ID NO: 43 - DNA, 171 bp, Artificial Sequence
  Mouse Lm  1 LEa-3 domain [DNA, 171 bp]
ccctgtaactgtgaccctgtggggtctctgagttctgtctgtatcaaggatgaccgccatgccgatttagccaatggaaagtggccaggtcagtgtccatgtaggaaaggttatgctggagataaatgtgaccgctgccagtttggctaccggggtttcccaaattgcatc

SEQ ID NO: 44 - protein, 57 aa, Artificial Sequence
  Mouse Lm  1 LEa-3 [domain acting as spacer]
PCNCDPVGSLSSVCIKDDRHADLANGKWPGQCPCRKGYAGDKCDRCQFGYRGFPNCI

SEQ ID NO: 45 - DNA, 171 bp, Artificial Sequence
  Human Lm 1 LEa-3 [DNA, 171 bp]
ccctgtaattgtgaccctgtggggtccctcagttctgtctgtattaaggatgacctccattctgacttacacaatgggaagcagccaggtcagtgcccatgtaaggaaggttatacaggagaaaaatgtgatcgctgccaacttggctataaggattacccgacctgtgtc

SEQ ID NO: 46 - protein, 57 aa, Artificial Sequence
  Human Lm 1 LEa-3
PCNCDPVGSLSSVCIKDDLHSDLHNGKQPGQCPCKEGYTGEKCDRCQLGYKDYPTCV

SEQ ID NO: 47 - DNA, 147 bp, Artificial Sequence
  Mouse Lm  1 LEa-4 domain [DNA, 147 bp]
ccctgtgactgcaggactgtcggcagcctgaatgaggatccatgcatagagccgtgtctttgtaagaaaaatgttgagggtaagaactgtgatcgctgcaagccaggattctacaacttgaaggaacgaaaccccgagggctgctcc

SEQ ID NO: 48 - protein, 49 aa, Artificial Sequence
  Mouse Lm  1 LEa-4 [spacer domain]
PCDCRTVGSLNEDPCIEPCLCKKNVEGKNCDRCKPGFYNLKERNPEGCS

SEQ ID NO: 49 - DNA, 147 bp, Artificial Sequence
  Human Lm 1 LEa-4 [DNA, 147 bp]
tcctgtgggtgcaacccagtgggcagtgccagtgatgagccctgcacagggccctgtgtttgtaaggaaaacgttgaggggaaggcctgtgatcgctgcaagccaggattctataacttgaaggaaaaaaacccccggggctgctcc

SEQ ID NO: 50 - protein, 49 aa, Artificial Sequence
  Human Lm 1 LEa-4
SCGCNPVGSASDEPCTGPCVCKENVEGKACDRCKPGFYNLKEKNPRGCS

SEQ ID NO: 51 - DNA, 33 bp, Artificial Sequence
  Mouse Lm  1 LF domain LE-type fragment with 3 cys [DNA, 33 bp]
gagtgcttctgcttcggtgtctctggtgtctgt

SEQ ID NO: 52 - protein, 11 aa, Artificial Sequence
  Mouse Lm  1 LF fragment (with 3 cys) [spacer segment]
ECFCFGVSGVC

SEQ ID NO: 53 - DNA, 33 bp, Artificial Sequence
  Human Lm 1 LF fragment (with 3 cys)[DNA, 33 bp]
gagtgcttctgctttggcgtttctgatgtctgc

SEQ ID NO: 54 - protein, 10 aa, Artificial Sequence
  Human Lm 1 LF fragment (with 3 cys)
CFCFGVSDVC

SEQ ID NO: 55 - DNA, 843 bp, Artificial Sequence
  Mouse Nidogen-1 G2 domain [DNA, 843 bp]
cagcagacttgtgccaacaatagacaccagtgctccgtgcatgcagagtgcagagactatgctactggcttctgctgcaggtgtgtggccaactacacaggcaatggcagacagtgcgtggcagaaggctctccacaacgggtcaatggcaaggtgaagggaaggatcttcgtggggagcagccaggtccccgtggtgtttgagaacactgacctgcactcctatgtggtgatgaaccacgggcgctcttacacagccatcagcaccatccctgaaaccgtcggctactctctgctccccctggcacccattggaggcatcatcggatggatgtttgcagtggagcaggatgggttcaagaatgggtttagcatcactgggggcgagtttacccggcaagctgaggtgaccttcctggggcacccaggcaagctggtcctgaagcagcagttcagcggtattgatgaacatggacacctgaccatcagcacggagctggagggccgcgtgccgcagatcccctatggagcctcggtgcacattgagccctacaccgaactgtaccactactccagctcagtgatcacttcctcctccacccgggagtacacggtgatggagcctgatcaggacggcgctgcaccctcacacacccatatttaccagtggcgtcagaccatcaccttccaggagtgtgcccacgatgacgccaggccagccctgcccagcacccagcagctctctgtggacagcgtgtttgtcctgtacaacaaggaggagaggatcttgcgctatgccctcagcaactccatcgggcctgtgagggatggctcccctgatgcc

SEQ ID NO: 56 - protein, 281 aa, Artificial Sequence
  Mouse Nidogen-1 G2 domain [direct collagen-IV, perlecan binding]
QQTCANNRHQCSVHAECRDYATGFCCRCVANYTGNGRQCVAEGSPQRVNGKVKGRIFVGSSQVPVVFENTDLHSYVVMNHGRSYTAISTIPETVGYSLLPLAPIGGIIGWMFAVEQDGFKNGFSITGGEFTRQAEVTFLGHPGKLVLKQQFSGIDEHGHLTISTELEGRVPQIPYGASVHIEPYTELYHYSSSVITSSSTREYTVMEPDQDGAAPSHTHIYQWRQTITFQECAHDDARPALPSTQQLSVDSVFVLYNKEERILRYALSNSIGPVRDGSPDA

SEQ ID NO: 57 - DNA, 843 bp, Artificial Sequence
  Human Nidogen-1 G2 domain (direct collagen-IV, perlecan binding)[DNA, 843 bp]
cgccagacgtgtgctaacaacagacaccagtgctcggtgcacgcagagtgcagggactacgccacgggcttctgctgcagctgtgtcgctggctatacgggcaatggcaggcaatgtgttgcagaaggttccccccagcgagtcaatggcaaggtgaaaggaaggatctttgtggggagcagccaggtccccattgtctttgagaacactgacctccactcttacgtagtaatgaaccacgggcgctcctacacagccatcagcaccattcccgagaccgttggatattctctgcttccactggccccagttggaggcatcattggatggatgtttgcagtggagcaggacggattcaagaatgggttcagcatcaccgggggtgagttcactcgccaggctgaggtgaccttcgtggggcacccgggcaatctggtcattaagcagcggttcagcggcatcgatgagcatgggcacctgaccatcgacacggagctggagggccgcgtgccgcagattccgttcggctcctccgtgcacattgagccctacacggagctgtaccactactccacctcagtgatcacttcctcctccacccgggagtacacggtgactgagcccgagcgagatggggcatctccttcacgcatctacacttaccagtggcgccagaccatcaccttccaggaatgcgtccacgatgactcccggccagccctgcccagcacccagcagctctcggtggacagcgtgttcgtcctgtacaaccaggaggagaagatcttgcgctatgctctcagcaactccattgggcctgtgagggaaggctcccctgatgct

SEQ ID NO: 58 - protein, 281 aa, Artificial Sequence
  Human Nidogen-1 G2 domain (direct collagen-IV, perlecan binding)
RQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDA

SEQ ID NO: 59 - DNA, 126 bp, Artificial Sequence
  Mouse Nidogen-1 EGF-like 2 domain [126 bp]
cttcagaatccatgctacattggcacccatgggtgtgacagcaatgctgcctgtcgccctggccctggaacacagttcacctgcgaatgctccatcggcttccgaggagacgggcagacttgctat

SEQ ID NO: 60 - protein, 42 aa, Artificial Sequence
  Mouse Nidogen-1 EGF-like 2 [spacer]
LQNPCYIGTHGCDSNAACRPGPGTQFTCECSIGFRGDGQTCY

SEQ ID NO: 61 - DNA, 126 bp, Artificial Sequence
  Human Nidogen-1 EGF-like 2 domain [DNA, 126 bp]
cttcagaatccctgctacatcggcactcatgggtgtgacaccaacgcggcctgtcgccctggtcccaggacacagttcacctgcgagtgctccatcggcttccgaggagacgggcgaacctgctat

SEQ ID NO: 62 - protein, 42 aa, Artificial Sequence
  Human Nidogen-1 EGF-like 2 domain
LQNPCYIGTHGCDTNAACRPGPRTQFTCECSIGFRGDGRTCY

SEQ ID NO: 63 - DNA, 126 bp, Artificial Sequence
  Mouse Niogen-1 EGF-like 3 domain [126 bp]
gatattgatgagtgttcagagcagccttcccgctgtgggaaccatgcggtctgcaacaacctcccaggaaccttccgctgcgagtgtgtagagggctaccacttctcagacaggggaacatgcgtg

SEQ ID NO: 64 - protein, 42 aa, Artificial Sequence
  Mouse Nidogen-1 EGF-like 3
DIDECSEQPSRCGNHAVCNNLPGTFRCECVEGYHFSDRGTCV

SEQ ID NO: 65 - DNA, 126 bp, Artificial Sequence; identical to SEQ ID NO: 61
  Human Nidogen 1 EGF like 3 domain DNA 126 bp

SEQ ID NO: 66 - protein, 42 aa, Artificial Sequence; identical to SEQ ID NO: 62
  Human Nidogen-1 EGF-like 3 domain

SEQ ID NO: 67 - DNA, 18 bp, Artificial Sequence
  Mouse Nidogen-1 spacer segment between EGF-3 and -4 [DNA, 18 bp]
gctgccgaggaccaacgt

SEQ ID NO: 68 - protein, 6 aa, Artificial Sequence
  Mouse Nidogen-1 spacer segment between EGF-3 and -4
AAEDQR

SEQ ID NO: 69 - DNA, 18 bp, Artificial Sequence
  Human Nidogen-1 spacer segment between EGF-3 and -4 [DNA, 18 bp]
gctgtcgtggaccagcgc

SEQ ID NO: 70 - protein, 6 aa, Artificial Sequence
  Human Nidogen-1 spacer segment between EGF-3 and -4
AVVDQR

SEQ ID NO: 71 - DNA, 132 bp, Artificial Sequence
  Mouse Nidogen-1 EGF-like 4 domain [132 bp]
cccatcaactactgtgaaactggtctccacaactgtgatatcccccagcgagcccagtgcatctatatgggtggttcctcctacacctgctcctgtctgcctggcttctctggggatggcagagcctgccga

SEQ ID NO: 72 - protein, 44 aa, Artificial Sequence
  Mouse Nidogen-1 EGF-like 4
PINYCETGLHNCDIPQRAQCIYMGGSSYTCSCLPGFSGDGRACR

SEQ ID NO: 73 - DNA, 132 bp, Artificial Sequence
  Human Nidogen-1 EGF-like 4 domain [DNA, 132 bp]
cccatcaactactgtgaaactggccttcataactgcgacataccccagcgggcccagtgtatctacacaggaggctcctcctacacctgttcctgcttgccaggcttttctggggatggccaagcctgccaa

SEQ ID NO: 74 - protein, 44 aa, Artificial Sequence
  Human Nidogen-1 EGF-like 4 domain
PINYCETGLHNCDIPQRAQCIYTGGSSYTCSCLPGFSGDGQACQ

SEQ ID NO: 75 - DNA, 141 bp, Artificial Sequence
  Mouse Nidogen-1 EGF-like 5 domain [DNA, 141 bp]
gacgtggatgaatgccagcacagccgatgtcaccccgatgccttctgctacaacacaccaggctctttcacatgtcagtgcaagcctggctatcagggggatggcttccgatgcatgcccggagaggtgagcaaaacccgg

SEQ ID NO: 76 - protein, 47 aa, Artificial Sequence
  Mouse Nidogen-1 EGF-like 5 [spacer]
DVDECQHSRCHPDAFCYNTPGSFTCQCKPGYQGDGFRCMPGEVSKTR

SEQ ID NO: 77 - DNA, 141 bp, Artificial Sequence
  Human Nidogen-1 EGF-like 5 domain [DNA, 141 bp]
gatgtagatgaatgccagccaagccgatgtcaccctgacgccttctgctacaacactccaggctctttcacgtgccagtgcaaacctggttatcagggagacggcttccgttgcgtgcccggagaggtggagaaaacccgg

SEQ ID NO: 78 - protein, 47 aa, Artificial Sequence
  Human Nidogen-1 EGF-like 5 domain
DVDECQPSRCHPDAFCYNTPGSFTCQCKPGYQGDGFRCVPGEVEKTR

SEQ ID NO: 79 - DNA, 282 bp, Artificial Sequence
  Mouse Nidogen-1 G3 TY (thyroglobulin-like) domain [DNA, 282 bp]
tgtcaactggaacgagagcacatccttggagcagccggcggggcagatgcacagcggcccaccctgcaggggatgtttgtgcctcagtgtgatgaatatggacactatgtacccacccagtgtcaccacagcactggctactgctggtgtgtggaccgagatggtcgggagctggagggtagccgtaccccacctgggatgaggcccccgtgtctgagtacagtggctcctcctattcaccagggaccagtagtacctacagctgtcatccccctgcctcca

SEQ ID NO: 80 - protein, 94 aa, Artificial Sequence
  Mouse Nidogen “G3” TY (thyroglobulin-like) domain
CQLEREHILGAAGGADAQRPTLQGMFVPQCDEYGHYVPTQCHHSTGYCWCVDRDGRELEGSRTPPGMRPPCLSTVAPPIHQGPVVPTAVIPLPP

SEQ ID NO: 81 - DNA, 282 bp, Artificial Sequence
  Human Nidogen-1 G3 TY (thyroglobulin-like) domain [DNA, 282 bp]
tgccagcacgagcgagaacacattctcggggcagcgggggcgacagacccacagcgacccattcctccggggctgttcgttcctgagtgcgatgcgcacgggcactacgcgcccacccagtgccacggcagcaccggctactgctggtgcgtggatcgcgacggccgcgaggtggagggcaccaggaccaggcccgggatgacgcccccgtgtctgagtacagtggctcccccgattcaccaaggacctgcggtgcctaccgccgtgatccccttgcctcct

SEQ ID NO: 82 - protein, 94 aa, Artificial Sequence
  Human Nidogen-1 G3 TY (thyroglobulin-like) domain
CQHEREHILGAAGATDPQRPIPPGLFVPECDAHGHYAPTQCHGSTGYCWCVDRDGREVEGTRTRPGMTPPCLSTVAPPIHQGPAVPTAVIPLPP

SEQ ID NO: 83 - DNA, 744 bp, Artificial Sequence
  Mouse Nidogen-1 G3  -Propeller domain [DNA, 744 bp]
gggacacacttactctttgctcagactggaaagattgaacgcctgcccctggaaagaaacaccatgaagaagacagaacgcaaggcctttctccatatccctgcaaaagtcatcattggactggcctttgactgcgtggacaaggtggtttactggacagacatcagcgagccttccattgggagagccagcctccacggtggagagccaaccaccatcattcgacaagatcttggaagccctgaaggcattgcccttgaccatcttggtcgaaccatcttctggacggactctcagttggatcgaatagaagttgcaaagatggatggcacccagcgccgagtgctgtttgacacgggtttggtgaatcccagaggcattgtgacagaccccgtaagagggaacctttattggacagattggaacagagataatcccaaaattgagacttctcacatggatggcaccaaccggaggattctcgcacaggacaacctgggcttgcccaatggtctgacctttgatgcattctcatctcagctttgctgggtggatgcaggcacccatagggcagaatgcctgaacccagctcagcctggcagacgcaaagttctcgaagggctccagtatcctttcgctgtgactagctatgggaagaatttgtactacacagactggaagacgaattcagtgattgccatggaccttgctatatccaaagagatggataccttccacccacac

SEQ ID NO: 84 - protein, 248 aa, Artificial Sequence
  Mouse Nidogen “G3”  -Propeller [laminin-binding domain]
GTHLLFAQTGKIERLPLERNTMKKTERKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPH

SEQ ID NO: 85 - DNA, 744 bp, Artificial Sequence
  Human Nidogen-1 G3  -Propeller domain [DNA, 744 bp]
gggacccatttactctttgcccagactgggaagattgagcgcctgcccctggagggaaataccatgaggaagacagaagcaaaggcgttccttcatgtcccggctaaagtcatcattggactggcctttgactgcgtggacaagatggtttactggacggacatcactgagccttccattgggagagctagtctacatggtggagagccaaccaccatcattagacaagatcttggaagtccagaaggtatcgctgttgatcaccttggccgcaacatcttctggacagactctaacctggatcgaatagaagtggcgaagctggacggcacgcagcgccgggtgctctttgagactgacttggtgaatcccagaggcattgtaacggattccgtgagagggaacctttactggacagactggaacagagataaccccaagattgaaacttcctacatggacggcacgaaccggaggatccttgtgcaggatgacctgggcttgcccaatggactgaccttcgatgcgttctcatctcagctctgctgggtggatgcaggcaccaatcgggcggaatgcctgaaccccagtcagcccagcagacgcaaggctctcgaagggctccagtatccttttgctgtgacgagctacgggaagaatctgtatttcacagactggaagatgaattccgtggttgctctcgatcttgcaatttccaaggagacggatgctttccaaccccac

SEQ ID NO: 86 - protein, 248 aa, Artificial Sequence
  Human Nidogen-1 G3  -Propeller domain
GTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPH

SEQ ID NO: 87 - DNA, 171 bp, Artificial Sequence
  Mouse Nidogen-1 G3 EGF-like 6 domain [DNA, 171 bp]
aagcagacccggctatatggcatcaccatcgccctgtcccagtgtccccaaggccacaattactgctcagtgaataatggtggatgtacccacctctgcttgcccactccagggagcaggacctgccgatgtcctgacaacaccctgggagttgactgcattgaacggaaa

SEQ ID NO: 88 - protein, 57 aa, Artificial Sequence
  Mouse Nidogen “G3” EGF-like 6 [contacts laminin LE surface]
KQTRLYGITIALSQCPQGHNYCSVNNGGCTHLCLPTPGSRTCRCPDNTLGVDCIERK

SEQ ID NO: 89 - DNA, 162 bp, Artificial Sequence
  Human Nidogen-1 G3 EGF-like 6 domain [DNA, 162 bp]
aagcagacccggctgtatggcatcaccacggccctgtctcagtgtccgcaaggccataactactgctcagtgaacaatggcggctgcacccacctatgcttggccaccccagggagcaggacctgccgttgccctgacaacaccttgggagttgactgtatc

SEQ ID NO: 90 - protein, 54 aa, Artificial Sequence
  Human Nidogen-1 G3 EGF-like 6 domain
KQTRLYGITTALSQCPQGHNYCSVNNGGCTHLCLATPGSRTCRCPDNTLGVDCI

SEQ ID NO: 91 - DNA, 63 bp, Artificial Sequence
  Mouse Laminin  1 signal peptide [63 bp]
atggggctgctccaggtgttcgcctttggtgtcctagccctatggggcacccgagtgtgcgct

SEQ ID NO: 92 - protein, 21 aa, Artificial Sequence
  Mouse Laminin  1 signal peptide
MGLLQVFAFGVLALWGTRVCA

SEQ ID NO: 93 - DNA, 63 bp, Artificial Sequence
  Human Laminin  1 signal [63 bp]
atggggcttctccagttgctagctttcagtttcttagccctgtgcagagcccgagtgcgcgct

SEQ ID NO: 94 - protein, 21 aa, Artificial Sequence
  Human Laminin  1 signal peptide
MGLLQLLAFSFLALCRARVRA

SEQ ID NO: 95 - DNA, 744 bp, Artificial Sequence
  Mouse Laminin  1 LN domain [744 bp]
caggaaccggagttcagctatggctgcgcagaaggcagctgctaccctgccactggcgaccttctcatcggccgagcgcaaaagctctccgtgacttcgacatgtggactgcacaaaccagagccctactgtattgttagccacctgcaggaggacaagaaatgcttcatatgtgactcccgagacccttatcacgagaccctcaaccccgacagccatctcattgagaacgtggtcaccacatttgctccaaaccgccttaagatctggtggcaatcggaaaatggtgtggagaacgtgaccatccaactggacctggaagcagaattccatttcactcatctcatcatgaccttcaagacattccgcccagccgccatgctgatcgagcggtcttctgactttgggaagacttggggcgtgtacagatacttcgcctacgactgtgagagctcgttcccaggcatttcaactggacccatgaagaaagtggatgacatcatctgtgactctcgatattctgacattgagccctcgacagaaggagaggtaatatttcgtgctttagatcctgctttcaaaattgaagacccttatagtccaaggatacagaatctattaaaaatcaccaacttgagaatcaagtttgtgaaactgcacaccttgggggataaccttttggactccagaatggaaatccgagagaagtactattacgctgtttatgatatggtggttcgaggg

SEQ ID NO: 96 - protein, 248 aa, Artificial Sequence
  Mouse Laminin  1 LN
QEPEFSYGCAEGSCYPATGDLLIGRAQKLSVTSTCGLHKPEPYCIVSHLQEDKKCFICDSRDPYHETLNPDSHLIENVVTTFAPNRLKIWWQSENGVENVTIQLDLEAEFHFTHLIMTFKTFRPAAMLIERSSDFGKTWGVYRYFAYDCESSFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRG

SEQ ID NO: 97 - DNA, 744 bp, Artificial Sequence
  Human Laminin  1 LN domain [DNA, 744 bp]
caggaacccgagttcagctacggctgcgcagaaggcagctgctatcccgccacgggcgaccttctcatcggccgagcacagaagctttcggtgacctcgacgtgcgggctgcacaagcccgaaccctactgtatcgtcagccacttgcaggaggacaaaaaatgcttcatatgcaattcccaagatccttatcatgagaccctgaatcctgacagccatctcattgaaaatgtggtcactacatttgctccaaaccgccttaagatttggtggcaatctgaaaatggtgtggaaaatgtaactatccaactggatttggaagcagaattccattttactcatctcataatgactttcaagacattccgtccagctgctatgctgatagaacgatcgtccgactttgggaaaacctggggtgtgtatagatacttcgcctatgactgtgaggcctcgtttccaggcatttcaactggccccatgaaaaaagtcgatgacataatttgtgattctcgatattctgacattgaaccctcaactgaaggagaggtgatatttcgtgctttagatcctgctttcaaaatagaagatccttatagcccaaggatacagaatttattaaaaattaccaacttgagaatcaagtttgtgaaactgcatactttgggagataaccttctggattccaggatggaaatcagagaaaagtattattatgcagtttatgatatggtggttcgagga

SEQ ID NO: 98 - protein, 248 aa, Artificial Sequence
  Human Laminin  1 LN
QEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRG

SEQ ID NO: 99 - DNA, 192 bp, Artificial Sequence
  Mouse Laminin  1 LEa-1 domain [DNA, 192 bp]
aactgcttctgctatggccacgccagtgaatgcgcccctgtggatggagtcaatgaagaagtggaaggaatggttcacgggcactgcatgtgcagacacaacaccaaaggcctgaactgtgagctgtgcatggatttctaccacgatttgccgtggagacctgctgaaggccggaacagcaacgcctgcaaa

SEQ ID NO: 100 - protein, 64 aa, Artificial Sequence
  Mouse Laminin  1 LEa-1
NCFCYGHASECAPVDGVNEEVEGMVHGHCMCRHNTKGLNCELCMDFYHDLPWRPAEGRNSNACK

SEQ ID NO: 101 - DNA, 192 bp, Artificial Sequence
  Human Laminin  1 LEa-1 [DNA, 192 bp]
aattgcttctgctatggtcatgccagcgaatgtgcccctgtggatggattcaatgaagaagtggaaggaatggttcacggacactgcatgtgcaggcataacaccaagggcttaaactgtgaactctgcatggatttctaccatgatttaccttggagacctgctgaaggccgaaacagcaacgcctgtaaa

SEQ ID NO: 102 - protein, 64 aa, Artificial Sequence
  Human Laminin  1 LEa-1
NCFCYGHASECAPVDGFNEEVEGMVHGHCMCRHNTKGLNCELCMDFYHDLPWRPAEGRNSNACK

SEQ ID NO: 103 - DNA, 189 bp, Artificial Sequence
  Mouse Laminin  1 LEa-2 domain [DNA, 189 bp]
aaatgtaactgcaatgaacattccagctcgtgtcactttgacatggcagtcttcctggctactggcaacgtcagcgggggagtgtgtgataactgtcagcacaacaccatggggcgcaactgtgaacagtgcaaaccgttctacttccagcaccctgagagggacatccgggaccccaatctctgtgaa

SEQ ID NO: 104 - protein, 63 aa, Artificial Sequence
  Mouse Laminin  1 LEa-2
KCNCNEHSSSCHFDMAVFLATGNVSGGVCDNCQHNTMGRNCEQCKPFYFQHPERDIRDPNLCE

SEQ ID NO: 105 - DNA, 189 bp, Artificial Sequence
  Human Laminin  1 LEa-2 [DNA, 189 bp]
aaatgtaactgcaatgaacattccatctcttgtcactttgacatggctgtttacctggccacggggaacgtcagcggaggcgtgtgtgatgactgtcagcacaacaccatggggcgcaactgtgagcagtgcaagccgttttactaccagcacccagagagggacatccgagatcctaatttctgtgaa

SEQ ID NO: 106 - protein, 63 aa, Artificial Sequence
  Human Laminin  1 LEa-
KCNCNEHSISCHFDMAVYLATGNVSGGVCDDCQHNTMGRNCEQCKPFYYQHPERDIRDPNFCE

SEQ ID NO: 107 - DNA, 180 bp, Artificial Sequence
  Mouse Laminin  1 LEa-3 domain [DNA, 180 bp]
ccatgtacctgtgacccagctggttctgagaatggcgggatctgtgatgggtacactgatttttctgtgggtctcattgctggtcagtgtcggtgcaaattgcacgtggagggagagcgctgtgatgtttgtaaagaaggcttctacgacttaagtgctgaagacccgtatggttgtaaa

SEQ ID NO: 108 - protein, 60 aa, Artificial Sequence
  Mouse Laminin  1 LEa-3
PCTCDPAGSENGGICDGYTDFSVGLIAGQCRCKLHVEGERCDVCKEGFYDLSAEDPYGCK

SEQ ID NO: 109 - DNA, 180 bp, Artificial Sequence
  Human Laminin  1 LEa-3 [DNA, 180 bp]
cgatgtacgtgtgacccagctggctctcaaaatgagggaatttgtgacagctatactgatttttctactggtctcattgctggccagtgtcggtgtaaattaaatgtggaaggagaacattgtgatgtttgcaaagaaggcttctatgatttaagcagtgaagatccatttggttgtaaa

SEQ ID NO: 110 - protein, 60 aa, Artificial Sequence
  Human Laminin  1 LEa-3
RCTCDPAGSQNEGICDSYTDFSTGLIAGQCRCKLNVEGEHCDVCKEGFYDLSSEDPFGCK

SEQ ID NO: 111 - DNA, 156 bp, Artificial Sequence
  Mouse Laminin  1 LEa-4 domain [DNA, 156 bp]
tcatgtgcttgcaatcctctgggaacaattcctggtgggaatccttgtgattctgagactggctactgctactgtaagcgcctggtgacaggacagcgctgtgaccagtgcctgccgcagcactggggtttaagcaatgatttggatgggtgtcga

SEQ ID NO: 112 - protein, 52 aa, Artificial Sequence
  Mouse Laminin  1 LEa-4
SCACNPLGTIPGGNPCDSETGYCYCKRLVTGQRCDQCLPQHWGLSNDLDGCR

SEQ ID NO: 113 - DNA, 156 bp, Artificial Sequence
  Human Laminin  1 LEa-4 [DNA, 156 bp]
tcttgtgcttgcaatcctctgggaacaattcctggagggaatccttgtgattccgagacaggtcactgctactgcaagcgtctggtgacaggacagcattgtgaccagtgcctgccagagcactggggcttaagcaatgatttggatggatgtcga

SEQ ID NO: 114 - protein, 52 aa, Artificial Sequence
  Human Laminin  1 LEa-4
SCACNPLGTIPGGNPCDSETGHCYCKRLVTGQHCDQCLPEHWGLSNDLDGCR

SEQ ID NO: 115 - DNA, 99 bp, Artificial Sequence
  Mouse Laminin  1 signal peptide [DNA, 99 bp]
atgacgggcggcgggcgggccgcgctggccctgcagccccgggggcggctgtggccgctgttggctgtgctggcggctgtggcgggctgtgtccgggcg

SEQ ID NO: 116 - protein, 33 aa, Artificial Sequence
  Mouse Laminin  1 signal peptide
MTGGGRAALALQPRGRLWPLLAVLAAVAGCVRA

SEQ ID NO: 117 - DNA, 99 bp, Artificial Sequence
  Human Laminin  1 signal peptide [DNA, 99 bp]
atgagagggagccatcgggccgcgccggccctgcggccccgggggcggctctggcccgtgctggccgtgctggcggcggccgccgcggcgggctgtgcc

SEQ ID NO: 118 - protein, 33 aa, Artificial Sequence
  HUMAN Laminin  1signal peptide
MRGSHRAAPALRPRGRLWPVLAVLAAAAAAGCA

SEQ ID NO: 119 - DNA, 768 bp, Artificial Sequence
  Mouse Laminin  1 LN domain [DNA, 768 bp] (note E/GAG (2) in human  1 vs D/GAC (1)  D or E in mouse  I, but E in crystal structure of mouse LN-LEa)
gccatggactacaaggacgacgatgacaaggagtgcgcggatgagggcgggcggccgcagcgctgcatgccggagtttgttaatgccgccttcaatgtgaccgtggtggctaccaacacgtgtgggactccgcccgaggagtactgcgtgcagactggggtgaccggagtcactaagtcctgtcacctgtgcgacgccggccagcagcacctgcaacacggggcagccttcctgaccgactacaacaaccaggccgacaccacctggtggcaaagccagactatgctggccggggtgcagtaccccaactccatcaacctcacgctgcacctgggaaaggcttttgacatcacttacgtgcgcctcaagttccacaccagccgtccagagagcttcgccatctataagcgcactcgggaagacgggccctggattccttatcagtactacagtgggtcctgtgagaacacgtactcaaaggctaaccgtggcttcatcaggaccggaggggacgagcagcaggccttgtgtactgatgaattcagtgacatttcccccctcaccggtggcaacgtggccttttcaaccctggaaggacggccgagtgcctacaactttgacaacagccctgtgctccaggaatgggtaactgccactgacatcagagtgacgctcaatcgcctgaacacctttggagatgaagtgtttaacgagcccaaagttctcaagtcttactattacgcaatctcagactttgctgtgggcggc

SEQ ID NO: 120 - protein, 249 aa, Artificial Sequence
  Mouse Laminin  1 LN domain
AMDECADEGGRPQRCMPEFVNAAFNVTVVATNTCGTPPEEYCVQTGVTGVTKSCHLCDAGQQHLQHGAAFLTDYNNQADTTWWQSQTMLAGVQYPNSINLTLHLGKAFDITYVRLKFHTSRPESFAIYKRTREDGPWIPYQYYSGSCENTYSKANRGFIRTGGDEQQALCTDEFSDISPLTGGNVAFSTLEGRPSAYNFDNSPVLQEWVTATDIRVTLNRLNTFGDEVFNEPKVLKSYYYAISDFAVGG

SEQ ID NO: 121 - DNA, 753 bp, Artificial Sequence
  Human Laminin  1 LN domain [DNA, 753 bp]
caggcagccatggacgagtgcacggacgagggcgggcggccgcaacgctgcatgcccgagttcgtcaacgccgctttcaacgtgactgtggtggccaccaacacgtgtgggactccgcccgaggaatactgtgtgcagaccggggtgaccggggtcaccaagtcctgtcacctgtgcgacgccgggcagccccacctgcagcacggggcagccttcctgaccgactacaacaaccaggccgacaccacctggtggcaaagccagaccatgctggccggggtgcagtaccccagctccatcaacctcacgctgcacctgggaaaagcttttgacatcacctatgtgcgtctcaagttccacaccagccgcccggagagctttgccatttacaagcgcacatgggaagacgggccctggattccttaccagtactacagtggttcctgcgagaacacctactccaaggcaaaccgcggcttcatcaggacaggaggggacgagcagcaggccttgtgtactgatgaattcagtgacatttctcccctcactgggggcaacgtggccttttctaccctggaaggaaggcccagcgcctataactttgacaatagccctgtgctgcaggaatgggtaactgccactgacatcagtgtaactcttaatcgcctgaacacttttggagatgaagtgtttaacgatcccaaagttctcaagtcctattattatgccatctctgattttgctgtaggtggc

SEQ ID NO: 122 - protein, 251 aa, Artificial Sequence
  Human Laminin  1 LN domain
QAAMDECTDEGGRPQRCMPEFVNAAFNVTVVATNTCGTPPEEYCVQTGVTGVTKSCHLCDAGQPHLQHGAAFLTDYNNQADTTWWQSQTMLAGVQYPSSINLTLHLGKAFDITYVRLKFHTSRPESFAIYKRTWEDGPWIPYQYYSGSCENTYSKANRGFIRTGGDEQQALCTDEFSDISPLTGGNVAFSTLEGRPSAYNFDNSPVLQEWVTATDISVTLNRLNTFGDEVFNDPKVLKSYYYAISDFAVGG

SEQ ID NO: 123 - DNA, 168 bp, Artificial Sequence
  Mouse Laminin  1 LEa 1 domain DNA 68 bp note TGC for cys Durkin et al Biochemistry 27 14
aggtgtaaatgtaacggacatgccagcgagtgtgtaaagaacgagtttgacaaactcatgtgcaactgcaaacataacacatacggagttgactgtgaaaagtgcctgcctttcttcaatgaccggccgtggaggagggcgactgctgagagcgccagcgagtgcctt

SEQ ID NO: 124 - protein, 56 aa, Artificial Sequence
  Mouse Laminin  1 LEa-1
RCKCNGHASECVKNEFDKLMCNCKHNTYGVDCEKCLPFFNDRPWRRATAESASECL

SEQ ID NO: 125 - DNA, 168 bp, Artificial Sequence
  Human Laminin  1 LEa-1 [DNA, 168 bp]
agatgtaaatgtaatggacacgcaagcgagtgtatgaagaacgaatttgataagctggtgtgtaattgcaaacataacacatatggagtagactgtgaaaagtgtcttcctttcttcaatgaccggccgtggaggagggcaactgcggaaagtgccagtgaatgcctg

SEQ ID NO: 126 - protein, 56 aa, Artificial Sequence
  Human Laminin  1 LEa-1
RCKCNGHASECMKNEFDKLVCNCKHNTYGVDCEKCLPFFNDRPWRRATAESASECL

SEQ ID NO: 127 - DNA, 168 bp, Artificial Sequence
  Mouse Laminin  1 LEa-2 domain [DNA, 168 bp]
ccttgtgactgcaatggccgatcccaagagtgctactttgatcctgaactataccgttccactggacatggtggccactgtaccaactgccgggataacacagatggtgccaagtgcgagaggtgccgggagaatttcttccgcctggggaacactgaagcctgctct

SEQ ID NO: 128 - protein, 56 aa, Artificial Sequence
  Mouse Laminin  1 LEa-2
PCDCNGRSQECYFDPELYRSTGHGGHCTNCRDNTDGAKCERCRENFFRLGNTEACS

SEQ ID NO: 129 - DNA, 168 bp, Artificial Sequence
  Human Laminin  1 LEa-2 [DNA, 168 bp]
ccctgtgattgcaatggtcgatcccaggaatgctacttcgaccctgaactctatcgttccactggccatgggggccactgtaccaactgccaggataacacagatggcgcccactgtgagaggtgccgagagaacttcttccgccttggcaacaatgaagcctgctct

SEQ ID NO: 130 - protein, 56 aa, Artificial Sequence
  Human Laminin  1 LEa-2
PCDCNGRSQECYFDPELYRSTGHGGHCTNCQDNTDGAHCERCRENFFRLGNNEACS

SEQ ID NO: 131 - DNA, 141 bp, Artificial Sequence
  Mouse Laminin  1 LEa-3 domain [DNA, 141 bp]
ccgtgccactgcagccctgttggttctctcagcacacagtgtgacagttacggcagatgcagctgtaagccaggagtgatgggtgacaagtgtgaccgttgtcagcctgggttccattccctcactgaggcaggatgcagg

SEQ ID NO: 132 - protein, 47 aa, Artificial Sequence
  Mouse Laminin  1 LEa-3
PCHCSPVGSLSTQCDSYGRCSCKPGVMGDKCDRCQPGFHSLTEAGCR

SEQ ID NO: 133 - DNA, 141 bp, Artificial Sequence
  Human Laminin  1 LEa-3 [DNA, 141 bp]
tcatgccactgtagtcctgtgggctctctaagcacacagtgtgatagttacggcagatgcagctgtaagccaggagtgatgggggacaaatgtgaccgttgccagcctggattccattctctcactgaagcaggatgcagg

SEQ ID NO: 134 - protein, 47 aa, Artificial Sequence
  Human Laminin  1 LEa-3
SCHCSPVGSLSTQCDSYGRCSCKPGVMGDKCDRCQPGFHSLTEAGCR

SEQ ID NO: 135 - DNA, 150 bp, Artificial Sequence
  Mouse Laminin  1 LEa-4 [DNA, 150 bp]
ccatgctcctgcgatcttcggggcagcacagacgagtgtaatgttgaaacaggaagatgcgtttgcaaagacaatgttgaaggcttcaactgtgagagatgcaaacctggattttttaatctggagtcatctaatcctaagggctgcaca

SEQ ID NO: 136 - protein, 50 aa, Artificial Sequence
  Mouse Laminin  1 LEa-4
PCSCDLRGSTDECNVETGRCVCKDNVEGFNCERCKPGFFNLESSNPKGCT

SEQ ID NO: 137 - DNA, 150 bp, Artificial Sequence
  Human Laminin  1 LEa-4 [DNA, 150 bp]
ccatgctcttgtgatccctctggcagcatagatgaatgtaatgttgaaacaggaagatgtgtttgcaaagacaatgtcgaaggcttcaattgtgaaagatgcaaacctggattttttaatctggaatcatctaatcctcggggttgcaca

SEQ ID NO: 138 - protein, 50 aa, Artificial Sequence
  Human Laminin  1 LEa-4
PCSCDPSGSIDECNVETGRCVCKDNVEGFNCERCKPGFFNLESSNPRGCT

SEQ ID NO: 139 - DNA, 531 bp, Artificial Sequence
  Mouse agrin LG1 domain [DNA, 531 bp]
ccctctgtgccagcttttaagggccactccttcttggccttccccaccctccgagcctaccacacgctgcgtctggcactagaattccgggcgctggagacagagggactgctgctctacaatggcaatgcacgtggcaaagatttcctggctctggctctgttggatggtcatgtacagttcaggttcgacacgggctcagggccggcggtgctaacaagcttagtgccagtggaaccgggacggtggcaccgcctcgagttgtcacggcattggcggcagggcacactttctgtggatggcgaggctcctgttgtaggtgaaagtccgagtggcactgatggcctcaacttggacacgaagctctatgtgggtggtctcccagaagaacaagttgccacggtgcttgatcggacctctgtgggcatcggcctgaaaggatgcattcgtatgttggacatcaacaaccagcagctggagctgagcgattggcagagggctgtggttcaaagctctggtgtgggggaatgc

SEQ ID NO: 140 - protein, 177 aa, Artificial Sequence
  Mouse agrin LG1 domain
PSVPAFKGHSFLAFPTLRAYHTLRLALEFRALETEGLLLYNGNARGKDFLALALLDGHVQFRFDTGSGPAVLTSLVPVEPGRWHRLELSRHWRQGTLSVDGEAPVVGESPSGTDGLNLDTKLYVGGLPEEQVATVLDRTSVGIGLKGCIRMLDINNQQLELSDWQRAVVQSSGVGEC

SEQ ID NO: 141 - DNA, 531 bp, Artificial Sequence
  Human Agrin LG1 [DNA, 531 bp]
gcccctgtgccggccttcgagggccgctccttcctggccttccccactctccgcgcctaccacacgctgcgcctggcactggaattccgggcgctggagcctcaggggctgctgctgtacaatggcaacgcccggggcaaggacttcctggcattggcgctgctagatggccgcgtgcagctcaggtttgacacaggttcggggccggcggtgctgaccagtgccgtgccggtagagccgggccagtggcaccgcctggagctgtcccggcactggcgccggggcaccctctcggtggatggtgagacccctgttctgggcgagagtcccagtggcaccgacggcctcaacctggacacagacctctttgtgggcggcgtacccgaggaccaggctgccgtggcgctggagcggaccttcgtgggcgccggcctgagggggtgcatccgtttgctggacgtcaacaaccagcgcctggagcttggcattgggccgggggctgccacccgaggctctggcgtgggcgagtgc

SEQ ID NO: 142 - protein, 177 aa, Artificial Sequence
  Human Agrin LG1
APVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGEC

SEQ ID NO: 143 - DNA, 114 bp, Artificial Sequence
  Mouse agrin EGF-like domain 2 [DNA, 114 bp]
ggagaccatccctgctcacctaacccctgccatggcggggccctctgccaggccctggaggctggcgtgttcctctgtcagtgcccacctggccgctttggcccaacttgtgca

SEQ ID NO: 144 - protein, 38 aa, Artificial Sequence
  Mouse agrin EGF-like domain 2
GDHPCSPNPCHGGALCQALEAGVFLCQCPPGRFGPTCA

SEQ ID NO: 145 - DNA, 114 bp, Artificial Sequence
  Human agrin EGF-like domain 2 [DNA, 114 bp]
ggggaccacccctgcctgcccaacccctgccatggcggggccccatgccagaacctggaggctggaaggttccattgccagtgcccgcccggccgcgtcggaccaacctgtgcc

SEQ ID NO: 146 - protein, 38 aa, Artificial Sequence
  Human Agrin EGF-like 2
GDHPCLPNPCHGGAPCQNLEAGRFHCQCPPGRVGPTCA

SEQ ID NO: 147 - DNA, 117 bp, Artificial Sequence
  Mouse agrin EGF-like domain 3 [DNA, 117 bp]
gatgaaaagaacccctgccaaccgaacccctgccacgggtcagccccctgccatgtgctttccaggggtggggccaagtgtgcgtgccccctgggacgcagtggttccttctgtgag

SEQ ID NO: 148 - protein, 39 aa, Artificial Sequence
  Mouse agrin EGF-like domain 3
DEKNPCQPNPCHGSAPCHVLSRGGAKCACPLGRSGSFCE

SEQ ID NO: 149 - DNA, 117 bp, Artificial Sequence
  Human Agrin EGF-like 3 [DNA, 117 bp]
gatgagaagagcccctgccagcccaacccctgccatggggcggcgccctgccgtgtgctgcccgagggtggtgctcagtgcgagtgccccctggggcgtgagggcaccttctgccag

SEQ ID NO: 150 - protein, 39 aa, Artificial Sequence
  Human Agrin EGF-like 3
DEKSPCQPNPCHGAAPCRVLPEGGAQCECPLGREGTFCQ

SEQ ID NO: 151 - DNA, 27 bp, Artificial Sequence
  Mouse agrin LG Spacer-1 [DNA, 27 bp]
acagtcctggagaatgctggctcccgg

SEQ ID NO: 152 - protein, 9 aa, Artificial Sequence
  Mouse agrin spacer domain-1
TVLENAGSR

SEQ ID NO: 153 - DNA, 27 bp, Artificial Sequence
  Human spacer [DNA, 27 bp]
acagcctcggggcaggacggctctggg

SEQ ID NO: 154 - protein, 9 aa, Artificial Sequence
  Human spacer
TASGQDGSG

SEQ ID NO: 155 - DNA, 537 bp, Artificial Sequence
  Mouse agrin LG2 domain [DNA, 537 bp]
cccttcctggctgactttaatggcttctcctacctggaactgaaaggcttgcacaccttcgagagagacctaggggagaagatggcgctggagatggtgttcttggctcgtgggcccagtggcttactcctctacaatgggcagaagacggatggcaagggggactttgtatccctggccctgcataaccggcacctagagttccgctatgaccttggcaagggggctgcaatcatcaggagcaaagagcccatagccctgggcacctgggttagggtattcctggaacgaaatggccgcaagggtgcccttcaagtgggtgatgggccccgtgtgctaggggaatctccggtcccgcacaccatgctcaacctcaaggagcccctctatgtggggggagctcctgacttcagcaagctggctcggggcgctgcagtggcctccggctttgatggtgccatccagctggtgtctctaagaggccatcagctgctgactcaggagcatgtgttgcgggcagtagatgtagcgcctttt

SEQ ID NO: 156 - protein, 179 aa, Artificial Sequence
  Mouse agrin LG2 domain
PFLADFNGFSYLELKGLHTFERDLGEKMALEMVFLARGPSGLLLYNGQKTDGKGDFVSLALHNRHLEFRYDLGKGAAIIRSKEPIALGTWVRVFLERNGRKGALQVGDGPRVLGESPVPHTMLNLKEPLYVGGAPDFSKLARGAAVASGFDGAIQLVSLRGHQLLTQEHVLRAVDVAPF

SEQ ID NO: 157 - DNA, 537 bp, Artificial Sequence
  Human Agrin G2 [DNA, 537 bp]
cccttcctggctgacttcaacggcttctcccacctggagctgagaggcctgcacacctttgcacgggacctgggggagaagatggcgctggaggtcgtgttcctggcacgaggccccagcggcctcctgctctacaacgggcagaagacggacggcaagggggacttcgtgtcgctggcactgcgggaccgccgcctggagttccgctacgacctgggcaagggggcagcggtcatcaggagcagggagccagtcaccctgggagcctggaccagggtctcactggagcgaaacggccgcaagggtgccctgcgtgtgggcgacggcccccgtgtgttgggggagtccccggttccgcacaccgtcctcaacctgaaggagccgctctacgtagggggcgctcccgacttcagcaagctggcccgtgctgctgccgtgtcctctggcttcgacggtgccatccagctggtctccctcggaggccgccagctgctgaccccggagcacgtgctgcggcaggtggacgtcacgtccttt

SEQ ID NO: 158 - protein, 179 aa, Artificial Sequence
  Human Agrin LG2
PFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSF

SEQ ID NO: 159 - DNA, 120 bp, Artificial Sequence
  Mouse agrin EGF-like domain 4 [DNA, 120 bp]
gcaggccacccttgtacccaggccgtggacaacccctgccttaatgggggctcctgtatcccgagggaagccacttatgagtgcctgtgtcctgggggcttctctgggctgcactgcgag

SEQ ID NO: 160 - protein, 40 aa, Artificial Sequence
  Mouse agrin EGF-like domain 4
AGHPCTQAVDNPCLNGGSCIPREATYECLCPGGFSGLHCE

SEQ ID NO: 161 - DNA, 120 bp, Artificial Sequence
  Human Agrin Egf-like 4 [DNA, 120 bp]
gcaggtcacccctgcacccgggcctcaggccacccctgcctcaatggggcctcctgcgtcccgagggaggctgcctatgtgtgcctgtgtcccgggggattctcaggaccgcactgcgag

SEQ ID NO: 162 - protein, 40 aa, Artificial Sequence
  Human Agrin EGF-like 4
AGHPCTRASGHPCLNGASCVPREAAYVCLCPGGFSGPHCE

SEQ ID NO: 163 - DNA, 30 bp, Artificial Sequence
  Mouse agrin LG Spacer-2 [DNA, 30 bp]
aaggggatagttgagaagtcagtgggggac

SEQ ID NO: 164 - protein, 10 aa, Artificial Sequence
  Mouse agrin LG Spacer-2
KGIVEKSVGD

SEQ ID NO: 165 - DNA, 30 bp, Artificial Sequence
  Human Spacer [30 bp]
aaggggctggtggagaagtcagcgggggac

SEQ ID NO: 166 - protein, 10 aa, Artificial Sequence
  Human Spacer
KGLVEKSAGD

SEQ ID NO: 167 - DNA, 537 bp, Artificial Sequence
  Mouse agrin LG3 domain [DNA, 537 bp]
ctagaaacactggcctttgatgggcggacctacatcgagtacctcaatgctgtgactgagagtgagaaagcgctgcagagcaaccactttgagctgagcttacgcactgaggccacgcaggggctggtgctgtggattggaaaggttggagaacgtgcagactacatggctctggccattgtggatgggcacctacaactgagctatgacctaggctcccagccagttgtgctgcgctccactgtgaaggtcaacaccaaccgctggcttcgagtcagggctcacagggagcacagggaaggttcccttcaggtgggcaatgaagcccctgtgactggctcttccccgctgggtgccacacaattggacacagatggagccctgtggcttggaggcctacagaagcttcctgtggggcaggctctccccaaggcctatggcacgggttttgtgggctgtctgcgggacgtggtagtgggccatcgccagctgcatctgctggaggacgctgtcaccaaaccagagctaagaccctgc

SEQ ID NO: 168 - protein, 179 aa, Artificial Sequence
  Mouse agrin LG3 domain
LETLAFDGRTYIEYLNAVTESEKALQSNHFELSLRTEATQGLVLWIGKVGERADYMALAIVDGHLQLSYDLGSQPVVLRSTVKVNTNRWLRVRAHREHREGSLQVGNEAPVTGSSPLGATQLDTDGALWLGGLQKLPVGQALPKAYGTGFVGCLRDVVVGHRQLHLLEDAVTKPELRPC

SEQ ID NO: 169 - DNA, 537 bp, Artificial Sequence
  Human Agrin LG3 [DNA, 537 bp]
gtggataccttggcctttgacgggcggacctttgtcgagtacctcaacgctgtgaccgagagcgagaaggcactgcagagcaaccactttgaactgagcctgcgcactgaggccacgcaggggctggtgctctggagtggcaaggccacggagcgggcagactatgtggcactggccattgtggacgggcacctgcaactgagctacaacctgggctcccagcccgtggtgctgcgttccaccgtgcccgtcaacaccaaccgctggttgcgggtcgtggcacatagggagcagagggaaggttccctgcaggtgggcaatgaggcccctgtgaccggctcctccccgctgggcgccacgcagctggacactgatggagccctgtggcttgggggcctgccggagctgcccgtgggcccagcactgcccaaggcctacggcacaggctttgtgggctgcttgcgggacgtggtggtgggccggcacccgctgcacctgctggaggacgccgtcaccaagccagagctgcggccctgc

SEQ ID NO: 170 - protein, 179 aa, Artificial Sequence
  Human Agrin LG3
VDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPC

SEQ ID NO: 171 - DNA, 3501 bp, Artificial Sequence
  Murine variant alpha LNNd2 ABCDHIJNOP with endogenous signal peptide and no tags
atgaggggcagcggcaccggcgccgccctgctggtgctgctggccagcgtgctgtgggtgaccgtgaggagccagcagagaggcttgttccctgccattctcaacctggccaccaatgcccacatcagcgccaatgctacctgtggagagaaggggcctgagatgttctgcaaactcgtggagcacgtgccgggccggcctgttcgacacgcccaatgccgggtctgtgacggtaacagtacgaatcctagagagcgccatccgatatcacacgcaatcgatggcaccaacaactggtggcagagccccagtattcagaatgggagagagtatcactgggtcactgtcaccctggacttacggcaggtctttcaagttgcatacatcatcattaaagctgccaatgcccctcggcctggaaactggattttggagcgctccgtggatggcgtcaagttcaaaccctggcagtactatgccgtcagcgatacagagtgtttgacccgctacaaaataactccacggcggggacctcccacttacagagcagacaacgaagtcatctgcacctcgtattattcaaagctggtgccacttgaacatggagagattcacacatcactcatcaatggcagacccagcgctgacgacccctcaccccagttgctggaattcacctcagcacggtacattcgccttcgtcttcagcgcatcagaacactcaacgcagacctcatgacccttagccatcgggacctcagagaccttgaccccattgtcacaagacgttattactattcgataaaagacatttccgttggaggcatgtgcatttgctacggccatgccagcagctgcccgtgggatgaagaagcaaagcaactacagtgtcagtgtgaacacaatacgtgtggcgagagctgcgacaggtgctgtcctggctaccatcagcagccctggaggcccggaaccatttcctccggcaacgagtgtgaggaatgcaactgtcacaacaaagccaaagattgttactatgacagcagtgttgcaaaggagaggagaagcctgaacactgccgggcagtacagtggaggaggggtttgtgtcaactgctcgcagaataccacagggatcaactgtgaaacctgtatcgaccagtattacagacctcacaaggtatctccttatgatgaccacccttgccgtcagcagacttgtgccaacaatagacaccagtgctccgtgcatgcagagtgcagagactatgctactggcttctgctgcaggtgtgtggccaactacacaggcaatggcagacagtgcgtggcagaaggctctccacaacgggtcaatggcaaggtgaagggaaggatcttcgtggggagcagccaggtccccgtggtgtttgagaacactgacctgcactcctatgtggtgatgaaccacgggcgctcttacacagccatcagcaccatccctgaaaccgtcggctactctctgctccccctggcacccattggaggcatcatcggatggatgtttgcagtggagcaggatgggttcaagaatgggtttagcatcactgggggcgagtttacccggcaagctgaggtgaccttcctggggcacccaggcaagctggtcctgaagcagcagttcagcggtattgatgaacatggacacctgaccatcagcacggagctggagggccgcgtgccgcagatcccctatggagcctcggtgcacattgagccctacaccgaactgtaccactactccagctcagtgatcacttcctcctccacccgggagtacacggtgatggagcctgatcaggacggcgctgcaccctcacacacccatatttaccagtggcgtcagaccatcaccttccaggagtgtgcccacgatgacgccaggccagccctgcccagcacccagcagctctctgtggacagcgtgtttgtcctgtacaacaaggaggagaggatcttgcgctatgccctcagcaactccatcgggcctgtgagggatggctcccctgatgcccttcagaatccatgctacattggcacccatgggtgtgacagcaatgctgcctgtcgccctggccctggaacacagttcacctgcgaatgctccatcggcttccgaggagacgggcagacttgctatgatattgatgagtgttcagagcagccttcccgctgtgggaaccatgcggtctgcaacaacctcccaggaaccttccgctgcgagtgtgtagagggctaccacttctcagacaggggaacatgcgtgtgtcaactggaacgagagcacatccttggagcagccggcggggcagatgcacagcggcccaccctgcaggggatgtttgtgcctcagtgtgatgaatatggacactatgtacccacccagtgtcaccacagcactggctactgctggtgtgtggaccgagatggtcgggagctggagggtagccgtaccccacctgggatgaggcccccgtgtctgagtacagtggctcctcctattcaccagggaccagtagtacctacagctgtcatccccctgcctccagggacacacttactctttgctcagactggaaagattgaacgcctgcccctggaaagaaacaccatgaagaagacagaacgcaaggcctttctccatatccctgcaaaagtcatcattggactggcctttgactgcgtggacaaggtggtttactggacagacatcagcgagccttccattgggagagccagcctccacggtggagagccaaccaccatcattcgacaagatcttggaagccctgaaggcattgcccttgaccatcttggtcgaaccatcttctggacggactctcagttggatcgaatagaagttgcaaagatggatggcacccagcgccgagtgctgtttgacacgggtttggtgaatcccagaggcattgtgacagaccccgtaagagggaacctttattggacagattggaacagagataatcccaaaattgagacttctcacatggatggcaccaaccggaggattctcgcacaggacaacctgggcttgcccaatggtctgacctttgatgcattctcatctcagctttgctgggtggatgcaggcacccatagggcagaatgcctgaacccagctcagcctggcagacgcaaagttctcgaagggctccagtatcctttcgctgtgactagctatgggaagaatttgtactacacagactggaagacgaattcagtgattgccatggaccttgctatatccaaagagatggataccttccacccacacaagcagacccggctatatggcatcaccatcgccctgtcccagtgtccccaaggccacaattactgctcagtgaataatggtggatgtacccacctctgcttgcccactccagggagcaggacctgccgatgtcctgacaacaccctgggagttgactgcattgaacggaaatga

SEQ ID NO: 172 - protein, 1166 aa, Artificial Sequence
  Murine variant alpha LNNd2 ABCDHIJNOP with endogenous signal peptide and no tags
MRGSGTGAALLVLLASVLWVTVRSQQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGGMCICYGHASSCPWDEEAKQLQCQCEHNTCGESCDRCCPGYHQQPWRPGTISSGNECEECNCHNKAKDCYYDSSVAKERRSLNTAGQYSGGGVCVNCSQNTTGINCETCIDQYYRPHKVSPYDDHPCRQQTCANNRHQCSVHAECRDYATGFCCRCVANYTGNGRQCVAEGSPQRVNGKVKGRIFVGSSQVPVVFENTDLHSYVVMNHGRSYTAISTIPETVGYSLLPLAPIGGIIGWMFAVEQDGFKNGFSITGGEFTRQAEVTFLGHPGKLVLKQQFSGIDEHGHLTISTELEGRVPQIPYGASVHIEPYTELYHYSSSVITSSSTREYTVMEPDQDGAAPSHTHIYQWRQTITFQECAHDDARPALPSTQQLSVDSVFVLYNKEERILRYALSNSIGPVRDGSPDALQNPCYIGTHGCDSNAACRPGPGTQFTCECSIGFRGDGQTCYDIDECSEQPSRCGNHAVCNNLPGTFRCECVEGYHFSDRGTCVCQLEREHILGAAGGADAQRPTLQGMFVPQCDEYGHYVPTQCHHSTGYCWCVDRDGRELEGSRTPPGMRPPCLSTVAPPIHQGPVVPTAVIPLPPGTHLLFAQTGKIERLPLERNTMKKTERKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPHKQTRLYGITIALSQCPQGHNYCSVNNGGCTHLCLPTPGSRTCRCPDNTLGVDCIERK

SEQ ID NO: 173 - DNA, 3291 bp, Artificial Sequence
  Murine variant alpha LNNd with ABCHIJNOP endogenous signal peptide and no tags
atgaggggcagcggcaccggcgccgccctgctggtgctgctggccagcgtgctgtgggtgaccgtgaggagccagcagagaggcttgttccctgccattctcaacctggccaccaatgcccacatcagcgccaatgctacctgtggagagaaggggcctgagatgttctgcaaactcgtggagcacgtgccgggccggcctgttcgacacgcccaatgccgggtctgtgacggtaacagtacgaatcctagagagcgccatccgatatcacacgcaatcgatggcaccaacaactggtggcagagccccagtattcagaatgggagagagtatcactgggtcactgtcaccctggacttacggcaggtctttcaagttgcatacatcatcattaaagctgccaatgcccctcggcctggaaactggattttggagcgctccgtggatggcgtcaagttcaaaccctggcagtactatgccgtcagcgatacagagtgtttgacccgctacaaaataactccacggcggggacctcccacttacagagcagacaacgaagtcatctgcacctcgtattattcaaagctggtgccacttgaacatggagagattcacacatcactcatcaatggcagacccagcgctgacgacccctcaccccagttgctggaattcacctcagcacggtacattcgccttcgtcttcagcgcatcagaacactcaacgcagacctcatgacccttagccatcgggacctcagagaccttgaccccattgtcacaagacgttattactattcgataaaagacatttccgttggaggcatgtgcatttgctacggccatgccagcagctgcccgtgggatgaagaagcaaagcaactacagtgtcagtgtgaacacaatacgtgtggcgagagctgcgacaggtgctgtcctggctaccatcagcagccctggaggcccggaaccatttcctccggcaacgagtgtgagcagcagacttgtgccaacaatagacaccagtgctccgtgcatgcagagtgcagagactatgctactggcttctgctgcaggtgtgtggccaactacacaggcaatggcagacagtgcgtggcagaaggctctccacaacgggtcaatggcaaggtgaagggaaggatcttcgtggggagcagccaggtccccgtggtgtttgagaacactgacctgcactcctatgtggtgatgaaccacgggcgctcttacacagccatcagcaccatccctgaaaccgtcggctactctctgctccccctggcacccattggaggcatcatcggatggatgtttgcagtggagcaggatgggttcaagaatgggtttagcatcactgggggcgagtttacccggcaagctgaggtgaccttcctggggcacccaggcaagctggtcctgaagcagcagttcagcggtattgatgaacatggacacctgaccatcagcacggagctggagggccgcgtgccgcagatcccctatggagcctcggtgcacattgagccctacaccgaactgtaccactactccagctcagtgatcacttcctcctccacccgggagtacacggtgatggagcctgatcaggacggcgctgcaccctcacacacccatatttaccagtggcgtcagaccatcaccttccaggagtgtgcccacgatgacgccaggccagccctgcccagcacccagcagctctctgtggacagcgtgtttgtcctgtacaacaaggaggagaggatcttgcgctatgccctcagcaactccatcgggcctgtgagggatggctcccctgatgcccttcagaatccatgctacattggcacccatgggtgtgacagcaatgctgcctgtcgccctggccctggaacacagttcacctgcgaatgctccatcggcttccgaggagacgggcagacttgctatgatattgatgagtgttcagagcagccttcccgctgtgggaaccatgcggtctgcaacaacctcccaggaaccttccgctgcgagtgtgtagagggctaccacttctcagacaggggaacatgcgtgtgtcaactggaacgagagcacatccttggagcagccggcggggcagatgcacagcggcccaccctgcaggggatgtttgtgcctcagtgtgatgaatatggacactatgtacccacccagtgtcaccacagcactggctactgctggtgtgtggaccgagatggtcgggagctggagggtagccgtaccccacctgggatgaggcccccgtgtctgagtacagtggctcctcctattcaccagggaccagtagtacctacagctgtcatccccctgcctccagggacacacttactctttgctcagactggaaagattgaacgcctgcccctggaaagaaacaccatgaagaagacagaacgcaaggcctttctccatatccctgcaaaagtcatcattggactggcctttgactgcgtggacaaggtggtttactggacagacatcagcgagccttccattgggagagccagcctccacggtggagagccaaccaccatcattcgacaagatcttggaagccctgaaggcattgcccttgaccatcttggtcgaaccatcttctggacggactctcagttggatcgaatagaagttgcaaagatggatggcacccagcgccgagtgctgtttgacacgggtttggtgaatcccagaggcattgtgacagaccccgtaagagggaacctttattggacagattggaacagagataatcccaaaattgagacttctcacatggatggcaccaaccggaggattctcgcacaggacaacctgggcttgcccaatggtctgacctttgatgcattctcatctcagctttgctgggtggatgcaggcacccatagggcagaatgcctgaacccagctcagcctggcagacgcaaagttctcgaagggctccagtatcctttcgctgtgactagctatgggaagaatttgtactacacagactggaagacgaattcagtgattgccatggaccttgctatatccaaagagatggataccttccacccacacaagcagacccggctatatggcatcaccatcgccctgtcccagtgtccccaaggccacaattactgctcagtgaataatggtggatgtacccacctctgcttgcccactccagggagcaggacctgccgatgtcctgacaacaccctgggagttgactgcattgaacggaaatga

SEQ ID NO: 174 - protein, 1096 aa, Artificial Sequence
  Murine variant alpha LNNd with ABCHIJNOP endogenous signal peptide and no tags
MRGSGTGAALLVLLASVLWVTVRSQQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGGMCICYGHASSCPWDEEAKQLQCQCEHNTCGESCDRCCPGYHQQPWRPGTISSGNECEQQTCANNRHQCSVHAECRDYATGFCCRCVANYTGNGRQCVAEGSPQRVNGKVKGRIFVGSSQVPVVFENTDLHSYVVMNHGRSYTAISTIPETVGYSLLPLAPIGGIIGWMFAVEQDGFKNGFSITGGEFTRQAEVTFLGHPGKLVLKQQFSGIDEHGHLTISTELEGRVPQIPYGASVHIEPYTELYHYSSSVITSSSTREYTVMEPDQDGAAPSHTHIYQWRQTITFQECAHDDARPALPSTQQLSVDSVFVLYNKEERILRYALSNSIGPVRDGSPDALQNPCYIGTHGCDSNAACRPGPGTQFTCECSIGFRGDGQTCYDIDECSEQPSRCGNHAVCNNLPGTFRCECVEGYHFSDRGTCVCQLEREHILGAAGGADAQRPTLQGMFVPQCDEYGHYVPTQCHHSTGYCWCVDRDGRELEGSRTPPGMRPPCLSTVAPPIHQGPVVPTAVIPLPPGTHLLFAQTGKIERLPLERNTMKKTERKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPHKQTRLYGITIALSQCPQGHNYCSVNNGGCTHLCLPTPGSRTCRCPDNTLGVDCIERK

SEQ ID NO: 175 - DNA, 3165 bp, Artificial sequence
  Murine variant alpha LNNd ABCHINOP with endogenous signal peptide and no tags
atgaggggcagcggcaccggcgccgccctgctggtgctgctggccagcgtgctgtgggtgaccgtgaggagccagcagagaggcttgttccctgccattctcaacctggccaccaatgcccacatcagcgccaatgctacctgtggagagaaggggcctgagatgttctgcaaactcgtggagcacgtgccgggccggcctgttcgacacgcccaatgccgggtctgtgacggtaacagtacgaatcctagagagcgccatccgatatcacacgcaatcgatggcaccaacaactggtggcagagccccagtattcagaatgggagagagtatcactgggtcactgtcaccctggacttacggcaggtctttcaagttgcatacatcatcattaaagctgccaatgcccctcggcctggaaactggattttggagcgctccgtggatggcgtcaagttcaaaccctggcagtactatgccgtcagcgatacagagtgtttgacccgctacaaaataactccacggcggggacctcccacttacagagcagacaacgaagtcatctgcacctcgtattattcaaagctggtgccacttgaacatggagagattcacacatcactcatcaatggcagacccagcgctgacgacccctcaccccagttgctggaattcacctcagcacggtacattcgccttcgtcttcagcgcatcagaacactcaacgcagacctcatgacccttagccatcgggacctcagagaccttgaccccattgtcacaagacgttattactattcgataaaagacatttccgttggaggcatgtgcatttgctacggccatgccagcagctgcccgtgggatgaagaagcaaagcaactacagtgtcagtgtgaacacaatacgtgtggcgagagctgcgacaggtgctgtcctggctaccatcagcagccctggaggcccggaaccatttcctccggcaacgagtgtgagcagcagacttgtgccaacaatagacaccagtgctccgtgcatgcagagtgcagagactatgctactggcttctgctgcaggtgtgtggccaactacacaggcaatggcagacagtgcgtggcagaaggctctccacaacgggtcaatggcaaggtgaagggaaggatcttcgtggggagcagccaggtccccgtggtgtttgagaacactgacctgcactcctatgtggtgatgaaccacgggcgctcttacacagccatcagcaccatccctgaaaccgtcggctactctctgctccccctggcacccattggaggcatcatcggatggatgtttgcagtggagcaggatgggttcaagaatgggtttagcatcactgggggcgagtttacccggcaagctgaggtgaccttcctggggcacccaggcaagctggtcctgaagcagcagttcagcggtattgatgaacatggacacctgaccatcagcacggagctggagggccgcgtgccgcagatcccctatggagcctcggtgcacattgagccctacaccgaactgtaccactactccagctcagtgatcacttcctcctccacccgggagtacacggtgatggagcctgatcaggacggcgctgcaccctcacacacccatatttaccagtggcgtcagaccatcaccttccaggagtgtgcccacgatgacgccaggccagccctgcccagcacccagcagctctctgtggacagcgtgtttgtcctgtacaacaaggaggagaggatcttgcgctatgccctcagcaactccatcgggcctgtgagggatggctcccctgatgcccttcagaatccatgctacattggcacccatgggtgtgacagcaatgctgcctgtcgccctggccctggaacacagttcacctgcgaatgctccatcggcttccgaggagacgggcagacttgctattgtcaactggaacgagagcacatccttggagcagccggcggggcagatgcacagcggcccaccctgcaggggatgtttgtgcctcagtgtgatgaatatggacactatgtacccacccagtgtcaccacagcactggctactgctggtgtgtggaccgagatggtcgggagctggagggtagccgtaccccacctgggatgaggcccccgtgtctgagtacagtggctcctcctattcaccagggaccagtagtacctacagctgtcatccccctgcctccagggacacacttactctttgctcagactggaaagattgaacgcctgcccctggaaagaaacaccatgaagaagacagaacgcaaggcctttctccatatccctgcaaaagtcatcattggactggcctttgactgcgtggacaaggtggtttactggacagacatcagcgagccttccattgggagagccagcctccacggtggagagccaaccaccatcattcgacaagatcttggaagccctgaaggcattgcccttgaccatcttggtcgaaccatcttctggacggactctcagttggatcgaatagaagttgcaaagatggatggcacccagcgccgagtgctgtttgacacgggtttggtgaatcccagaggcattgtgacagaccccgtaagagggaacctttattggacagattggaacagagataatcccaaaattgagacttctcacatggatggcaccaaccggaggattctcgcacaggacaacctgggcttgcccaatggtctgacctttgatgcattctcatctcagctttgctgggtggatgcaggcacccatagggcagaatgcctgaacccagctcagcctggcagacgcaaagttctcgaagggctccagtatcctttcgctgtgactagctatgggaagaatttgtactacacagactggaagacgaattcagtgattgccatggaccttgctatatccaaagagatggataccttccacccacacaagcagacccggctatatggcatcaccatcgccctgtcccagtgtccccaaggccacaattactgctcagtgaataatggtggatgtacccacctctgcttgcccactccagggagcaggacctgccgatgtcctgacaacaccctgggagttgactgcattgaacggaaatga

SEQ ID NO: 176 - protein, 1054 aa, Artificial Sequence
  Murine alpha LNNd ABCHINOP with endogenous signal peptide and no tags
MRGSGTGAALLVLLASVLWVTVRSQQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGGMCICYGHASSCPWDEEAKQLQCQCEHNTCGESCDRCCPGYHQQPWRPGTISSGNECEQQTCANNRHQCSVHAECRDYATGFCCRCVANYTGNGRQCVAEGSPQRVNGKVKGRIFVGSSQVPVVFENTDLHSYVVMNHGRSYTAISTIPETVGYSLLPLAPIGGIIGWMFAVEQDGFKNGFSITGGEFTRQAEVTFLGHPGKLVLKQQFSGIDEHGHLTISTELEGRVPQIPYGASVHIEPYTELYHYSSSVITSSSTREYTVMEPDQDGAAPSHTHIYQWRQTITFQECAHDDARPALPSTQQLSVDSVFVLYNKEERILRYALSNSIGPVRDGSPDALQNPCYIGTHGCDSNAACRPGPGTQFTCECSIGFRGDGQTCYCQLEREHILGAAGGADAQRPTLQGMFVPQCDEYGHYVPTQCHHSTGYCWCVDRDGRELEGSRTPPGMRPPCLSTVAPPIHQGPVVPTAVIPLPPGTHLLFAQTGKIERLPLERNTMKKTERKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPHKQTRLYGITIALSQCPQGHNYCSVNNGGCTHLCLPTPGSRTCRCPDNTLGVDCIERK

SEQ ID NO: 177 - DNA, 598 bp, Artificial Sequence
  Woodchuck hepatitis posttranscriptional regulatory element (WPRE)
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 178 - DNA, 252 bp, Artificial Sequence
  Truncated woodchuck hepatitis posttranscriptional regulatory element (WPRE)
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttt

SEQ ID NO: 179 - DNA, 3792 bp, Artificial Sequence
  Murine variant alpha LNNd ABCDGHIJKLMNOP with endogenous signal peptide and no tags
atgaggggcagcggcaccggcgccgccctgctggtgctgctggccagcgtgctgtgggtgaccgtgaggagccagcagaggggcctgttccctgccatcctgaacctggccaccaacgcccacatcagcgccaacgccacctgcggcgagaagggccctgagatgttctgcaagctggtggagcacgtgcctggcaggcctgtgaggcacgcccagtgcagggtgtgcgacggcaacagcaccaaccctagggagaggcaccctatcagccacgccatcgacggcaccaacaactggtggcagagccctagcatccagaacggcagggagtaccactgggtgaccgtgaccctggacctgaggcaggtgttccaggtggcctacatcatcatcaaggccgccaacgcccctaggcctggcaactggatcctggagaggagcgtggacggcgtgaagttcaagccttggcagtactacgccgtgagcgacaccgagtgcctgaccaggtacaagatcacccctaggaggggccctcctacctacagggccgacaacgaggtgatctgcaccagctactacagcaagctggtgcctctggagcacggcgagatccacaccagcctgatcaacggcaggcctagcgccgacgaccctagccctcagctgctggagttcaccagcgccaggtacatcaggctgaggctgcagaggatcaggaccctgaacgccgacctgatgaccctgagccacagggacctgagggacctggaccctatcgtgaccaggaggtactactacagcatcaaggacatcagcgtgggcggcatgtgcatctgctacggccacgccagcagctgcccttgggacgaggaggccaagcagctgcagtgccagtgcgagcacaacacctgcggcgagagctgcgacaggtgctgccctggctaccaccagcagccttggaggcctggcaccatcagcagcggcaacgagtgcgaggagtgcaactgccacaacaaggccaaggactgctactacgacagcagcgtggccaaggagaggaggagcctgaacaccgccggccagtacagcggcggcggcgtgtgcgtgaactgcagccagaacaccaccggcatcaactgcgagacctgcatcgaccagtactacaggcctcacaaggtgagcccttacgacgaccacccttgcaggcagcagacctgcgccaacaacaggcaccagtgcagcgtgcacgccgagtgcagggactacgccaccggcttctgctgcaggtgcgtggccaactacaccggcaacggcaggcagtgcgtggccgagggcagccctcagagggtgaacggcaaggtgaagggcaggatcttcgtgggcagcagccaggtgcctgtggtgttcgagaacaccgacctgcacagctacgtggtgatgaaccacggcaggagctacaccgccatcagcaccatccctgagaccgtgggctacagcctgctgcctctggcccctatcggcggcatcatcggctggatgttcgccgtggagcaggacggcttcaagaacggcttcagcatcaccggcggcgagttcaccaggcaggccgaggtgaccttcctgggccaccctggcaagctggtgctgaagcagcagttcagcggcatcgacgagcacggccacctgaccatcagcaccgagctggagggcagggtgcctcagatcccttacggcgccagcgtgcacatcgagccttacaccgagctgtaccactacagcagcagcgtgatcaccagcagcagcaccagggagtacaccgtgatggagcctgaccaggacggcgccgcccctagccacacccacatctaccagtggaggcagaccatcaccttccaggagtgcgcccacgacgacgccaggcctgccctgcctagcacccagcagctgagcgtggacagcgtgttcgtgctgtacaacaaggaggagaggatcctgaggtacgccctgagcaacagcatcggccctgtgagggacggcagccctgacgccctgcagaacccttgctacatcggcacccacggctgcgacagcaacgccgcctgcaggcctggccctggcacccagttcacctgcgagtgcagcatcggcttcaggggcgacggccagacctgctacgacatcgacgagtgcagcgagcagcctagcaggtgcggcaaccacgccgtgtgcaacaacctgcctggcaccttcaggtgcgagtgcgtggagggctaccacttcagcgacaggggcacctgcgtggccgccgaggaccagaggcctatcaactactgcgagaccggcctgcacaactgcgacatccctcagagggcccagtgcatctacatgggcggcagcagctacacctgcagctgcctgcctggcttcagcggcgacggcagggcctgcagggacgtggacgagtgccagcacagcaggtgccaccctgacgccttctgctacaacacccctggcagcttcacctgccagtgcaagcctggctaccagggcgacggcttcaggtgtatgcctggcgaggtgagcaagaccaggtgccagctggagagggagcacatcctgggcgccgccggcggcgccgacgcccagaggcctaccctgcagggcatgttcgtgcctcagtgcgacgagtacggccactacgtgcctacccagtgccaccacagcaccggctactgctggtgcgtggacagggacggcagggagctggagggcagcaggacccctcctggcatgaggcctccttgcctgagcaccgtggcccctcctatccaccagggccctgtggtgcctaccgccgtgatccctctgcctcctggcacccacctgctgttcgcccagaccggcaagatcgagaggctgcctctggagaggaacaccatgaagaagaccgaggccaaggccttcctgcacatccctgccaaggtgatcatcggcctggccttcgactgcgtggacaaggtggtgtactggaccgacatcagcgagcctagcatcggcagggccagcctgcacggcggcgagcctaccaccatcatcaggcaggacctgggcagccctgagggcatcgccctggaccacctgggcaggaccatcttctggaccgacagccagctggacaggatcgaggtggccaagatggacggcacccagaggagggtgctgttcgacaccggcctggtgaaccctaggggcatcgtgaccgaccctgtgaggggcaacctgtactggaccgactggaacagggacaaccctaagatcgagaccagccacatggacggcaccaacaggaggatcctggcccaggacaacctgggcctgcctaacggcctgaccttcgacgccttcagcagccagctgtgctgggtggacgccggcacccacagggccgagtgcctgaaccctgcccagcctggcaggaggaaggtgctggagggcctgcagtaccctttcgccgtgaccagctacggcaagaacctgtactacaccgactggaagaccaacagcgtgatcgccatggacctggccatcagcaaggagatggacaccttccaccctcacaagcagaccaggctgtacggcatcaccatcgccctgagccagtgccctcagggccacaactactgcagcgtgaacaacggcggctgcacccacctgtgcctgcctacccctggcagcaggacctgcaggtgccctgacaacaccctgggcgtggactgcatcgagaggaagtga

SEQ ID NO: 180 - protein, 1263 aa, Artificial Sequence
  Murine variant alpha LNNd ABCDGHIJKLMNOP with endogenous signal peptide and no tags
MRGSGTGAALLVLLASVLWVTVRSQQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGGMCICYGHASSCPWDEEAKQLQCQCEHNTCGESCDRCCPGYHQQPWRPGTISSGNECEECNCHNKAKDCYYDSSVAKERRSLNTAGQYSGGGVCVNCSQNTTGINCETCIDQYYRPHKVSPYDDHPCRQQTCANNRHQCSVHAECRDYATGFCCRCVANYTGNGRQCVAEGSPQRVNGKVKGRIFVGSSQVPVVFENTDLHSYVVMNHGRSYTAISTIPETVGYSLLPLAPIGGIIGWMFAVEQDGFKNGFSITGGEFTRQAEVTFLGHPGKLVLKQQFSGIDEHGHLTISTELEGRVPQIPYGASVHIEPYTELYHYSSSVITSSSTREYTVMEPDQDGAAPSHTHIYQWRQTITFQECAHDDARPALPSTQQLSVDSVFVLYNKEERILRYALSNSIGPVRDGSPDALQNPCYIGTHGCDSNAACRPGPGTQFTCECSIGFRGDGQTCYDIDECSEQPSRCGNHAVCNNLPGTFRCECVEGYHFSDRGTCVAAEDQRPINYCETGLHNCDIPQRAQCIYMGGSSYTCSCLPGFSGDGRACRDVDECQHSRCHPDAFCYNTPGSFTCQCKPGYQGDGFRCMPGEVSKTRCQLEREHILGAAGGADAQRPTLQGMFVPQCDEYGHYVPTQCHHSTGYCWCVDRDGRELEGSRTPPGMRPPCLSTVAPPIHQGPVVPTAVIPLPPGTHLLFAQTGKIERLPLERNTMKKTEAKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPHKQTRLYGITIALSQCPQGHNYCSVNNGGCTHLCLPTPGSRTCRCPDNTLGVDCIERK